(19) United States
(12) Patent Application Publication
(10) Pub. No.: US 2002/0045653 A1
(43) Pub. Date: Apr. 18, 2002

(54) C4 side-chain modified nodulisporic acid analogs

(76) Inventors: Thomas Shih, Jackson, NJ (US); Steven L. Colletti, Princeton Junction, NJ (US); Michael H. Fisher, Ringoes, NJ (US); Peter T. Meinke, Plainfield, NJ (US); Dong Ok, Edison, NJ (US); Prasun K. Chakravarty, Edison, NJ (US); Matthew J. Wyvratt, Mountainside, NJ (US)

Correspondence Address: MERCK AND CO INC, P O BOX 2000, RAHWAY, NJ, 070650907

(21) Appl. No.: 09/901,393

(22) Filed: Jul. 9, 2001

Related U.S. Application Data
(60) Provisional application No. 60/218,202, filed on Jul. 14, 2000.

(51) Int. Cl. C07D 487/06; A61K 31/407

(52) U.S. Cl. 514/410; 548/417

(57) ABSTRACT

The present invention relates to novel nodulosporic acid derivatives, which are acaricidal, antiparasitic, insecticidal and anthelmintic agents.

### BACKGROUND OF THE INVENTION

Nodulosporic acid A and related component nodulisporic acid A1 are antiparasitic agents and ectoparasiticidal agents isolated from the fermentation culture of Nodulisporium sp. MF-5954 (ATCC 74245). These two compounds have the following structures as disclosed in U.S. Pat. No. 5,399,582 and J. G. Ondeyka et al. J. Am. Chem. Soc. 1997, 119(38), 8809-8816.

nodulisporic acid A (compound A)

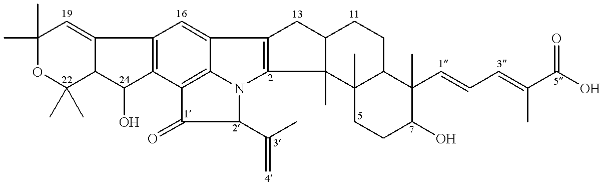

nodulisporic acid A1 (compound B)

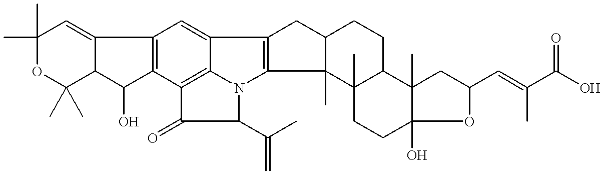

Derivatives of nodulisporic acid are disclosed in U.S. Pat. No. 5,962,499.

### SUMMARY OF THE INVENTION

This invention relates to new acaricidal, antiparasitic, insecticidal and anthelmintic agents related to the nodulisporic acids, to processes for their preparation, compositions thereof, their use in the treatment of parasitic infections, including helminthiasis, in human and animals, and their use in the treatment of parasitic infections in plants or plant products.

### DETAILED DESCRIPTION OF THE INVENTION

The present invention provides compounds having the formula I:

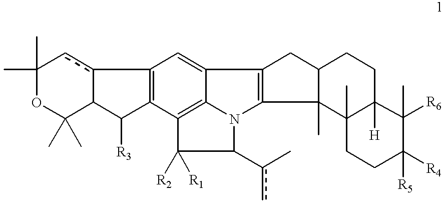

wherein

R₁ is

(1) hydrogen,

(2) optionally substituted C₁-C₁₀ alkyl,

(3) optionally substituted C₂-C₁₀ alkenyl,

(4) optionally substituted C₂-C₁₀ alkynyl,

(5) optionally substituted C₃-C₈ cycloalkyl,

(6) optionally substituted C₅-C₈ cycloalkenyl

where the substitutents on the alkyl, alkenyl, alkynyl, cycloalkyl and cycloalkenyl are 1 to 3 groups independently selected from

(i) C₁-C₅ alkyl,

(ii) X—C₁-C₁₀ alkyl,

(iii) C₃-C₈ cycloalkyl,

(iv) hydroxy,

(v) halogen,

(vi) cyano,

(vii) carboxy,

(viii) NY¹Y²,

(ix) C₁-C₁₀ alkanoylamino, and

(x) aroyl amino wherein said aroyl is optionally substituted with 1 to 3 groups independently selected from R^{f}

(7) aryl C₀-C₅ alkyl wherein said aryl is optionally substituted with 1 to 3 groups independently selected from R^{f},

(8) C¹-C⁵ perfluoroalkyl

(9) a 5- or 6-membered heterocycle optionally substituted by 1 to 3 groups independently selected from hydroxy, oxo, C₁-C₁₀ alkyl and halogen;

R₂, R₃, and R₄ are independently OR^{a}, OCO₂R^{b}, OC(O)NR^{c}R^{d}; or

R₁+R₂ represent ═O, ═NOR^{a}, ═N—NR^{c}R^{d}, ═CCO₂R^{a}, ═CC(O)NR^{c}R^{d}, ═CCN ═CC(O)R^{a}, or ═CR^{a}R^{a};

R₅ is hydrogen, OR^{a} or

R₄+R₅ represent ═O, ═NOR^{a}, ═N—NRCR^{d} or ═CR^{a}R^{a};

R⁶ is

(1) the fragment

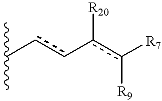

(2) the fragment

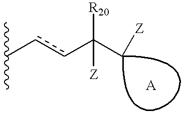

where A is a 5- or 6-membered heterocycle optionally substituted with 1 to 4 groups independently selected from C₁-C₅ alkyl, C₂-C₅ alkenyl, C₁-C₅ perfluoroalkyl, aryl, OR^{a}, NR^{c}R^{d}, oxo, thiono, C(O)R^{a}, C(O)NR^{c}R^{d}, cyano, CO₂R^{b} and halogen, and where a ring nitrogen is present, it is substituted with a group selected from R^{c}; the two Z groups are each OH or together form a bond across the two carbon atoms to which they are attached;

(3) the fragment

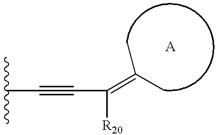

wherein A is as defined above; or

R⁶, R⁴ and the atoms to which they are attached together form the fragment

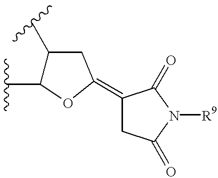

R₇ is

(1) hydrogen,

(2) optionally substituted C₁-C₁₀ alkyl,

(3) optionally substituted C₂-C₁₀ alkenyl,

(4) optionally substituted C₂-C₁₀ alkynyl,

(5) optionally substituted aryl,

(6) optionally substituted C₃-C₈ cycloalkyl,

(7) optionally substituted C₅-C₈ cycloalkenyl,

(8) halogen,

(9) CN,

(10) C(O)R^{a},

(11) CH═NOR^{a},

(12) CO₂R^{b},

(13) C(O)NR^{c}R^{d},

(14) C(O)N(OR^{b})R^{c},

(15) C(O)NR^{c}NR^{c}R^{d},

(16) C(O)NR^{c}SO₂R^{b},

(17) NR^{c}R^{d}

(18) NR^{c}C(O)R^{a},

(19) NR^{c}C(O)OR^{b},

(20) NR^{c}C(O)NR^{c}R^{d},

(21) NR^{c}C(O)SR^{b},

(22) NR^{c}C(O)P(O)(R^{a})₂,

(23) NR^{c}S(O)₂R^{a},

(24) N═C═O,

(25) XR^{a},

(26) OC(O)R^{a},

(27) OSO₂R^{a},

(28) P(O)(OR^{a})2,

(29) 4- to 8-membered heterocycle optionally substituted by 1 to 4 groups independently selected from C₁-C₅ alkyl, C₂-C₅ alkenyl, C₁-C₅ perfluoroalkyl, NR^{c}R^{d}, oxo, thiono, C(O)NR^{c}R^{d}, cyano, aryl, C(O)R^{a}, CO₂R^{b} and halogen,

where the substituents on the optionally substituted alkyl, alkenyl, alkynyl, aryl, cycloalkyl and cycloalkenyl are from 1 to 10 groups independently selected from

(a) halogen,

(b) C₃-C₇ cycloalkyl,

(c) C₁-C₇ alkyl optionally substituted with from 1 to 3 groups independently selected from OR^{a}, oxo, NR^{c}R^{d}, N₃, NR^{c}C(O)R^{a}, NR^{c}SO₂R^{a}, O₂CNR^{c}R^{d}, NR^{c}C(O)NR^{c}R^{d}, CO₂R^{b}, C(O)NR^{c}R^{d}, or a 3- to 8-membered heterocycle optionally substituted with oxo or C(O)R^{a},

(d) C₁-C₇ alkenyl optionally substituted with from 1 to 3 groups independently selected from OR^{a}, oxo, NR^{c}R^{d}, N₃, NR^{c}C(O)R^{a}, NR^{c}SO₂R^{a}, O₂CNR^{c}R^{d}, NR^{c}C(O)R^{c}R^{d}, CO₂R^{b}, C(O)NR^{c}R^{d}, or a 3- to 8-membered heterocycle optionally substituted with oxo or C(O)R^{a},

(e) C₁-C₅ perfluoroalkyl,

(f) aryl optionally substituted with 1 to 3 groups selected from R^{f},

(g) CN,

(h) C(O)R^{a},

(i) CO₂R^{b},

(j) C(O)NR^{c}R^{d},

(k) C(O)N(OR^{b})R^{c},

(l) C(O)NR^{c}NR^{c}R^{d},

(m) C(O)NR^{c}SO₂R^{b},

(n) N═C═O,

(o) N═N═N,

(p) NR^{c}C(O)NR^{c}R^{d},

(q) NR^{c}C(O)P(O)R^{a},

(r) NR^{c}R^{d},

(s) NR^{c}CO₂R^{b},

(t) NR^{c}SO₂R^{a},

(u) NR^{c}C(O)SR^{b},

(v) NR^{c}C(O)R^{a},

(w) ═NOR^{a},

(x) ═NNR^{c}R^{d},

(y) ═NNR^{c}SO₂R^{a}

(z) XR^{a},

(aa) oxo,

(bb) OCO₂R^{b},

(cc) OC(O)NR^{c}R^{d},

(dd) OSO₂R^{a},

(ee) P(O)(OR^{a})₂,

(ff) a 4- to 8-membered heterocycle optionally substituted by 1 to 4 groups independently selected from C₁-C₅ alkyl, C₂-C₅ alkenyl, C₁-C₅ perfluoroalkyl, NR^{c}R^{d}, oxo, thiono, XR^{a}, C(O)NR^{c}R^{d}, cyano, (C₀-C₆-alkyl)aryl, CO₂R^{b} and halogen;

R₉ is a group selected from R₇, with the proviso that R₉ is not methyl when R₇ is CN, C(O)OR^{b}, C(O)N(OR^{b})R^{c}, C(O)NR^{c}R^{d}, NHC(O)OR^{b}, NHC(O)NR^{c}R^{d}, CH₂OR^{a}, CH₂OCO₂R^{b}, CH₂OC(O)NR^{c}R^{d}, C(O)NR^{c}NR^{c}R^{d}, or C(O)NR^{c}SO₂R^{b};

R₂₀ is a group selected from R₇;

___ prepresents a single or a double bond;

R^{a} is

(1) hydrogen,

(2) optionally substituted C₁-C¹⁰ alkyl,

(3) optionally substituted C₃-C₁₀ alkenyl,

(4) optionally substituted C₃-C₁₀ alkynyl,

(5) optionally substituted C₁-C₁₀ alkanoyl,

(6) optionally substituted C₃-C₁₀ alkenoyl,

(7) optionally substituted C₃-C₁₀ alkynoyl,

(8) optionally substituted aroyl,

(9) optionally substituted aryl,

(10) optionally substituted C₃-C₇ cycloalkanoyl,

(11) optionally substituted C₅-C₇ cycloalkenoyl,

(12) optionally substituted C₁-C₁₀ alkylsulfonyl,

(13) optionally substituted C₃-C₈ cycloalkyl,

(14) optionally substituted (C₁-C₆ alkyl)aryl,

(15) optionally substituted C₅-C₈ cycloalkenyl,

(16) C₁-C₅ pefluoroalkyl,

(17) arylsulfonyl optionally substituted with 1 to 3 groups independently selected from C₁-C₅ alkyl, C₁-C₅ perfluoroalkyl, nitro, halogen and cyano,

(18) a 4- to 8-membered heterocycle optionally substituted with 1 to 4 groups independently selected from C₁-C₅ alkyl, C₂-C₅ alkenyl, C₁-C₅ perfluoroalkyl, NR^{g}R^{h}, oxo, thiono, OH, C₁-C₅ alkoxy, C₁-C₅ alkanoyl, C(O)NR^{g}R^{h}, cyano, CO₂H, CO₂-C₁-C₆ alkyl and halogen;

where the substituents on the optionally substituted alkyl, alkenyl, alkynyl, alkanoyl, alkenoyl, alkynoyl, aroyl, aryl, cycloalkanoyl, cycloalkenoyl, alkylsulfonyl, cycloalkyl and cycloalkenyl are from 1 to 10 groups independently selected from OH, C₁-C₆ alkoxy, C₁-C₆ alkyl, C₃-C₇ cycloalkyl, aryl C₁-C₃ alkoxy, NR^{g}R^{h}, CO₂H, CO₂C₁-C₆ alkyl, C(O)NR^{g}R^{h}, NR^{g}C(O)C₁-C₆ alkyl and halogen,

R^{b} is

(1) H,

(2) optionally substituted aryl,

(3) optionally substituted C₁-C₁₀ alkyl,

(4) optionally substituted C₃-C₁₀ alkenyl,

(5) optionally substituted C₃-C₁₀ alkynyl,

(6) optionally substituted C₃-C₁₅ cycloalkyl,

(7) optionally substituted C₀-C₆ alkyl S(O)₂Rⁱ,

(8) optionally substituted C₂-C₆ alkanoyl,

(9) optionally substituted C₅-C₁₀ cycloalkenyl, or

(10) optionally substituted 4- to 8-membered heterocycle;

where the substituents on the optionally substituted aryl, alkyl, alkenyl, cycloalkyl, cycloalkenyl, heterocycle, or alkynyl are from 1 to 10 groups independently selected from

(a) C₁-C₆ alkyl optionally substituted with aryl, hydroxy or amino,

(b) C₁-C₅ perfluoroalkyl;

(c) C₃-C₇ cycloalkyl optionally substituted with 1 to 4 groups independently selected from R^{e},

(d) C₅-C₇ cycloalkenyl,

(e) halogen,

(f) cyano,

(g) OH,

(h) XC₁-C₆ alkyl optionally substituted with amino, hydroxy or aryl optionally substituted with 1,2-methylenedioxy or 1 to 5 groups independently selected from R^{e},

(i) OC(O)C₁-C₅ alkyl,

(j) aryl C₁-C₆ perfluoroalkoxy,

(k) oxo,

(l) SO₂NR^{g}R^{h},

(m)C(O)Rⁱ,

(n) CO₂Rⁱ,

(o) C(O)NR^{g}R^{h},

(p) NR^{g}R^{h},

(q) N(R^{g})CO₂Rⁱ,

(r) N(R^{c})C(S)ORⁱ,

(s) 4- to 8-membered heterocycle optionally substituted with 1 to 5 groups independently selected from R^{e}, and

(t) aryl optionally substituted with 1,2-methylenedioxy or 1 to 5 groups independently selected from R^{e},

R^{c} and R^{d} are independently selected from R^{b}; or

R^{c} and R^{d} together with the N to which they are attached form a 3- to 10-membered ring containing 0 to 2 additional heteroatoms selected from O, S(O)ₘ, and NR^{g}, optionally substituted with 1 to 3 groups independently selected from R^{g}, hydroxy, thiono and oxo;

R^{e} is

(1) halogen,

(2) C₁-C₇ alkyl,

(3) C₁-C₃ perfluoroalkyl,

(4) cyano,

(5) nitro,

(6) RⁱX(CH2)_{v—},

(7) RⁱCO2(CH2)_{v—},

(8) RⁱOCO(CH2)ᵥ,

(9) aryl optionally substituted with from 1 to 3 of halogen, C₁-C₆ alkyl, C₁-C₆ alkoxy, or hydroxy,

(10) SO₂NR^{g}R^{h},

(11) amino, or

(12) oxo;

R^{f} is

(1) C₁-C₄ alkyl,

(2) C₂-C₄ alkenyl,

(3) C₂-C₄ alkynyl,

(4) C₁-C₃-perfluoroalkyl,

(5) NY¹Y²,

(6) NHC(O)C₁-C₅ alkyl,

(7) OH,

(8) X—C₁-C₄ alkyl, or

(9) halogen,

R^{g} and R^{h} are independently

(1) hydrogen,

(2) C₁-C₆ alkyl optionally substituted with hydroxy, amino, C₁-C₅ alkanoyl or CO₂Rⁱ

(3) C₀-C₆aryl optionally substituted with halogen, 1,2-methylene-dioxy, C₁-C₇ alkoxy, C₁-C₇ alkyl or C₁-C₃ perfluoroalkyl,

(4) C(O)OC₁-C₅ alkyl optionally substituted with aryl,

(5) C(O)C₁-C₅ alkyl,

(6) C(O)NY¹Y², or

R^{g} and R^{h} together with the N to which they are attached form a 3- to 7-membered ring containing 0 to 2 additional heteroatoms selected from O, S(O)ₘ, and NRⁱ, optionally substituted with 1 to 3 groups independently selected from halogen, C₁-C₇ alkyl, C₁-C₃ perfluoroalkyl, cyano, nitro, RⁱX(CH₂)_{v—}, RⁱCO₂(CH₂)_{v—}, RⁱOCO(CH₂)ᵥ, aryl optionally substituted with from 1 to 3 of halogen, C₁-C₆ alkyl, C₁-C₆ alkoxy, or hydroxy, and oxo;

Rⁱ is

(1) hydrogen,

(2) C₁-C₃ perfluoroalkyl,

(3) C₁-C₆ alkyl,

(4) optionally substituted aryl C₀-C₆ alkyl, where the aryl substituents are from 1 to 3 groups independently selected from halogen, C₁-C₆ alkyl, C₁-C₆ alkoxy, and hydroxy;

X is O or S(O)ₘ,

Y¹ and Y² are independently hydrogen or C₁-C₅ alkyl,

m is O to 2;and

v is O to 3;or

a pharmaceutically acceptable salt thereof.

In one subset of compounds of formula I, R₆ is the fragment.

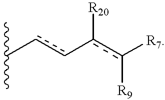

In one embodiment thereof, R₂₀ is hydrogen or C₁-C₅ alkyl, R₇ is CN, CO₂R^{b} or CO₂NR^{c}R^{d}, and R^{g} is other than methyl, and may be for example, hydrogen, ethyl, n-propyl, 3-dihydroxy-1-propyl, 2-oxoethyl, 2-hydroxyethyl, 2-(methylcarbamoyloxy)ethyl, 2-aminoethyl, 2-(ethylcarbamoylamino)ethyl, 2-(ethylamino)ethyl, fluorine, hydroxymethyl, methoxy, n-butyl, allyl, cyano, 4-(1-butenyl), 3-oxopropyl, 3,4-dihydroxybutyl, benzyloxycarbonylamino, trifluoromethyl, acetyl, 2-(ethylcarbamoyloxy)ethyl, 2-(methylcarbamoyloxy)ethyl, 2-(isopropyl-carbamoyloxy)ethyl, 2-(t-butylcarbamoyloxy)ethyl, phenyl, 4-fluorophenyl, methoxycarbonylamino, difluorochloroacetyl, isobutylaminocarbonylmethyl.

In another embodiment thereof R₂₀ is hydrogen or C₁-C₅ alkyl, R₇ is selected from hydrogen, NR^{c}C(O)R^{a}, NR^{c}C(O)NR^{c}R^{d}, NR^{c}C(O)OR^{b}, C(O)R^{a}, P(O)(OR^{a})₂, optionally substituted heterocycle, optionally substituted aryl, CH═NOR^{a}, OSO₂R^{a}, NR^{c}C(O)P(O)(R^{a})₂, NR^{c}C(O)SR^{b}, substituted methyl wherein the substitutents are selected from CO₂R^{b}, C(O)R^{a}, NR^{c}R^{d}, XR^{a}. Aryl are for example, tetrazolyl, thienyl, isoxazolyl, oxazolyl, and thiazolyl; heterocycles are for example, oxazolinyl or thiazolinyl.

In another subset of compounds of formula I, R₆ is the fragment

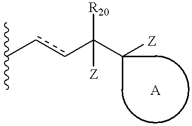

wherein the two Z groups together form a bond across the carbon atoms to which they are attached, and R₂₀ is hydrogen. In one embodiment thereof A

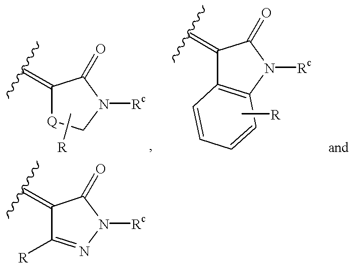

In another subset of compounds of formula I are compounds of formula Ia:

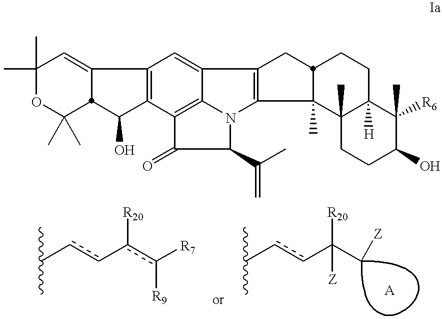

The present invention provides in another aspect pharmaceutical compositions comprising a compound of Formula I and a pharmaceutically acceptable carrier. Such compositions may further comprise one or more other active ingredients such as anthelmintic agents, insect regulators, ecdosyne agonists and fipronil.

The present invention provides in another aspect a method for treating parasitic diseases in a mammal which comprises administering an antiparasitic amount of a compound of Formula I. The treatment may further comprise co-administering one or more other active ingredients such as anthelmintic agents, insect regulators, ecdosyne agonists and fipronil.

“Alkyl” as well as other groups having the prefix “alk”, such as alkoxy, alkanoyl, alkenyl, alkynyl and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains containing at least one unsaturated C—C bond.

The term “cycloalkyl” means carbocycles containing no heteroatoms, and includes mono-, bi- and tricyclic saturated carbocycles, as well as benzofused carbocycles and spirofused carbocycles. Examples of cycloalkyl include cyclopropyl, cyclobutyl, cyclopentyl, cyclohexyl, decahydronaphthalene, adamantane, indanyl, indenyl, fluorenyl, 1,2,3,4-tetrahydronaphalene and the like. Similarly, “cycloalkenyl” means carbocycles containing no heteroatoms and at least one non-aromatic C—C double bond, and include mono-, bi- and tricyclic partially saturated carbocycles, as well as benzofused cycloalkenes. Examples of cycloalkenyl include cyclohexenyl, indenyl, and the like.

The term “halogen” is intended to include the halogen atoms fluorine, chlorine, bromine and iodine.

The term “heterocycle”, unless otherwise specified, means mono- or bicyclic compounds that are saturated or partly unsaturated, as well as benzo- or heteroaromatic ring fused saturated heterocycles or partly unsaturated heterocycles, and containing from 1 to 4 heteroatoms independently selected from oxygen, sulfur and nitrogen. Examples of saturated heterocycles include morpholine, thiomorpholine, piperidine, piperazine, tetrahydropyran, tetrahydrofuran, dioxane, tetrahydrothiophene, oxazolidine, pyrrolidine; examples of partly unsaturated heterocycles include dihydropyran, dihydropyridazine, dihydrofuran, dihydrooxazole, dihydropyrazole, dihydropyridine, dihydropyridazine and the like. Examples of benzo- or heteroaromatic ring fused heterocycle include 2,3-dihydrobenzofuranyl, benzopyranyl, tetrahydroquinoline, tetrahydroisoquinoline, benzomorpholinyl, 1,4-benzodioxanyl, 2,3-dihydrofuro(2,3-b)pyridyl and the like.

The term “aryl” is intended to include mono- and bicyclic aromatic and heteroaromatic rings containing from 0 to 5 heteroatoms independently selected from nitrogen, oxygen and sulfur. The term “aryl” is also meant to include benzofused cycloalkyl, benzofused cycloalkenyl, and benzofused heterocyclic groups, wherein the point of attachment is on the aryl portion. Examples of “aryl” groups include phenyl, pyrrolyl, isoxazolyl, pyrazinyl, pyridinyl, oxazolyl, thiazolyl, imidazolyl, triazolyl, tetrazolyl, furanyl, triazinyl, thienyl, pyrimidinyl, pyridazinyl, pyrazinyl, naphthyl, benzoxazolyl, benzothiazolyl, benzimidazolyl, benzofuranyl, furo(2,3-B)pyridyl, 2,3-dihydrofuro(2,3-b)pyridyl, benzoxazinyl, benzothiophenyl, quinolinyl, indolyl, 2,3-dihydrobenzofuranyl, benzopyranyl, 1,4-benzodioxanyl, indanyl, indenyl, fluorenyl, 1,2,3,4-tetrahydronaphthalene and the like.

Aroyl means arylcarbonyl in which aryl is as defined above.

Examples of NR^{c}R^{d} or NR^{g}R^{h} forming a 3- to 10- membered ring containing 0 to 2 additional heteroatoms selected from O, S(O)ₘ and N are aziridine, azetidine, pyrrolidine, piperidine, thiomorpholine, morpholine, piperazine, octahydroindole, tetrahydroisoquinoline and the like.

The term “optionally substituted” is intended to include both substituted and unsubstituted; thus, for example, optionally substituted aryl could represent a pentafluorophenyl or a phenyl ring.

The term “composition”, as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

Certain of the above defined terms may occur more than once in the above formula and upon such occurrence each term shall be defined independently of the other; thus, for example, OR^{a} at C24 may represent OH and at C7 represent O-acyl.

Compounds described herein contain one or more asymmetric centers and may thus give rise to diastereomers and optical isomers. The present invention is intended to include all possible diastereomers as well as their racemic and resolved, enantiomerically pure forms and all possible geometric isomers. In addition, the present invention includes all pharmaceutically acceptable salts thereof. The term “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

Compounds of the present invention are named based on the trivial name of the parent compound, nodulisporic acid A (compound A), and their position numbers are those as indicated in compound A.

Compounds of the present invention are prepared from two nodulisporic acids (Compounds A and B), which in turn are obtained from the fermentation culture of Nodulisporium sp. MF-5954 (ATCC₇₄₂₄₅). The description of the producing microorganism, the fermentation process, and the isolation and purification of the three nodulisporic acids are disclosed in U.S. Pat. No. 5,399,582, issued Mar. 21, 1995, and Ondeyka, J. G. et al., (J. Am. Chem. Soc. 1997, 119(38), 8809-8816) which is hereby incorporated by reference in its entirety.

The above structural formula is shown without a definitive stereochemistry at certain positions. However, during the the course of the synthetic procedures used to prepare such compounds, or using racemization or epimerization procedures known to those skilled in the art, the products of such procedures can be a mixture of stereoisomers. In particular, the stereoisomers at C7, C24, C1′, C2′, C1″, C2″, C3″, C4″ and C5″ may be oriented in either the alpha- or beta-position, representing such groups oriented below or above the plane of the molecule, respectively. In each such case, and at other positions in the molecule, both the alpha- and beta-configurations are intended to be included within the ambit of this invention.

Compounds of formula I wherein the allyl group at position 2′ is in the epi configuration may be obtained by treatment of the appropriate precursor with a bases such as hydroxide, methoxide, imidazole, triethylamine, potassium hydride, lithium diisopropylamide and the like in protic or aprotic solvents (as appropriate) such as water, methanol, ethanol, methylene chloride, chloroform, tetrahydrofuran, dimethylformamide and the like. The reaction is complete at temperatures from −78° C. to the reflux temperature of the solution in from 15 minutes to 12 hours.

During certain reactions described below, it may be necessary to protect the groups at C24 and C7. With these positions protected, the reactions may be carried out at other positions without affecting the remainder of the molecule. Subsequent to any of the described reactions (vida infra), the protecting group(s) may be removed and the unprotected product isolated. The protecting groups employed at C24 and C7 are those which may be readily synthesized, not significantly affected by the reactions at the other positions, and may be removed without significantly affecting any other functionality of the molecule. One preferred type of protecting group is the tri-substituted silyl group, preferably the tri-loweralkyl silyl group or di-loweralkyl-aryl silyl group. Especially preferred examples are the trimethylsilyl, triethylsilyl, triisopropylsilyl, tert-butyldimethylsilyl and dimethylphenylsilyl groups.

The protected compound may be prepared with the appropriately substituted silyl trifluoromethanesulfonate, BSTFA, hexamethyldisilazane or silyl halide, preferably the silyl chloride. The reaction is carried out in an aprotic solvent such as methylene chloride, benzene, toluene, ethyl acetate, isopropyl acetate, tetrahydrofuran, dimethylformamide and the like. In order to minimize side reactions, there is included in the reaction mixture a base to react with the acid released during the course of the reaction. Preferred bases are amines such as imidazole, pyridine, triethylamine or diisopropylethylamine and the like. The base is required in amounts equimolar to the amount of hydrogen halide liberated, however, generally several equivalents of the amine are employed. The reaction is stirred at from 0° C. to the reflux temperature of the reaction mixture and is complete from 1 to 24 hours.

The silyl group is removed by treatment of the silyl compound with anhydrous pyridine-hydrogen fluoride in tetrahydrofuran or dimethylsulfoxide or with tetraalkylammonium fluoride in tetrahydrofuran. The reaction is complete in from 1 to 24 hours at from o° C. to 50° C. Alternatively, the silyl group may be removed by stirring the silylated compound in lower protic solvents such as methanol, ethanol, isopropanol and the like catalyzed by an acid, preferably a sulfonic acid monohydrate such as para-toluenesulfonic acid, benzenesulfonic acid, pyridinium para-toluenesulfonate or carboxylic acids such as acetic acid, propionic acid, monochloroacetic acid, dichloroacetic acid, trichloroacetic acid and the like. The reaction is complete in 1 to 24 hours at from 0° C. to 50° C.

Protecting groups that may also be suitably used in the preparation of compounds of the present invention may be found in standard textbooks such as Greene and Wutz, Protective Groups in Organic Synthesis, 1991, John Wiley & Sons, Inc.

Compounds of formula I where R₁ and R₂ together represent an oxime, ═NOR^{a}, may be prepared by treating the appropriate oxo analog with H₂NOR^{a} to produce the corresponding oxime. Oxime formation may be accomplished using techniques known to those skilled in the art, including, but not restricted to, the use of H₂NOR^{a} either as the free base or as an acid addition salt such as the HCl salt, or an O-protected hydroxylamine such as O-trialkylsilylhydroxylamine, in a protic solvent such as methanol, ethanol, isopropanol and the like or aprotic solvents such as methylene chloride, chloroform, ethyl acetate, isopropyl acetate, tetrahydrofuran, dimethylformamide, benzene, toluene and the like, as appropriate. The reactions may by catalyzed by the addition of sulfonic acids, carboxylic acids or Lewis acids, including, but not limited to, benzenesulfonic acid monhydrate, para-toluenesulfonic acid monohydrate, acetic acid, zinc chloride and the like.

Similarly, compounds of formula I wherein R₁ and R₂ together represent ═NNR^{c}R^{d} may be prepared by treating the appropriate oxo analog with H₂NNR^{c}R^{d} to give the corresponding hydrazones using conditions directly analogous to those described for oxime formation.

Compounds of formula I wherein R₆ contains CO₂R^{b} may be transesterified by heating the solution in an alcoholic solvent with a Lewis acid catalyst from 50° C. to 200° C., or most preferably 120° C. Suitable alcohols include methanol, ethanol, allyl alcohol, propanol, benzyl alcohol, 2-trimethylsilylethylalcohol and the like. These reactions may also be performed using a co-solvent such as benzene or toluene. Suitable Lewis acids include MgCl₂, MgBr₂, AlCl₃, ZnI₂ and the like, or most preferably, Ti(OiPr)₄. Standard conditions for these reactions are described in Seebach, D. et al., Synthesis (1982), 138-141.

Compounds of formula I wherein one or both of the ___ bonds represent a single bond may be prepared from the corresponding compound wherein ___ ___ is a double bond by conventional hydrogenation procedures. The double bonds may be hydrogenated with any of a variety of standard precious metal hydrogenation catalysts such as Wilkinson's catalyst, Pearlman's catalyst, 1-25% palladium on carbon, 1-25% platinum on carbon and the like. The reaction is generally carried out in a non-reducible solvents (either protic or aprotic) such as methanol, ethanol, isopropanol, tetrahydrofuran, ethyl acetate, isopropyl acetate, benzene, toluene, dimethylformamide and the like. The hydrogen source may be hydrogen gas from 1 to 50 atmospheres of pressure or other hydrogen sources such as ammonium formate, cyclohexene, cyclohexadiene and the like. The reduction also may be carried out using sodium dithionite and sodium bicarbonate in the presence of a phase transfer catalyst, in particular a tetraalkylammonium phase transfer catalyst, and the like. The reactions may be run from 0° C. to 100° C. and are complete in from 5 min to 24 hours.

Compounds of formula I wherein R₂ is OH and R₁ is H may be prepared from the corresponding ketone by treating the appropriate oxo analog with standard reducing agents including, but not restricted to, sodium borohydride, lithium borohydride, lithium aluminum hydride, potassium tri-sec-butyl borohydride, diisobutylaluminum hydride, diborane oxazaborolidines and alkylboranes (both achiral and chiral). These reactions are performed in a manner known to those skilled in the art and are carried out in non-reducible solvents such as methanol, ethanol, diethyl ether, tetrahydrofuran, hexanes, pentane, methylene chloride and the like. The reactions are complete in from 5 minutes to 24 hours at temperatures ranging from −78° C. to 60° C. Compounds of formula I wherein R₂ is OH, R₁ is H and R₆ contains CH₂OH may be obtained by reacting the appropriate carboxylic acid or ester analog (e.g., where R₆ contains CO₂H or CO₂R^{a}) with the more reactive reducing agents as described above, including lithium aluminum hydride, lithium borohydride and the like. Compounds of formula I wherein R₂ and R₁ together are oxo and R₆ contains CH₂OH may be obtained by reacting the appropriate carboxylic acid (e.g., where R₆ contains CO₂H) with less reactive reducing agents such as diborane and the like.

Compounds of formula I wherein R₂ is OH and R₁ is other than H, may be prepared from the corresponding ketone by treating the appropriate oxo analog with a Grignard reagent R₁MgBr, or with a lithium reagent R₁Li. These reactions are performed in a manner known to those skilled in the art and preferably are performed in aprotic solvents such as diethyl ether, tetrahydrofuran, hexanes or pentanes. The reactions are complete in from 5 minutes to 24 hours at temperatures ranging from −78° C. to 60° C.

Compounds of formula I where R₆ contains C(O)N(OR^{b})R^{c} or C(O)NR^{c}R^{d} are prepared from the corresponding carboxylic acid using standard amide-forming reagents known to those skilled in the art. The reaction is carried out using at least one equivalent of an amine nucleophile, HN(OR^{b})R^{c} or HNR^{c}R^{d}, although preferably ten to one hundred equivalents of amine nucleophiles are employed. Amide-forming reagents include, but are not restricted to, dicyclohexyl-carbodiimide, 1-(3-dimethylaminopropyl)-3-ethylcarbodiimide hydrochloride (EDC.HCl), diisopropylcarbodiimide, benzotriazol-1-yloxy-tris(dimethylamino)-phosphonium hexafluorphosphate (BOP), bis(2-oxo-3-oxazolidinyl)phosphinic chloride (BOP-Cl), benzotriazole-1-yl-oxy-tris-pyrrolidino-phosphonium hexafluorophosphate (PyBOP), chloro-tris-pyrrolidino-phosphonium hexafluorophosphate (PyCloP), bromo-tris-pyrrolidino-phosphonium hexafluorophosphate (PyBroP), diphenylphosphoryl azide (DPPA), 2-(1H-benzotriazole-1-yl)-1,1,3,3-tetramethyl-uronium hexafluorophosphate (HBTU), O-benzotriazol-1-yl-N,N,N′,N′-bis(penta-methylene)uronium hexafluorophosphate and 2-chloro-1-methylpyridinium iodide. The amide-forming reactions may be facilitated by the optional addition of N-hydroxybenzotriazole or N-hydroxy-7-aza-benzotriazole. The amidation reaction is generally performed using at least one equivalent (although several equivalents may be employed) of amine bases such as triethylamine, diisopropylethylamine, pyridine, N,N-dimethylaminopyridine and the like. The carboxyl group may be activated for amide bond formation via its corresponding acid chloride or mixed anhydride, using conditions known to those skilled in the art. These amide-forming reactions are carried out in aprotic solvents such as methylene chloride, tetrahydrofuran, diethyl ether, dimethylformamide, N-methylpyrrolidine and the like at −20° C. to 60° C. and are complete in 15 minutes to 24 hours.

Compounds of formula I where R₆ contains CO₂R^{b} are prepared from the corresponding carboxylic acid using standard ester-forming reagents known to those skilled in the art. The esterification reaction is carried out using at least one equivalent of an alcohol, HOR^{b}, although preferably ten to one hundred equivalents of alcohol are used; the esterification also may be carried out using the alcohol as solvent. Esterification reagents include, but are not restricted to, dicyclohexylcarbodiimide, 1-(3-dimethylaminopropyl)-3-ethylcarbodiimide hydrochloride (EDC.HCl), diisopropylcarbodiimide, benzotriazol-1-yloxy-tris(dimethylamino)phosphonium hexafluorphosphate (BOP), bis(2-oxo-3-oxazolidinyl)phosphinic chloride (BOP-Cl), benzotriazole-1-yl-oxy-tris-pyrrolidino-phosphonium hexafluorophosphate (PyBOP), chloro-tris-pyrrolidino-phosphonium hexafluorophosphate (PyCloP), bromo-tris-pyrrolidino-phosphonium hexafluorophosphate (PyBroP), diphenylphosphoryl azide (DPPA), 2-(1H-benzotriazole-1-yl)-1,1,3,3-tetramethyluronium hexafluorophosphate (HBTU), O-benzotriazol-1-yl-N,N,N′,N′-bis(pentamethylene)uronium hexafluorophosphate and 2-chloro-1-methyl-pyridinium iodide. The ester-forming reactions may be facilitated by the optional addition of N-hydroxybenzotriazole, N-hydroxy-7-aza-benzotriazole, 4-(N,N-dimethylamino)pyridine or 4-pyrrolidinopyridine. The reaction is generally performed using at least one equivalent (although several equivalents may be employed) of amine bases such as triethylamine, diisopropylethylamine, pyridine and the like. The carboxyl group may be activated for ester bond formation via its corresponding acid chloride or mixed anhydride, using conditions known to those skilled in the art. These ester-forming reactions are carried out in aprotic solvents such as methylene chloride, tetrahydrofuran, diethyl ether, dimethylformamide, N-methylpyrrolidine and the like at temperatures ranging from −20° C. to 60° C. and are complete in 15 minutes to 24 hours.

Compounds of formula I wherein one or more of R₂, R₃, R₄, and R₉ is OR^{a}, OCO₂R^{b} or OC(O)NR^{c}R^{d}, and/or where R⁹ is CH₂OR^{a}, CH₂OCO₂R^{b} or CH₂OC(O)NRcRd may be prepared using known methods for acylation, sulfonylation and alkylation of alcohols. Thus, acylation may be accomplished using reagents such as acid anhydrides, acid chlorides, chloroformates, carbamoyl chlorides, isocyanates and amine bases according to general procedures known to those skilled in the art. Sulfonylations may be carried out using sulfonylchlorides or sulfonic anhydrides. The acylation and sulfonylation reactions may be carried out in aprotic solvents such as methylene chloride, chloroform, pyridine, benzene, toluene and the like. The acylation and sulfonylation reactions are complete in from 15 minutes to 24 hours at temperatures ranging from −20° C. to 80° C. The degree of acylation, sulfonylation and alkylation will depend on the amount of the reagents used. Thus, for example, using one equivalent of an acylating reagent and one equivalent of nodulisporic acid results in a product mixture containg 4- and 20-acylated nodulisporic acid; such a mixture may be separated by conventional techniques such as chromatography.

Compounds of formula I wherein one or more of R₂, R₃, R₄ is OR^{a} and/or where R⁶ contains CH₂OR^{a}, may be prepared using methods known to those skilled in the art for the alkylation of alcohols. Thus, alkylation may be accomplished using reagents including, but not restricted to, halides IR^{a}, BrR^{a}, ClR^{a}, diazo reagents N₂R^{a}, trichloroacetimidates R^{a}OC(NH)CCl₃, sulfates R^{a}OSO₂Me, R^{a}OSO₂CF₃, and the like. The alkylation reactions may be facilitated by the addition of acid, base or Lewis acids, as appropriate. The reactions are performed in aprotic solvents such as methylene chloride, chloroform, tetrahydrofuran, benzene, toluene, dimethylformamide, N-methyl-pyrrolidine, dimethyl sulfoxide, hexamethylphosphoramide and are complete at from 0° C. to the reflux temperature of the solution from 15 minutes to 48 hours.

The 3″ -aldehyde (Compound II) may be prepared as described in Schemes I and II. Thus, compound A or compound B may be treated with potassium permanganate under conditions known to those skilled in the art to yield the aldehyde product, Compound II. The potassium pernanganate may be used stoichiometrically or in excess and in the presence of a solid support including but not restricted to, Celite, basic alumina, neutral alumina, acidic alumina, silica gel, clays and the like. Sodium permanganate or tetraalkylammonium permanganate (either preformed or generated in situ from a tetraalkylamonium salt and potassium permanganate) may be substituted for potassium permanganate. Suitable tetraalkylammonium salts include, but are not restricted to (n-Bu)₄NX, (PhCH₂)₃NMeX, (n-heptyl)₄NX, (PhCH2)N(n-Bu)₃X, (n-dodecyl)₃NMeX, Adogen 464 and the like and where X═HO, SO₄, PF₆ and the like. The reaction to form Compound II may be performed in a variety of solvents or mixtures of solvents. These include both protic and aprotic solvents such as water, methylene chloride, chloroform, dichloroethane, methanol, ethanol, tert-butanol, ether, tetrahydrofuran, benzene, pyridine, acetone and the like. The reactions may be performed at from −78° C. to 80° C. and are complete in from 5 minutes to 24 hours.

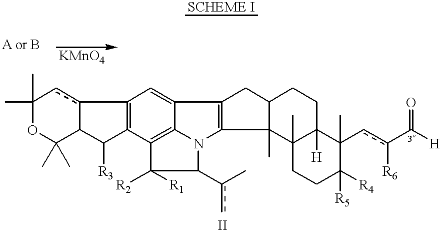

Compound II, the 3″ -aldehyde, may also be produced by treating Compound III with osmium tetroxide under conditions known to those skilled in the art as shown in Scheme II below. Also produced during this reaction is the diol product IV. Mono- and disubstituted amides of compound III may be used in this reaction. These include, but are not restricted to, monosubstituted amides such as N-methyl, N-ethyl, N-propyl, N-butyl, N-tert-butyl, N-phenyl and the like or disubstituted amides such as N,N-dimethyl, N,N-diethyl, N-methyl-N-ethyl, N-methyl-N-phenyl and the like. Osmium tetroxide may be used either stoichiometrically or catalytically in the presence of an ocidant, including, but not restricted to, morpholine N-oxide, trimethylamine N-oxide, hydrogen peroxide, tert-butyl hydroperoxide and the like. The dihydroxylation reactions may be performed in a variety of solvents or mixtures of solvents. These include both protic and aprotic solvents such as water, methanol, ethanol, tert-butanol, ether, tetrahydrofuran, benzene, pyridine, acetone and the like. The reactions may be performed at from −78° C. to 80° C. and are complete in from 5 minutes to 24 hours. Diol product IV may be converted into 3″ -aldehyde II by treatment with an oxidizing agent, including, but restricted to, NaIO₄, HIO₄, MnO₂, Amberlite 904-NaIO₄ and the like, or preferably Pb(OAc)₄. These oxidative cleavage reactions may be performed in a variety of solvents or mixtures of solvents. These include both protic and aprotic solvents such as water, methanol, ethanol, tert-butanol, ether, tetrahydrofuran, benzene, pyridine, acetone and the like. The reactions may be performed at from −78° C. to 80° C. and are complete in from 5 minutes to 24 hours.

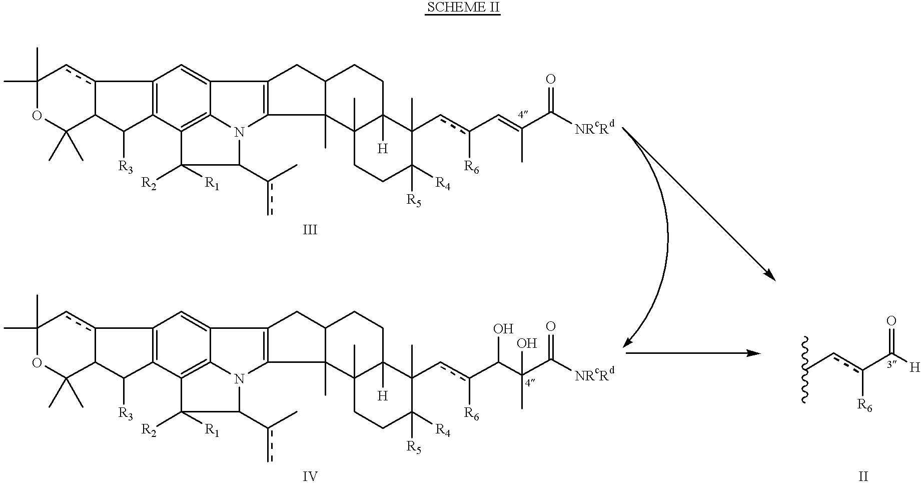

Compounds of formula V may be prepared by reacting Compound II with appropriate olefin-forming reaction conditions known to those skilled in the art as shown in Scheme III below. Reagents for these reactions include, but are not restricted to, the use of stabilized and unstabilized Wittig reagents, Horner-Emmons reagents, Tebbe reagent, Petassis reagent, aldol reactions, Knoevenagel reactions, Peterson olefinations and the like. Starting Compound II may have its C7- and C24-hydroxyl groups protected with silyl protecting groups. The compounds of formula I shown below may be acyclic or cyclic, depending on the chain-extending reagents utilized. Alternatively, compounds of formula V may be acyclic but further modified to yield cyclic products. The olefination reactions may be performed in a variety of solvents or mixtures of solvents. These include both protic and aprotic solvents such as water, methanol, ethanol, tert-butanol, ether, acetonitrile, tetrahydrofuran, methylene chloride, chloroform, 1,2-dichloroethane, benzene, toluene, pyridine, acetone and the like. Suitable bases include, but are not limited to, NaOH, KOH, NaOEt, KOtBu, LDA, LHMDS, NaHMDS, KHMDS, pyridine, piperidine, morpholine, lutidine, DMAP, DBU and the like. The reactions may be performed at from −78° C. to 80° C. and are complete in from 5 minutes to 24 hours. The compounds of formula V may be further elaborated by Stille, Heck and Suzuki couplings where R₆ contains OTf, B(OH)₂, Sn(n-Bu)₃, SnMe₃, OP(O)(OPh)₂, I, Br, or Cl.

Wittig reagents may be readily prepared by reacting Ph₃P with an appropriate halide under conditions known to those skilled in the art, such as those described by Ikuta, H. et al. (J. Med. Chem. 1987, 30, 1995-1998) or Larock, R. C. (Comprehensive Organic Transformations, VCH Publishers: Inc. New York, N.Y., 1989, Chapter 4). Alternatively, stabilized Wittig reagents may be prepared as described by Bestmann, H. J. and Schulz, H. (Chem. Ber. 1964, 97, 11) wherein an appropriate unstabilized Wittig reagent Ph₃P═C(R^{a})₂ is reacted with a suitable chloroformate to yield Ph₃P═C(R^{a})₂CO₂R^{b} or as described by the conjugate addition of Ph₃P to N—R^{c} substituted maleimides.

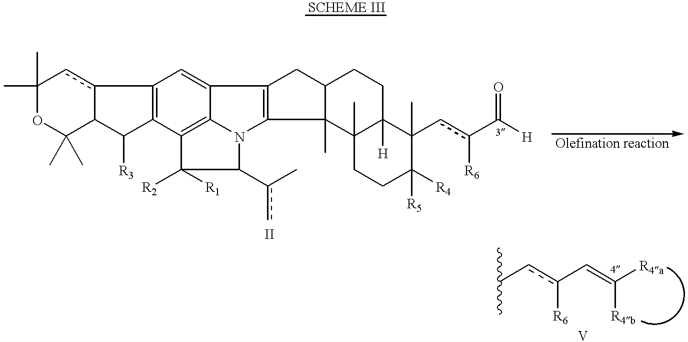

Compounds of formula VI may be prepared as illustrated in Scheme IV by the addition of an appropriate nucleophile to the 3″ -aldehyde of compound II. The C7 and C24 hydroxyls of compound II may be optionally protected with R₃Si groups as previously described. The nucleophilic addition reaction may produce a mixture of stereoisomers at 3″. Suitable nucleophiles include, but are not restricted to, Grignard reagents and organolithium, organocuprates organozinc reagents, organosodium reagents, organopotassium reagents and organocerium reagents and the like. These reagents include, but are not restricted to, MeMgBr, EtLi, PhMgCl, (nPr)₂CuMgI, H₂C═CHMgBr, 2-furfuryl lithium, BrZnCH₂CO₂Me, NaCH₂C(O)Ph(4-Br) and the like. Suitable solvents, or mixtures of solvents for this reaction include, but are not restricted to, toluene, tetrahydrofuran, hexanes, dioxane, 1,2-dimethoxyethane, DMSO, HMPA, DMPU and the like or most preferably diethyl ether. The reactions proceed at from −100° C. to 80° C. and are complete in from 5 min to 12 h. Oxidation of the newly formed 3″ -hydroxyl to form the corresponding 3″ -ketone may be accomplished using reagents known to those skilled in the art. These reagents include, but are not restricted to TPAP, Dess-Martin reagent, Mn(OAc)₂, CuCl, SeO₂, NaOCl/HOAc, DMSO/Ac₂O, DDQ, and the like or most preferably MnO₂. Suitable solvents for this oxidation reaction include, but are not restricted to, EtOAc, CHCl₃, benzene, toluene, THF, and the like or most preferably CH₂Cl₂. Compounds of formula VI may be prepared by reacting the intermediate 3″ -ketone thus prepared with appropriate olefin-forming reaction conditions known to those skilled in the art as described previously.

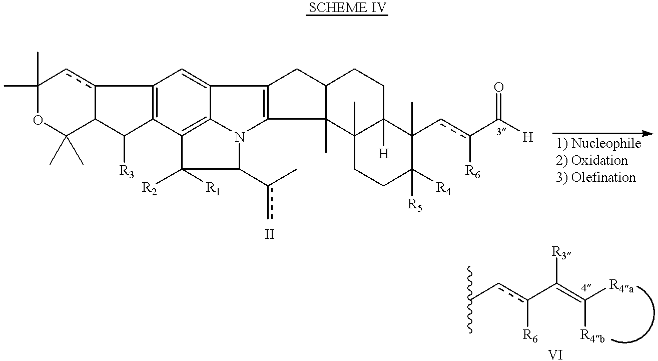

Compounds of formula V (or formula VI) where R_{4″} a (or b) is CO₂R^{b} are useful as intermediates in the preparation of certain compounds of formula V (or formula VI) where R_{4″} a (or b) is C(O)NRcRd. The esters where R4 a (or b) is CO₂R^{b} may be hydrolyzed by treatment with hydroxide or ammonium hydroxide in a protic solvent such as methanol, ethanol, water, tetrahydrofuran/water or dimethylformamide/water and the like at from 0° C. to the reflux temperature of the solution. Alternatively, the resultant esters may be transesterified by treatment with a Lewis acid, including, but not restricted to, magnesium chloride, magnesium bromide, aluminum chloride, zinc chloride, Otera's catalyst, and the like, or preferably titanium tetra-isopropoxide in a protic solvent such as methanol, ethanol, isopropanol, 2-trimethylsilylethyl alcohol and the like, or preferably allyl alcohol. The transesterification reactions are complete in from 1 to 24 hours at 0° C. to the reflux temperature of the solution, preferably 110° C. The allyl ester (e.g. Rb═—CH₂CH═CH₂) may be removed by treatment with Pd° using conditions known to those skilled in the are to generate the free carboxylic acid (e.g. Rb═H). Pd° reagents include, but are not restricted to, PdCl₂(PPh₃)₂, Pd(OAc)₂(PPh₃)₂, PdCl₂(PhCN)₂, Pd(OAc)₂, PdCl₂(P(o-tolyl)₃)₂, PdCl₂(DDPF), Pd₂(dba)₃, and the like, or preferably Pd(PPh₃)₄. Amides (where R_{4′} a (or b) is C(O)NR^{c}R^{d}) are prepared as described (vide supra) from the corresponding carboxylic acids.

Compounds of formula VIII where R_{4″} a(or b) contains NR^{c}C(O)NR^{c}R^{d} (compond VIIIa), NR^{c}CO₂R^{b} (compond VIIIb), NR^{c}C(O)SR^{a} (compond VIIIC), or NR^{c}C(O)R^{a} (compond VIIId), may be prepared from the corresponding carboxylic acids as shown in Scheme V. Thus, compounds of formula V where R_{4″} a(or b) is CO₂His treated with diphenylphosphoryl azide to provide the acyl azide VIIa. Heating of compound VIIa in an aprotic solvent such as benzene, toluene, dimethylformamide and the like results in a rearrangement yielding compound VIIb, an isocyanate. The isocyanate-forming reactions may be performed from 0° C. to 120° C., preferably at 80° C., and are complete in 15 min to 24 hours.

Compounds of formula VIIIa may be prepared when compounds of formula VIIb are reacted with an appropriate amine HNR^{c}R^{d} in an aprotic solvent such as methylene chloride, tetrahydrofuran, dimethylformamide, dimethylsulfoxide, benzene, toluene and the like. The urea-forming reactions may be performed from 0° C. to 100° C. and are complete in 15 minutes to 24 hours.

Compounds of formula VIIb may be reacted in an aprotic solvent such as benzene, toluene, methylene chloride, 1,2-dichloroethylene, dimethylformamide and the like, with an alcohol R^{b}OH, such as methanol, ethanol, benzyl alcohol, 2-trimethylsilylethanol, 2,2,2-trichloroethanol, methyl glyocolate, phenol and the like to yield carbamates of formula VIIIb. Similarly, compounds of formula VIIIc may be prepared by substituting HSR^{b} for HOR^{b} in the reaction. The addition of one or more equivalents of an amine base such as triethylamine, diisopropylethylamine, pyridine and the like may be employed to accelerate carbamate formation. The carbamate-forming reactions may be performed from 0° C. to 100° C. and are complete in 15 minutes to 24 hours.

Compounds of formula VIIId may be prepared by treatment of compounds of formula VIIb with R^{a}MgI, R^{a}MgCl, R^{a}Li, (R^{a})₂CuLi or preferably R^{a}MgBr, as illustrated below. Compounds of formula VIIb may be reacted in an aprotic solvent, or mixture of solvents, such as including, but not restricted to, dioxane, pentane, hexane, DMSO, HMPA, or NMP, and the like, or preferably tetrahydrofuran. The reactions may be performed from −78° C. to 100° C. and is complete in from 5 minutes to 12 hours.

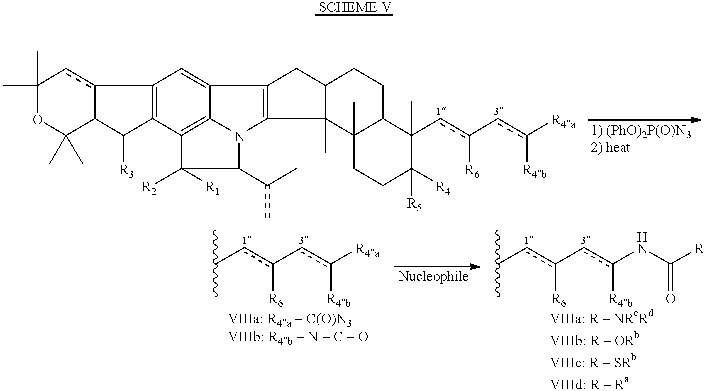

Compounds of formula I may be further modified as illustrated in Scheme VI. For instance, the allyl ester at C5″ of compounds of formula IX may be converted into the corresponding 5″ -amide (compounds of formula Xa) as shown and the propenyl group at C4″ modified by treatment with osmium tetroxide (step 3) to generate the corresponding diol (compounds of formula Xb). The diol of formula Xb may be subjected to oxidative cleavage (step 4) with lead tetraacetate to form aldehydes of formula Xc. The aldehyde of compounds of formula Xc may be reduced to generate the corresponding intermediate alcohol (not shown) which may be acylated (steps 5 or 6) to form compounds of formula Xd or Xe. The aldehyde of Xc may be converted into an amine of formula Xf (step 7) via reductive amination with HNR^{c}R^{d} and an appropriate reducing agent, and the resultant amine (Xf where R^{d} is hydrogen) may be optionally acylated to form compounds of formula Xg (step 8) or sulfonylated. In addition, the aldehyde of Xc may be reacted with chain extending olefinating reagents such as Wittig reagents, Horner-Emmons reagents and the like as described previously (compounds not shown).

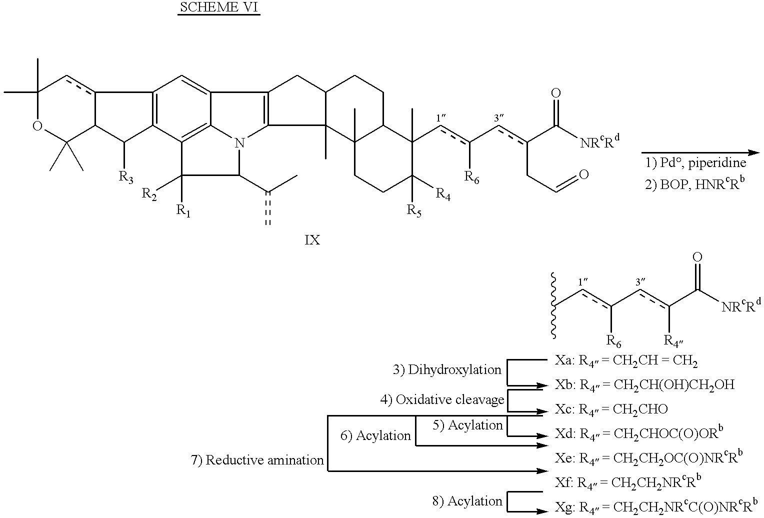

Alternatively, the aldehyde of Xc may be treated with an acid such as PPTS in an alcoholic solvent such as methanol, ethanol, n-propanol and the like to generate cyclic compounds of formula XIa and XIb as illustrated in Scheme VII.

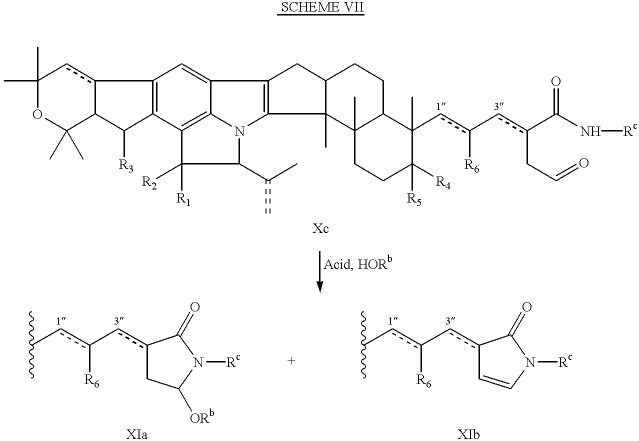

Compounds of formula XIIIa may be prepared using the Passerrini reaction wherein compounds of formula XII are treated with a carboxylic acid, an isonitrile in a protic solvent as shown in Scheme VIII. Suitable isonitriles for the Passerrini reaction, include, but are not limited to, methyl, ethyl, isopropyl, tert-butyl, cyclohexenyl, benzyl and ethyl isonitriloacetate and the like. The Passerrini reaction may be performed in protic solvents or mixtures of solvents, including, but not limited to, methanol, ethanol, isopropanol, tert-butanol or water as well as aprotic solvents, including, but not limited to methylene chloride, DMSO, DMF, NMP, THF, chloroform, toluene and the like at temperatures from 0° C. to 80° C. but most preferably at room temperature. Suitable carboxylic acids include, but are not limited to acetic acid, proprionic acid, formic acid, alpha-chloroacetic acid, alpha-methoxyacetic acid, butyric acid, benzoic acid and the like. Substitution of a sulfonic acid and an amine base for the carboxylic acid leads to the formation of compounds of formula XIIIb. Suitable sulfonic acids include, but are not limited to benzene sulfonic acid or methane sulfonic acid, and the like or most preferably toluene sulfonic acid. Suitable amine bases include, but are not limited to, Et3N, DIEA, DBU, lutidine, imidazole, quinoline and the like, or most preferably pyridine. Suitable protic solvents for the formation of compound XMb include, but are not limited to, water, methanol, ethanol, n-propanol, butanol, isopropanol, allyl alcohol, tert-butanol, 2,2,2-trifluoroethanol, phenol, benzyl alcohol, ethylene glycol, methyl glycolate and the like.

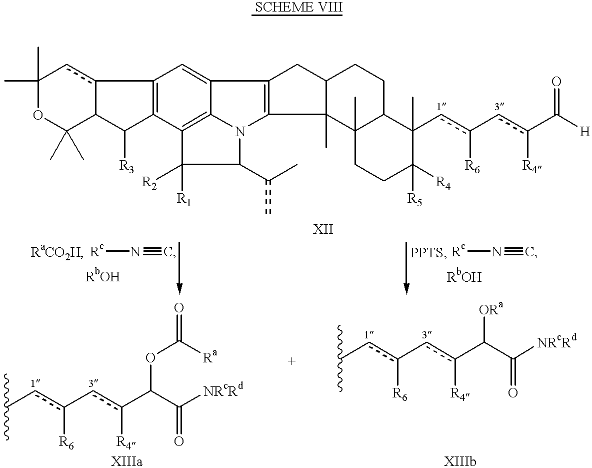

The instant compounds are potent endo- and ecto-antiparasitic agents, particularly against helminths, ectoparasites, insects, and acarids, infecting man, animals and plants, thus having utility in human and animal health, agriculture and pest control in household and commercial areas.

The disease or group of diseases described generally as helminthiasis is due to infection of an animal host with parasitic worms known as helminths. Helminthiasis is a prevalent and serious economic problem in domesticated animals such as swine, sheep, horses, cattle, goats, dogs, cats, fish, buffalo, camels, llamas, reindeer, laboratory animals, furbearing animals, zoo animals and exotic species and poultry. Among the helminths, the group of worms described as nematodes causes widespread and often times serious infection in various species of animals. The most common genera of nematodes infecting the animals referred to above are Haemonchus, Trichostrongylus, Ostertagia, Nematodirus, Cooperia, Ascaris, Bunostomum, Oesophagostomum, Chabertia, Trichuris, Strongylus, Trichonema, Dictyocaulus, Capillaria, Habronema, Druschia, Heterakis, Toxocara, Ascaridia, Oxyuris, Ancylostoma, Uncinaria, Toxascaris and Parascaris. Certain of these, such as Nematodirus, Cooperia, and Oesophagostomum attack primarily the intestinal tract while others, such as Haemonchus and Ostertagia, are more prevalent in the stomach while still others such as Dictyocaulus are found in the lungs. Still other parasites may be located in other tissues and organs of the body such as the heart and blood vessels, subcutaneous and lymphatic tissue and the like. The parasitic infections known as helminthiases lead to anemia, malnutrition, weakness, weight loss, severe damage to the walls of the intestinal tract and other tissues and organs and, if left untreated, may result in death of the infected host. The compounds of this invention have activity against these parasites, and in addition are also active against Dirofilaria in dogs and cats, Nematospiroides, Syphacia, Aspiculuris in rodents, arthropod ectoparasites of animals and birds such as ticks, mites such as scabies lice, fleas, blowflies, and other biting insects in domesticated animals and poultry, such as Tenophalides, Ixodes, Psoroptes, and Hemotobia, in sheep Lucilia sp., biting insects and such migrating dipterous larvae as Hypoderma sp. in cattle, Gastrophilus in horses, and Cuterebra sp. in rodents and nuisance flies including blood feeding flies and filth flies.

The instant compounds are also useful against parasites which infect humans. The most common genera of parasites of the gastro-intestinal tract of man are Ancylostoma, Necator, Ascaris, Strongyloides, Trichinella, Capillaria, Trichuris, and Enterobius. Other medically important genera of parasites which are found in the blood or other tissues and organs outside the gastrointestinal tract are the filiarial worms such as Wuchereria, Brugia, Onchocerca and Loa, Dracunuculus and extra intestinal stages of the intestinal worms Strongyloides and Trichinella. The compounds are also of value against arthropods parasitizing man, biting insects and other dipterous pests causing annoyance to man.

The compounds are also active against household pests such as the cockroach, Blatella sp., clothes moth, Tineola sp., carpet beetle, Attagenus sp., the housefly Musca domestica as well as fleas, house dust mites, termites and ants.

The compounds of this invention are also useful in combatting agricultural pests that inflict damage upon crops while they are growing or while in storage. The compounds are applied using known techniques as sprays, dusts, emulsions and the like, to the growing or stored crops to effect protection from such agricultural pests.

The compounds are also useful against insect pests of stored grains such as Tribolium sp., Tenebrio sp. and of agricultural plants such as aphids, (Acyrthiosiphon sp.); against migratory orthopterans such as locusts and immature stages of insects living on plant tissue. The compounds are useful as a nematocide for the control of soil nematodes and plant parasites such as Meloidogyne sp. which may be of importance in agriculture. The compounds are also highly useful in treating acreage infested with fire ant nests. The compounds are scattered above the infested area in low levels in bait formulations which are brought back to the nest. In addition to a direct-but-slow onset toxic effect on the fire ants, the compound has a long-term effect on the nest by sterilizing the queen which effectively destroys the nest.

The compounds of this invention may be administered in formulations wherein the active compound is intimately admixed with one or more inert ingredients and optionally including one or more additional active ingredients. The compounds may be used in any composition known to those skilled in the art for administration to humans and animals, for application to plants and for premise and area application to control household pests in either a residential or commercial setting. For application to humans and animals to control internal and external parasites, oral formulations, in solid or liquid or parenteral liquid, implant or depot injection forms may be used. For topical application dip, spray, powder, dust, pour-on, spot-on, jetting fluid, shampoos, collar, tag or harness, may be used. For agricultural premise or area application, liquid spray, powders, dust, or bait forms may be used. In addition “feed-through” forms may be used to control nuisance flies that feed or breed in animal waste. The compounds are formulated, such as by encapsulation, to lease a residue of active agent in the animal waste which controls filth flies or other arthropod pests.

Accordingly, the present invention provides a method for the treatment or prevention of diseases caused by parasites which comprises administering to a host in need of such treatment or prevention an antiparasitic effective amount of a compound of Formula I. The parasites may be, for example, arthropod parasites such as ticks, lice, fleas, mites and other biting arthropods in domesticated animals and poultry. The parasites also include helminths such as those mentioned above.

Compounds of formula I are effective in treatment of parasitic diseases that occur in other animals including humans. The optimum amount to be employed for best results will, of course, depend upon the particular compound employed, the species of animal to be treated and the type and severity of parasitic infection or infestation. Generally good results are obtained with our novel compounds by the oral administration of from about 0.001 to 500 mg per kg of animal body weight, such total dose being given at one time or in divided doses over a relatively short period of time such as 1-5 days. With the preferred compounds of the invention, excellent control of such parasites is obtained in animals by administering from about 0.025 to 100 mg per kg of body weight in a single dose. Repeat treatments are given as required to combat re-infections and are dependent upon the species of parasite and the husbandry techniques being employed. Repeat treatments may be given daily, weekly, biweekly, monthly, or longer for example up to six months, or any combination thereof, as required. The techniques for administering these materials to animals are known to those skilled in the veterinary field.

Compounds of formula I may be co-administered or used in combination with one or more other agents to the host. Co-administration or combination use includes administering all active ingredients in one formulation, for example a tablet, capsule, feed stuff, or liquid containing a compound of formula I and one or more said other agents; administering each ingredient in a separate formulation; and combinations thereof. When one or more of a compound of formula I or said other agent(s) is contained in a separate formulation, any order of administration as well as any interval between the administration of the active ingredients are within the meaning of co-administration or combination use.

Agents that may be co-administered or used in combination with compounds of formula I include any that are used in the treatment or prevention of human or animal diseases or conditions, or used in agricultural applications, or for pest control. In a preferred embodiment, the co-administered agents are used in veterinary medicine, particularly those used in domesticated animals such as dogs and cats or other companion animals. Examples of other agents that may be co-administered with compounds of formula I are provided below. It is to be understood that the specific agents enumerated are illustrative only, and are not meant to be restrictive in any manner.

Accordingly, compounds of the present invention may be co-administered or used in combination with anthelmintic agents. These anthelmintic agents are meant to include, but not be restricted to, compounds selected from the avermectin and milbemycin class of compounds such as ivermectin, avermectin, abamectin, emamectin, eprinamectin, doramectin, milbemycin derivatives described in EPO 357460, EPO 444964 and EPO 594291, moxidectin, Interceptor™ and nemadectin. Additional anthelmintic agents include the benzimidazoles such as thiabendazole, cambendazole, parbendazole, oxibendazole, mebendazole, flubendazole, fenbendazole, oxfendazole, albendazole, cyclobendazole, febantel, thiophanate and the like. Additional anthelmintic agents include imidazothiazoles and tetrahydropyrimidines such as tetramisole-levamisole, butamisole, pyrantel, pamoate, oxantel or morantel.

Compounds of this invention may be co-administered or used in combination with fipronil (FRONTLINE™); or with an insect growth regulator with molt inhibiting activity such as lufenuron (PROGRAM™ or SENTINEL™) and the like; or with ecdysone agonists such as tebufenozide and the like, which induces premature molt and causes feeding to cease; or with imidacloprid (ADVANTAGE™).

Compounds of this invention may be co-administered or used in combination with avermectin or milbemycin or doramectin derivatives such as those described in U.S. Pat. No. 5,015,630, WO 94/15944, WO95/22552, including selamectin (REVOLUTION™).

Compounds of this invention may be co-administered or used in combination with cyclic depsipeptides that exhibit anthelmintic efficacy such as those described in WO96/11945, WO93119053, WO 93/25543, EP 626375, EP 382173, WO 94/19334, EP 382173 and EP 503538.

Compounds of this invention may be used in combination or be co-administered with derivatives and analogs of the general class of dioxomorpholine antiparasitic and anthelmintic agents as illustrated by WO 9615121; or with pyrethroids or organophosphates or insecticidal carbamates, such as those described in “Chemotherapy of Parasitic Diseases”, Campbell, W. C. and Rew, R. S, Eds., 1986; or with derivatives and analogs of the general class of paraherquamide and macfortine anthelmintic agents.

The co-administered compounds are given via routes, and in doses, that are customarily used for those compounds.

Compounds of formula I may be administered orally in a unit dosage form such as a capsule, bolus or tablet including chewable tablet, or as a liquid drench where used as an anthelmintic in mammals. The drench is normally a solution, suspension or dispersion of the active ingredient usually in water together with a suspending agent such as bentonite and a wetting agent or like excipient. Generally, the drenches also contain an antifoaming agent. Drench formulations generally contain from about 0.001 to 0.5% by weight of the active compound. Preferred drench formulations may contain from 0.01 to 0.1% by weight. The capsules and boluses comprise the active ingredient admixed with a carrier vehicle such as starch, talc, magnesium stearate, or di-calcium phosphate.

Where it is desired to administer the instant compounds in a dry, solid unit dosage form, capsules, boluses or tablets containing the desired amount of active compound usually are employed. These dosage forms are prepared by intimately and uniformly mixing the active ingredient with suitable finely divided diluents, fillers, disintegrating agents, and/or binders such as starch, lactose, talc, magnesium stearate, vegetable gums and the like. Such unit dosage formulations may be varied widely with respect to their total weight and content of the antiparasitic agent depending upon factors such as the type of host animal to be treated, the severity and type of infection and the weight of the host.

When the active compound is to be administered via an animal feedstuff, it is intimately dispersed in the feed or used as a top dressing or in the form of pellets or liquid which may then be added to the finished feed or optionally fed separately. Alternatively, feed based individual dosage forms may be used such as a chewable treat. Alternatively, the antiparasitic compounds of this invention may be administered to animals parenterally, for example, by intraruminal, intramuscular, intravascular, intratracheal, or subcutaneous injection in which the active ingredient is dissolved or dispersed in a liquid carrier vehicle. For parenteral administration, the active material is suitably admixed with an acceptable vehicle, preferably of the vegetable oil variety such as peanut oil, cotton seed oil and the like. Other parenteral vehicles such as organic preparation using solketal, glycerol formal, propylene glycol, and aqueous parenteral formulations are also used. The active compound or compounds are dissolved or suspended in the parenteral formulation for administration; such formulations generally contain from 0.0005 to 5% by weight of the active compound.

When the compounds described herein are administered as a component of the feed of the animals, or dissolved or suspended in the drinking water, compositions are provided in which the active compound or compounds are intimately dispersed in an inert carrier or diluent. By inert carrier is meant one that will not react with the antiparasitic agent and one that may be administered safely to animals. Preferably, a carrier for feed administration is one that is, or may be, an ingredient of the animal ration.

Suitable compositions include feed premixes or supplements in which the active ingredient is present in relatively large amounts and which are suitable for direct feeding to the animal or for addition to the feed either directly or after an intermediate dilution or blending step. Typical carriers or diluents suitable for such compositions include, for example, distillers' dried grains, corn meal, citrus meal, fermentation residues, ground oyster shells, wheat shorts, molasses solubles, corn cob meal, edible bean mill feed, soya grits, crushed limestone and the like. The active compounds are intimately dispersed throughout the carrier by methods such as grinding, stirring, milling or tumbling. Compositions containing from about 0.005 to 50% weight of the active compound are particularly suitable as feed premixes. Feed supplements, which are fed directly to the animal, contain from about 0.0002 to 0.3% by weight of the active compounds.

Such supplements are added to the animal feed in an amount to give the finished feed the concentration of active compound desired for the treatment and control of parasitic diseases. Although the desired concentration of active compound will vary depending upon the factors previously mentioned as well as upon the particular compound employed, the compounds of this invention are usually fed at concentrations of between 0.00001 to 10% in the feed in order to achieve the desired anti-parasitic result.

In using the compounds of this invention, the individual compounds may be prepared and used in that form. Alternatively, mixtures of the individual compounds may be used, or they may be combined with other active compounds not related to the compounds of this invention.

Also included in the present invention are pharmaceutical compositions comprising a compound of formula I and a pharmaceutically acceptable carrier. The pharmaceutical compositions of the present invention may further comprise a second active ingredient such as those described above for co-administration. The preferred second ingredient is selected from an anthelmintic agent, fipronil, imidocloprid, an insect growth regulator, or a ecdysone agonist. Said second ingredient is preferably selected from the group consisting of ivermectin, avermectin 5-oxime, abamectin, emamectin, eprinamectin, doramectin, doramectin monosaccharide 5-oximes, fulladectin, milbemycin, milbemycin 5-oxime, moxidectin, Interceptor™, nemadectin, imidacloprid, fipronil, lufenuron, thiabendazole, cambendazole, parbendazole, oxibendazole, mebendazole, flubendazole, fenbendazole, oxfendazole, albendazole, cyclobendazole, febantel, thiophanate, tetramisole-levamisole, butamisole, pyrantel, pamoate, oxantel and morantel.

Preparation of Intermediates

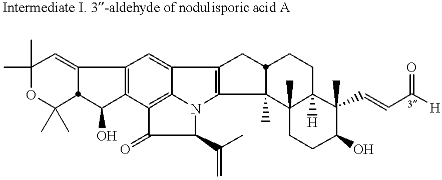

(a) Synthesis from Nodulisporic acid A:

To KMnO₄ (3 g) at 25° C. was added water (5 mL). The KMnO₄ solution was cooled to 0° C. and Al₂O₃ (weakly acidic, 10.8 g) was added and stirred for 5 min until thoroughly mixed. A solution of nodulisporic acid A (3 g) in CH₂Cl₂ (300 mL) was added dropwise via an addition funnel over 20 min. The solution was aged for an additional 20 min at 0° C. then at 25° C. for 90 min. The solution was filtered through a 3 inch pad of Celite using CH₂Cl₂ as eluant followed by EtOAc. The solvents were removed under reduced pressure at ambient temperature to yield pure title compound (2.234 g, 82%) without any additional purification.

(b) Synthesis from N-tert-butyl Nodulisporamide

To N-tert-butyl nodulisporamide A (50 mg) in CH₂Cl₂ (2 mL) at 25° C. was added N-methylmorpholine N-oxide (50 mg) followed by 0.024 M Os04 in water (0.31 mL). After aging the solution for 16 hr, TLC showed the presence of the desired compound and the R,R- and S,S-3″,4″ -diols of N-tert-butyl nodulisporamide A. Intermediate 1 (10.5 mg) and the diols (36 mg) were isolated in pure form by PTLC on silica gel using 2:1 EtOAc:hexanes as eluant. The R,R- and S,S-diols were combined. To a mixture of diols (10 mg) in acetone (0.9 mL) at 25° C. was added NaIO₄ (25 mg) and the solution was allowed to age for 12 h. The solution was poured into saturated aqueous NaHCO₃, extracted with EtOAc and dried (Na₂SO₄). Pure Intermediate 1 (7 mg) was obtained following PTLC on silica gel using 1/1 hexanes/EtOAc as eluant.

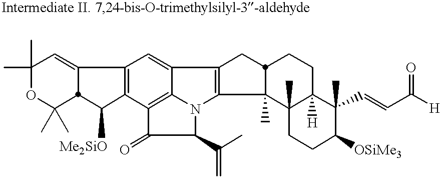

To Intermediate I (560 mg) in acetonitrile (10 mL) at 25° C. was added (Me₃Si)₂NH (1.8 mL) and the the solution was aged for 12 h. Additional (Me₃Si)₂NH (1.5 mL) and acetonitrile (3 mL) were then added. After 3 h, the solvent was removed under reduced pressure and the residue dried in vacuo for 1 h to yield pure title compound (870 mg, 100%) which required no purification. The product was characterized by proton NMR.

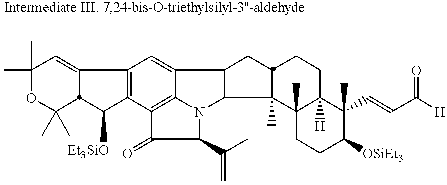

To Intermediate 1 (750 mg) in pyridine/DNF (30 rnL, 1/1) at room temperature was added Et₃SiOSO₂CF₃ (3.2 g) and aged for 20 min. The solution was diluted with ethyl acetate, washed with saturated CuSO₄(aq) (4 ×), water (1 ×), brine (1 ×), and dried (Na₂SO₄). The solution was filtered, concentrated under reduced pressure and pure product was obtained following flash chromatography on silica gel using 7/93 acetone/hexanes as eluant. The title compound thus obtained was characterized by ¹H NMR.

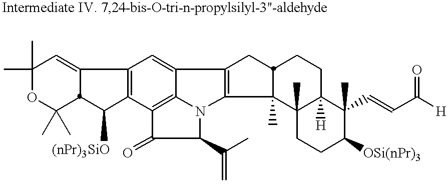

To Intermediate I (420 mg) and imidazole (540 mg) in CH₂Cl₂ (15 mL) at 0° C. was added (nPr)₃SiCl (1 mL) dropwise. After stirring for 30 min, the solution was warmed to room temperature for an additional 30 min and then quenched with ice-water. The organic phase was separated and washed with water, dried (NaSO₄), filtered and concentrated to give the pure product as a foam (620 mg). The product thus obtained was characterized by proton NMR.

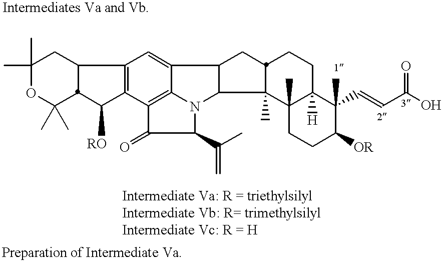

Method A

To Intermediate III (1 g) in tBuOH (25 mL) at 25° C. was added 2-methyl-2-butene (6 mL) and stirred for 5 min. A solution of NaOCl₂ (954 mg) and NaH₂PO₄.2H₂O (1.28 g) in water (10 mL) was then added. After 4 h, the solution was poured into saturated NH₄Cl(aq), extracted with CH₂Cl₂ (3×) and dried (Na₂SO₄). The solution was filtered and concentrated to dryness under reduced pressure. Pure title compound (725 mg) was obtained following flash chromatography on silica gel using gradient elution (5% to 25% EtOAc in hexanes).

Method B

A solution of KMnO₄ (1.3 g) in acetone (64 mL) and pH 7 phosphate buffer (21 mL) was prepared. To Intermediate III (3.63 g) in acetone (64 mL) was added the KMnO₄/buffer solution (˜20 mL) and the solution was aged for 30 min. Additional KMnO₄ solution (˜20 mL) was added every 30 min for 2 h. The solution was then cooled to 0° C. and 1M Na₂SO₃ was added until all of the KMnO₄ was reacted. The mixture was filtered and washed with 15/85 MeOH/acetone (2×). The filtrate was concentrated under reduced pressure to dryness and redissolved in water. The aqueous solution was extracted with 3/7 iPrOH/CHCl₃ (3×) and the organic layers were dried (Na₂SO₄). The solids were removed by filtration and the solution was evaporated to drynesss under reduced pressure. Pure title product (1.29 g) along with recovered starting aldehyde (˜1.3 g) was obtained following flash chromatography on silica gel using 2/8 EtOAc/hexanes as eluant.

Preparation of Intermediate Vb.

Following the procedure described for Intermediate Va and using Intermediate II, Intermediate Vb was prepared.

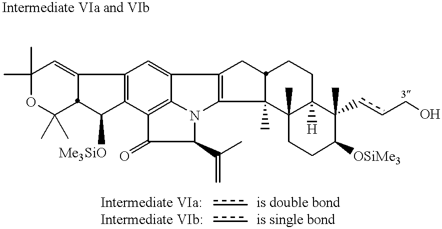

Method A

To Intermediate II (821 mg) in tetrahydrofuran (THF, 8 niL) at −78° C. was added L-Selectride® (Adrich, 1.07 mL, 1 M solution in THF) dropwise over 5 min. After 20 min, the solution was quenched by addition of saturated NH₄Cl(aq), extracted with CH₂Cl₂, washed with brine and dried (Na₂SO₄). The solution was filtered, concentrated under reduced pressure and purifed by flash chromatography on silica gel using 15/85 EtOAc/hexanes as eluant. The pure Intermediate VIa (571 mg) thus obtained was characterized by ¹H NMR.

Method B

To Intermediate II (1.0 g) in EtOAc (50 mL) at room temperature was added 10% Pd/C and a balloon atmosphere of hydrogen was established. After 5.5 h, the solution was filtered through Celite using EtOAc as eluant. The solution was concentrated under reduced pressure and purifed by MPLC chromatography on silica gel using 4/6 EtOAc/hexanes as eluant. The pure Intermediate VIa (726 mg, mobile product) and pure Intermediate VIb (70 mg, polar product) thus obtained were characterized by ¹H NMR.

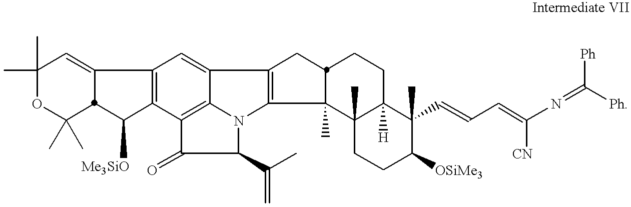

To (N-diphenylmethylene)amino acetonitrile (75 mg) in THF (0.5 mL) at −78° C. was added LiN(SiMe₃)₂ (340 μL, 1.0 M solution). The yellow solution was stirred at −78° C. for 5 min, placed in a 0° C. ice bath for 5 min and then recooled to −78° C. for 15 min. A solution of Intermediate II (65 mg in 0.8 niL THF) was added at −78° C. After 25 min, MeSO₂Cl (60 μL) was added. After 10 min, triethylamine (36 μL) was added and the reaction warmed first to 0° C. for 20 min and then room temperature of 2 h. The solution was rapidly filtered without workup through a 1 inch pad of silica gel using CH₂Cl₂ followed by 15/85 EtOAc/hexanes as eluant. The solution was concentrated to dryness under reduced pressure and used in the next step without any further manipulation or characterization.

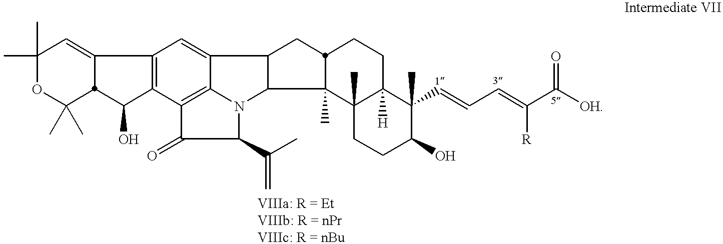

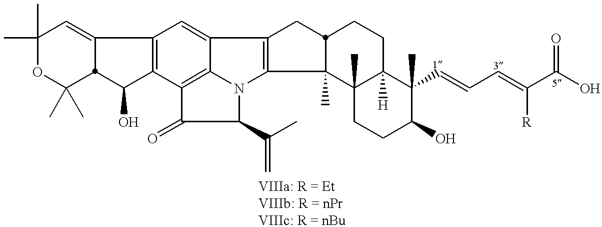

Step A. To Intermediate 1 (128 mg) in CH₂Cl₂ (10 mL) at 25° C. was added Ph₃P═C(Et)CO₂CH₂CH═CH₂ (320 mg). The solution was aged for 2 days and then additional Ph₃P═C(Et)CO₂CH₂CH═CH₂ (320 mg) was added. After one additional hour, the solution was purified without workup by flash chromatography on silica gel using 6/4 EtOAc/hexanes as eluant to yield pure allyl ester of Intermediate VIIIb (124 mg, 84%). The purified allyl ester of Intermediate VIIIa was characterized by proton NMR and mass spectrometry [m/z: 734.1. (M⁺+1)].

Step B. To the 5″ -allyl ester of Step A (160 mg) in 1/3 THF/CH₂Cl₂ (8 mL) at 25° C. was added (Ph₃P)₄Pd (13 mg) and morpholine (160 mg). The solution was aged for 6 h and the solution was purified without workup by flash chromatography on silica gel using 1/9 MeOH/CH₂Cl₂ as eluant to yield pure Intermediate VIIIa (112 mg, 74%). The purified Intermediate VIIIa was characterized by proton NMR and mass spectrometry [m/z: 693.4 (M⁺+1)].

Intermediates VIIIb and VIIIc were similarly prepared following the procedure for Intermediate VIIIa and using Ph₃P═C(n-Pr)CO₂CH₂CH═CH₂ and Ph₃P═C(n-Bu)CO₂CH₂CH═CH2, respectively.

General Procedures for the Preparation of Stabilized Wittig Reagents

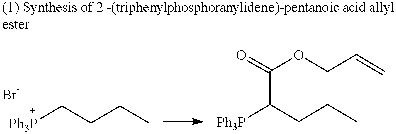

To a solution of Ph₃PCH₂CH₂CH₂CH₃.Br (20 g) in toluene (80 mL) at −78° C. was added nBuLi (31.3 mL, 1.6 M in hexane) over 10 min. The cooling bath was removed and the solution was allowed to warm to 25° C. After 1 h at 25° C., the deep red solution was heated to reflux and a solution of allyl chloroformate (3.01 g) in toluene (10 mL) was then added dropwise. A white solid precipitated immediately. After 5 min, the mixture was cooled to 25° C. and the white solid was collected by filtration and discarded. The filtrate was concentrated under reduced pressure to yield an off-white solid (7.89 g, 78%) that was dried in vacuuo and used with no further purification. The purified product was characterized by proton NMR.

(2) 1-Ethyl-3-triphenylphosphoranylidine-pyrrolidin-2-one (reference: J. Med. Chem. 1987, v30, pl995-1998).

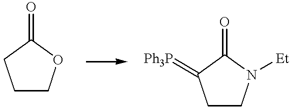

A mixture of γ-butyrolactone (100 g) and PBr₃ (2 mL) was heated at 110° C. while Br₂ (50 mL) was added slowly dropwise below the surface of the solution. The solution was cooled to 50° C. and DMF (0.1 ImL) was added. The solution was heated to 90° C. and SOCl₂ (100 mL) was added dropwise and the solution was aged for 3 h. The BrCH₂CH₂CH(Br)C(O)Cl thus prepared was used with no purification. Ethylamine (17 mL, 70 weight % in water) was diluted with water (50 mL) and CHCl₃ (30 mL) and cooled to 12° C. A portion of the acid chloride solution (20 g) was diluted with CHCl₃ (30 mL) and added dropwise to the EtNH₂ solution. After 30 min, the solution was poured into brine, extracted with CHCl₃ and dried (MgSO₄). The solution was filtered and concentrated to dryness and the residue (10.15 g) was used with no further purification. To the BrCH₂CH₂CH(Br)C(O)NHEt (10 g) thus obtained at 0° C. was added DMF (20 nL), benzene (60 mL) and treated with NaH (1.36 g, 60% dispersion in oil) in portions over 10 min. After 30 min, the solution was poured into water and extracted with EtOAc. The combined organic layers were washed with water, brine and dried (MgSO₄). The solution was filtered and concentrated to dryness to yield crude product (4.7 g) which was used with no further purification. To the 3-bromo-1-ethyl-pyrrolidinone (4.7 g) in THF (15 mL) was added Ph₃P (6.82 g) and the solution heated at reflux for 16 h. The volatiles were removed under reduced pressure and residue was dissolved in EtOH (30 niL) to which was added iPr₂NEt (15 mL). The solution was heated to reflux for 3 h and concentrated to dryness to yield the desired Wittig reagent which was used with no further purification. The Wittig reagent thus obtained was characterized by ¹H NMR.

(3) 1-Cyclopentyl-3-triphenylphosphoranylidine-succinamide (reference: Tetrahedron, 1968, 24, 2241).

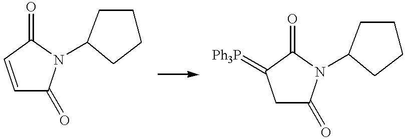

To Ph₃P (3.7 g) in glacial acetic acid (40 mL) was added 1-cyclopentyl-maleimide and the solution was heated to reflux for 1 h. The volatiles were removed under reduced pressure and the residue was dissolved in CH₂Cl₂ (100 mL). The CH₂Cl₂ solution was washed with saturated NaHCO₃(aq), brine and dried (MgSO₄). The solution was filtered, concentrated to dryness to yield the desired Wittig reagent which was used with no further purification. The Wittig reagent thus obtained was characterized by ¹H NMR.

(4) 3-triphenylphosphoranylidine-glutaramide (reference: Synthesis, 1998, 325).

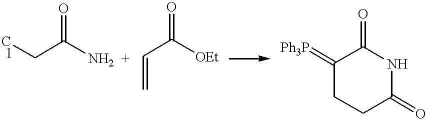

To alpha-chloroacetamide (3.8 g) in nitromethane (100 mL) was added Ph₃P (10.4 g) and the solution was refluxed for 30 h. The volatiles were removed under reduced pressure and MeOH (10 mL) was added followed by NaOMe (11.24 niL, 0.5 M solution in MeOH) and the solution was aged for 1 h. To this solution was then added ethyl acrylate (0.61 mL) and the solution was aged for 48 h. The volatiles were removed under reduced pressure and the residue was partioned between CH₂Cl₂ and water. The organic layer was separated, dried (MgSO₄), filtered and concentrated to dryness. The Wittig reagent (1.68 g) thus prepared was characterized by ¹H NMR and was used with no further purification.

The following examples are provided to illustrate the invention and are not to be construed as limiting the scope of the invention in any manner.

### EXAMPLE 1

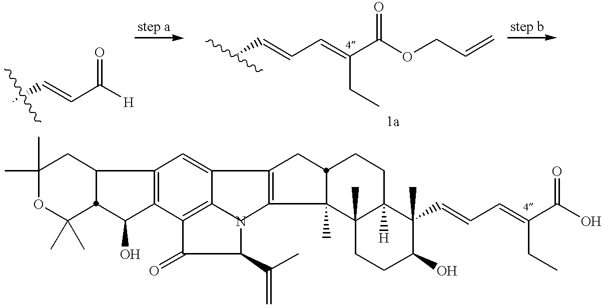

Step a:

To the 3″ -aldehyde (Intermediate I, 128 mg) in CH₂Cl₂ (10 mL) at 25° C. was added Ph₃P═C(Et)CO₂CH₂CH═CH₂ (320 mg). The solution was aged for 2 days and then additional Ph₃P═C(Et)CO₂CH₂CH═CH₂ (320 mg) was added. After one additional hour, the solution was purified without workup by flash chromatography on silica gel using 6/4 EtOAc/hexanes as eluant to yield pure 1a (124 mg, 84%). The purified product was characterized by proton NMR and mass spectrometry (m/z: 734.1 (M⁺+1)).

Step b:

To the product of step a (160 mg) in 1:3 THF/CH₂Cl₂ (8 mL) at 25° C. was added (Ph₃P)₄Pd (13 mg) and morpholine (160 mg). The solution was aged for 6 h and the solution was purified without workup by flash chromatography on silica gel using 1/9 MeOHICH₂Cl₂ as eluant to yield pure product (112 mg, 74%). The purified product was characterized by proton NMR and mass spectrometry (m/z: 693.4 (M⁺+1)).

### EXAMPLE 2

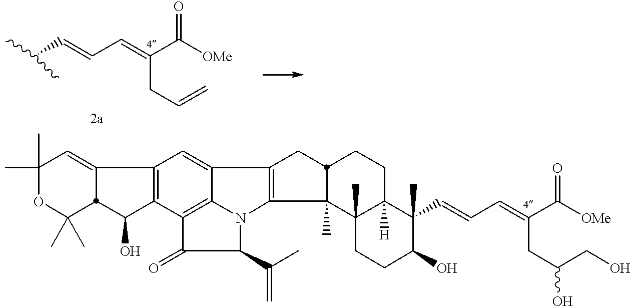

To a stirred solution of 2a (200 mg, prepared following the general procedure of Example 1, step a) in CH₂Cl₂ (10 mL) was added N-methylmorpholine-N-oxide (198 mg) followed by OSO₄ (0.34 mL, 4% solution in water). The solution was aged at 25° C. for 4 h and then poured into saturated Na₂S₂O₃(aq), extracted with CH₂Cl₂ and dried (Na₂SO₄). The solvents were removed in vacuo and pure product (182 mg, 86%) was obtained following flash chromatography on silica gel using 1/9 MeOH/CH₂Cl₂ as eluant. The purified product was characterized by proton NMR and mass spectrometry (m/z: 754.2 (M⁺+1)).

### EXAMPLE 3

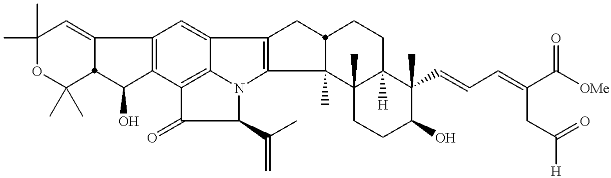

To a solution of the product of Example 2 (100 mg) in MeOH (6 mL) at 0° C. was added pyridine (11 μL) followed by Pb(OAc)₄ (60 mg) and the cooling bath was removed. After 10 min at 25° C., the solution was diluted with CH₂Cl₂ (30 mL) and poured into saturated Na₂S₂O₃(aq). The organic layer was washed with brine, dried (Na₂SO₄), filtered and concentrated under reduced pressure. Pure product (93 mg, 96%) was obtained following flash chromatography on silica gel using 1/9 MeOHI/CH₂Cl₂ as eluant. The purified product was characterized by proton NMR and mass spectrometry (m/z: 722.1 (M⁺+1)).

### EXAMPLE 4

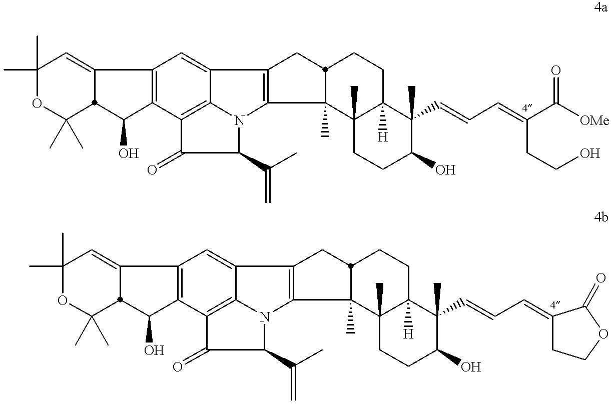

To the product of Example 3 (72 mg) in MeOH (3 mL) at 25° C. was added NaBH₃CN (8 mg). After 1 h, the solution was diluted with CH₂Cl₂, poured into saturated brine and dried (Na₂SO₄). The solution was filtered, concentrated under reduced pressure and pure products (60 mg 5a, 83%; 5 mg 5b) were obtained following preparative TLC on silica gel using 5/95 MeOH/CH₂Cl₂ as eluant. The purified product was characterized by proton NMR and mass spectrometry (m/z: 741.2 (M⁺+1) for 4a and m/z: 709.1 (M⁺+1) for 4b).

### EXAMPLE 5

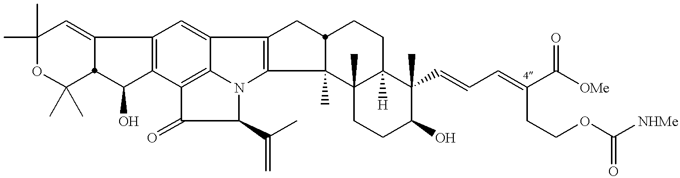

To compound 4a (18 mg) in CH₂Cl₂ (1 mL) at 0° C. was added (p-NO₂)PhOC(O)Cl (6 mg) followed by pyridine (3 μL). After 2 h, additional (p-NO₂)PhOC(O)Cl (6 mg) was added and the solution aged for 2 h. The reaction then was warmed to RT and methylamine (5 drops, 1 M solution in CH₂Cl₂) was added. After 1 h, the reaction was quenched by addition of saturated brine, extracted with CH₂Cl₂, dried (Na₂SO₄), filtered and concentrated under reduced pressure. Pure product was obtained following PTLC (1×1000 μm silica gel plate) using 6/4 EtOAc/hexanes as eluant. Pure product (3.3 mg) was characterized by ¹H NMR and mass spectrometry [m/z: 781.4 (M⁺+1)].

### EXAMPLE 6

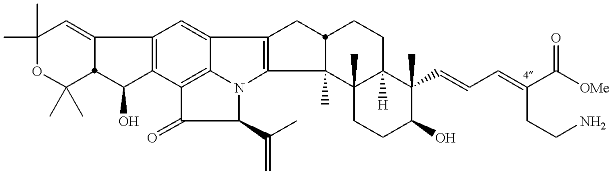

To the product of Example 3 (40 mg) in MeOH (5 mL) at 0° C. was added NH₄OAc (854 mg) and 3 Å molecular sieves (400 mg) followed by NaBH₃CN (4.2 mg) and the cooling bath was removed. After 30 min at 25° C., the solution was filtered and concentrated under reduced pressure. The residue was purified by preparative TLC on silica gel (4×1000 μm plates) using 1/9 MeOH/CH₂Cl₂ as eluant. The purified product (13 mg, 33%) was characterized by proton NMR and mass spectrometry (m/z: 723.5 (M⁺+1)).

### EXAMPLE 7

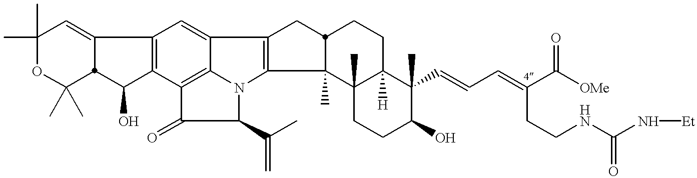

To the product of Example 6 (5 mg) in CH₂Cl₂ (1 mL) at RT was added ethyl isocyanate (10 μL). After 1 h, the reaction was quenched by addition of saturated brine, extracted with CH₂Cl₂, dried (Na₂SO₄), filtered and concentrated under reduced pressure. Pure product was obtained following PTLC (1×500 μm silica gel plate) using 6/4 EtOAc/hexanes as eluant. Pure product was characterized by ¹H NMR and mass spectrometry (m/z: 795.4 (M⁺+1)).

### EXAMPLE 8

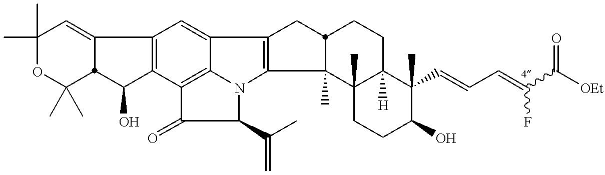

To (EtO)₂P(O)CH(F)CO₂Et (47 μL) in THF (1 mL) at −78° C. was added KN(SiMe₃)₂ (0.445 mL, 0.5 M in toluene) dropwise. After 15 min, the (EtO)₂P(O)CF(Li)CO₂Et solution (0.77 mL) was added dropwise over 5 min to 7,24-bis-O-trimethylsilyl-3″ -aldehyde (Intermediate II, 90 mg) in THF (1.8 mL) at 0° C. After 5 min, the solution was poured into saturated brine:saturated NaHCO₃ (˜1:1), extracted with CH₂Cl₂ and dried (Na₂SO₄). The solution was filtered and concentrated under reduced pressure. The residue was dissolved in 200° EtOH (1 mL) at 25° C. and pyridinium para-toluenesulfonate (23 mg) was added. After 10 min, the solvent was removed under a stream on nitrogen. Pure product (66 mg, 65%) as a mixture of E,E and E,Z isomers (˜1:1) was obtained following preparative TLC purification on silica gel (1×1500 μm plate) using 1/1 EtOAc/hexanes as eluant. The purified product was characterized by proton NMR.

### EXAMPLE 9

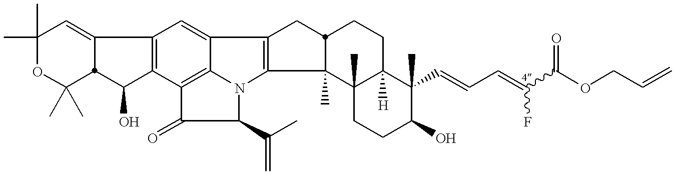

To the product of Example 9 (51 mg) was added allyl alcohol (2 mL) followed by Ti(OiPr)₄ (10 μL) and the solution was heated to 140° C. for 3 h. The solvent was removed under reduced pressure and the residue was dissolved in EtOAc and filtered through a 1.5 inch pad of silica gel using EtOAc as eluant and then concentrated. The product thus purified (52 mg, 100%) was characterized by proton NMR and mass spectrometry (m/z: 724.2 (M⁺+1)).

### EXAMPLE 10

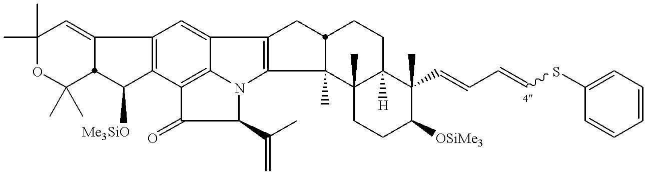

To Ph₃PCH₂SPh.Br (106 mg) in toluene (1 mL) at −78° C. was added KN(SiMe₃)₂ (0.5 mL, 0.5 M solution in toluene). The solution was then warmed to 0° C. and stirred for 30 min. To a solution of 7,24-bis-O-trimethylsilyl-3″-aldehyde (Intermediate II, 30 mg) in toluene (1 mL) at 0° C. was then added the Ph₃P═CHSPh solution (0.24 mL) dropwise. After 20 min at 0° C., additional Ph₃P═CHSPh solution (0.24 mL) was added. After 20 min, the solution was poured into saturated NaHCO₃, extracted with CH₂Cl₂ and dried (Na₂SO₄). The solution was filtered and concentrated under reduced pressure. Pure product (9 mg, 26%) was obtained following preparative TLC on silica gel (1×1500 μm plate) using 1/9 EtOAc/hexanes as eluant. The product thus purified was characterized by proton NMR.

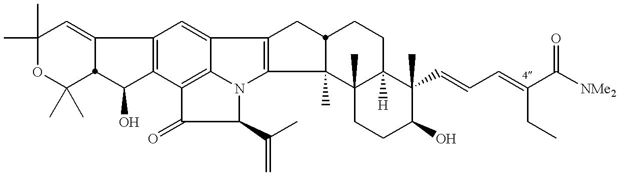

To the carboxylic acid product of Example 1 (30 mg) in CH₂Cl₂ (2 mL) at 25° C. was added N-hydroxybenzotriazole (5.8 mg) and diisopropylethylamine (8 μL) and the solution cooled to 0° C. To this solution was added BOP (22 mg) followed 15 min later by HNMe₂ (0.30 mL) and the cooling bath was removed. After 1 h at 25° C., the solution was poured into saturated NaHCO₃(aq), extracted with CH₂Cl₂ and dried (Na₂SO₄). The solution was filtered and concentrated under reduced pressure. Pure product (24 mg, 77%) was obtained following preparative TLC purification on silica gel (1×1000 μm plate) using 6/4 EtOAc/hexanes as eluant. The product thus purified was characterized by proton NMR and mass spectrometry (m/z: 721.5 (M⁺+1)).

### EXAMPLE 12

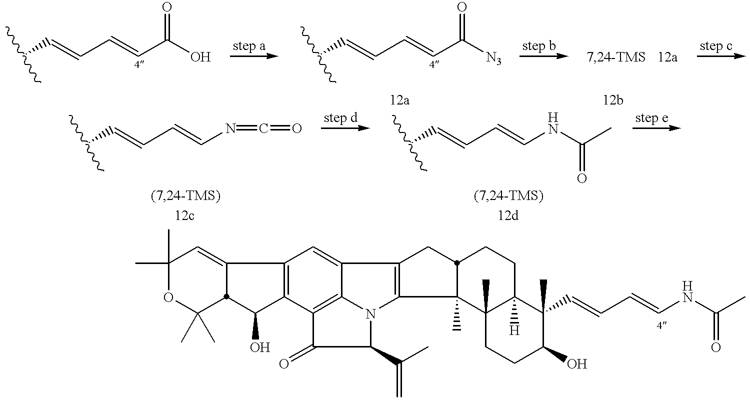

Step a:

To the 4″-carboxylic acid (90 mg, prepared following the general procedure of Example 1) in CH₂Cl₂ (6 mL) at 25° C. was added diisopropylethylamine (600 μL) followed by (PhO)₂P(O)N₃ (600 μL) and the solution was heated to reflux for 1 h. The solution was cooled to 25° C. and purified without workup by preparative TLC on silica gel using 4/6 EtOAc/hexanes as eluant to yield pure acyl azide 12a (68 mg, 71%). The product thus obtained was characterized by proton NMR.

Step b:

To the product of step a (68 mg) in MeCN (5 mL) was added HN(SiMe)₃ (0.1 mL) and aged for 1 h. The volatiles were removed under reduced pressure and pure product was obtained following preparative TLC on silica gel using 1/9 EtOAc/hexanes as eluant. The product thus obtained was characterized by proton NMR.

Step c:

To the product of step b (12 mg) was added PhMe (2 mL) and the solution was heated to 80° C. for 30 min. The solvent was removed under reduced pressure and the product obtained was characterized by proton NMR without workup or purification.

Step d:

To the product of step c (10 mg) in THF (0.4 mL) at −78° C. was added MeMgCl (15 μL, 3 M solution in THF). The solution was aged for 30 min and was then quenched by addition of saturated NH₄Cl(aq), extracted with CH₂Cl₂ and dried (Na₂SO₄). Pure product was obtained following preparative TLC on silica gel using 3/7 EtOAc/hexanes as eluant. The product thus obtained was characterized by proton NMR.

Step e:

To the product of step d (18 mg) at 25° C. in absolute ethanol (1 mL) was added pyridinium para-toluenesulfonate (11 mg). After 10 min, the solvent was removed under reduced pressure and pure product (10 mg, 69%) was obtained following preparative TLC on silica gel (1×1000 μm plate) using 4/6 acetone/hexanes as eluant. The product thus obtained was characterized by proton NMR and mass spectrometry (m/z: 679.4 (M⁺+1)).

### EXAMPLE 13

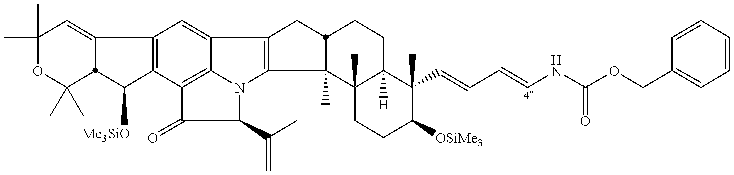

To the product of Example 12, step c (12c) in methylene chloride at RT was added benzyl alcohol and triethyl amine. The solution was aged for 2 h at RT and then the volatiles were removed under reduced pressure. Pure product was obtained following preparative TLC on silica gel using 2/8 EtOAc/hexanes as eluant. The product thus obtained was characterized by proton NMR.

### EXAMPLE 14

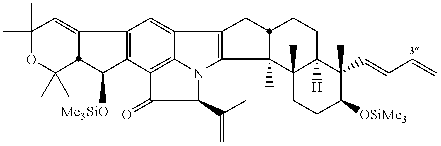

To the 7,24-bis-O-trimethylsilyl 3″ -aldehyde (Intermediate II, 25 mg) in THF (0.65 mL) at 25° C. was added (Cp)₂TiMe₂ (0.20 mL, 1M in toluene) and the solution was heated to 70° C. for 2 h. The golden dark-orange solution was cooled to 25° C. and purified without workup by centrifugal TLC (chromatatron) eluting first with hexanes, then 3/7 EtOAc/hexanes. The purified product (10 mg, 40%) was characterized by proton NMR.

### EXAMPLE 15

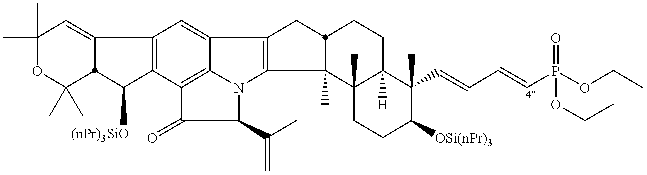

To a solution of CH₂[PO(OEt)2]2 (19 mg) in THF (0.5 nL) at −78° C. was added a (IM) solution of lithium hexamethyldisilazane (0.066 mL), and the mixture was stirred for 30 min. A solution of Intermediate IV (14 mg) in THF (0.5 mL) was then added to the anion of CH₂[PO(OC₂H₅)₂]₂ at −78° C. and the solution was aged for 2h. The reaction was quenched with 10% citric acid, and the crude product isolated after a work-up was purified by PTLC on silica gel (Analtech 1000 μm plates) using EtOAc/Hexane (2/1) as the eluant. Yield: 8 mg (55%). The product obtained was characterized by proton NMR and mass spectrometry [m/z: 758.3 (M⁺+1) and 682.8 (M-75)].

### EXAMPLE 16

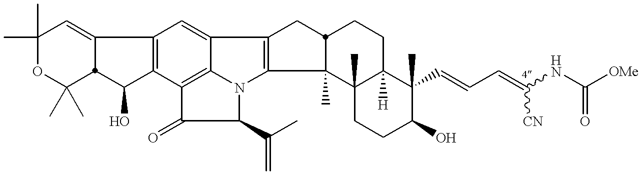

To Intermediate VII (25 mg) in MeOH (2 mL) at RT was added 2N HCl (50 μL) and the solution was aged for 30 min. The solution was aged for 30 min, cooled to 0° C. and neutralized with saturated NaHCO₃(aq). The solution was extracted with EtOAc, dried (Na₂SO₄), filtered and concentrated under reduced pressure. The crude 4″ -amine (18 mg) thus obtained was dissolved in CH₂Cl₂ (0.35 mL) at rt and dimethylpyrocarbonate (0.1 mL) was added. The solution was aged for 56 h. The volatiles were removed under reduced pressure and two pure, isomeric products were obtained following PTLC on silica gel (2×1000 μm plates) using 1/2 acetone/hexanes as eluant. The E and Z olefinic products were characterized by ¹H NMR and MS [m/z: 720.5 (M⁺+1) for each]. Yield: 5.4 mg mobile isomer A and 5.6 mg polar isomer B.

### EXAMPLE 17

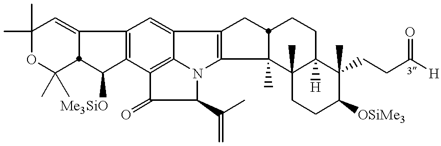

To Intermediate II (10 mg) in EtOAc (1 mL) at RT was added 10% Pd/C (1 mg) and an atmosphere of hydrogen was established using a balloon. After 4 h, the catalyst was removed by filtration through Celite using EtOAc as eluant and the solvent was removed under reduced pressure. Pure product (4 mg) was obtained following PTLC on silica gel (1×500 μm plate) using 2/8 acetone/hexanes as eluant. The product thus obtained was characterized by ¹H NMR.

### EXAMPLE 18

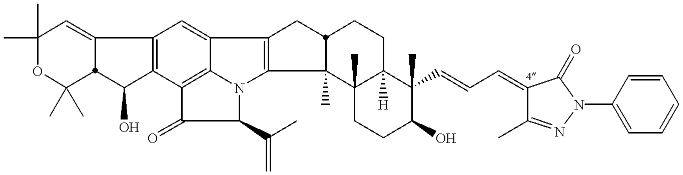

To Intermediate I (20 mg) in benzene (2 mL) at rt was added 3-methyl-1-phenyl-2-pyrazolin-5-one (40 mg). After 1 h, the solution was purified without workup by PTLC on silica gel (2×1000 μm plates) using 4/6 acetone/hexanes to yield pure product (19 mg). The product thus obtained was characterized by ¹H NMR and MS (m/z: 781.3 (M⁺+1)).

### EXAMPLE 19

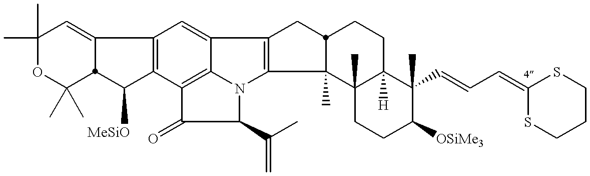

To 2-dimethylphosphonato-[1,3]dithiane (105 mg) in THF (0.75 mL) at −78° C. was added nBuLi (0.26 mL, 1.6M in hexanes). The solution was warmed to 0° C. over 30 min and then re-cooled to −78° C. To the ylide thus generated was added Intermediate I (100 mg) as a solution in THF (0.75 mL) and the solution was aged for 2 h at −78° C. Water was added to quench the reaction, the aqueous layer was extracted with CH₂Cl₂ and the combined organic layers were dried (Na₂SO₄), filtered and concentrated under reduced pressure. Pure dithiane product (82 mg) was obtained following PTLC on silica gel (2×1000 μm plates) using 1/9 EtOAc/hexanes as eluant. The product thus obtained was characterized by ¹H NMR.

Following the general descriptions of the previous examples and using appropriate 3″ -aldehyde precursors, the following compounds were prepared and deprotected as previously described. All compounds were characterized by mass spectrometry and/or ¹H NMR.

### EXAMPLE 20

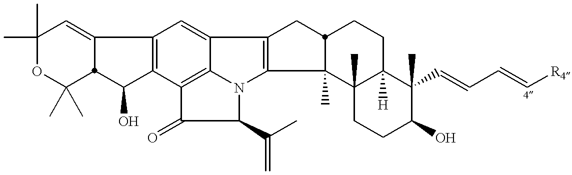

| Entry | R_{4″} Group*** | Mass Spec |
|---|---|---|
| 20a | H | |
| 20b | H(1′ = CH₂) | |
| 20c | CN | |
| 20d | CN (Z isomer) | |
| 20e | CN (2′-nat and 2′-epi) | |
| 20f | C(O)CH₃ | |
| 20g | CO₂H | 648.7(M⁺ + 1-H₂O) |
| 20h | CO₂CH₂CH═CH₂ | |
| 20i | CO₂CH₂Ph | |
| 20j | CO₂Et | |
| 20k | CO₂tBu | |
| 20l | CO₂tBu(1′-CHCO₂tBu) | |
| 20m | C(O)NH₂ | 665.3(M⁺ + 1) |
| 20n | C(O)(N-1-piperidinyl) | 733.5(M⁺ + 1) |
| 20o | C(O)(N-1-pyrrolidinyl) | 719.4(M⁺ + 1) |
| 20p | C(O)N(Me)CH₂CH₃ | 707.4(M⁺ + 1) |
| 20q | C(O)N(Me)CH₂Ph(4-Cl) | 803.4(M⁺ + 1) |
| 20r | C(O)NH₂ (Z isomer) | 665.1(M⁺ + 1) |
| 20s | C(O)NHCH₂CH₂F | 711.4(M⁺ + 1) |
| 20t | C(O)NHCH₂Ph | 755.4(M⁺ + 1) |
| 20u | C(O)NH—Et | |
| 20v | C(O)NH-tBu | 721.8(M⁺ + 1) |
| 20w | C(O)NMe₂ | 693.4(M⁺ + 1) |
| 20x | NHAc (Z isomer) | 679.4(M⁺ + 1) |
| 20y | NHC(O)Et | 693.4(M⁺ + 1) |
| 20z | NHC(O)Et (Z isomer) | 693.4(M⁺ + 1) |
| 20aa | NHC(O)nBu | 721.5(M⁺ + 1) |
| 20bb | NHC(O)nPr | 707.4(M⁺ + 1) |
| 20cc | NHC(O)nPr (Z isomer) | 707.4(M⁺ + 1) |
| 20dd | P(O)(OEt)₂ | 758.3(M⁺ + 1) |
| 20ee | SPh | 730.0(M⁺ + 1) |

***(E isomer unless indicated)

### EXAMPLE 21

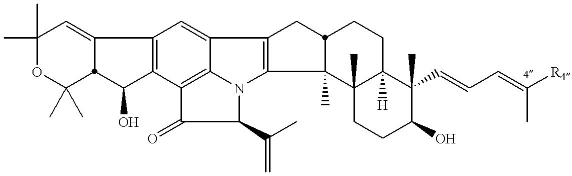

| Entry | R_{4″} Group*** | Mass Spec |
|---|---|---|
| 21a | 2-(4,5-dihydro-4-(R)-isopropyl-oxazolinyl) | 747.0(M⁺ + 1) |
| 21b | 2-(4,5-dihydro-4-(S)-C(O)NHEt-thiazolinyl) | 776.0(M⁺ + 1) |
| 21c | 2-(4,5-dihydro-4-(S)-C(O)NHMe- | |
| | thiazolinyl) | |
| 21d | 2-(4,5-dihydro-4-(S)-C(O)NHtBu- | 804.0(M⁺ + 1) |
| | thiazolinyl) | |
| 21e | 2-(4,5-dihydro-4-(S)-C(O)NHtBu- | |
| | thiazolinyl) (Z isomer) | |
| 21f | 2-(4,5-dihydro-4-(S)-C(O)NMe₂-thiazolinyl) | |
| 21g | 2-(4,5-dihydro-4-(S)-C(O)NMe₂-thiazolinyl) | 776.0(M⁺ + 1) |
| | (Z isomer) | |
| 21h | 2-(4,5-dihydro-4-(S)-isopropyl-oxazolinyl) | 747.0(M⁺ + 1) |
| 21i | 2-(4,5-dihydro-4-(spiro-cyclopentyl)- | 759.0(M⁺ + 1) |
| | oxazolinyl) | |
| 21j | 2-(4,5-dihydro-4-CO₂Me-thiazolinyl) | 779.0(M⁺ + 1) |
| 21k | 2-(4,5-dihydro-5-(R)-methyl-oxazolinyl) | 719.0(M⁺ + 1) |
| 21l | 2-(4,5-dihydro-5-(S)-methyl-oxazolinyl) | 719.0(M⁺ + 1) |
| 21m | 2-(4,5-dihydro-oxazolinyl) | 705(M⁺ + 1) |
| 21n | 2-furfuryl (E & Z) | |
| 21o | 2-thienyl | |
| 21p | 5-(1-Ph-tetrazolyl) | |
| 21q | 5-(1-Ph-tetrazolyl) (Z isomer) | |
| 21r | 5-(N-1-(2-cyanoethyl)-tetrazolyl) | |
| 21s | 5-(N-1-(2-cyanoethyl)-tetrazolyl) (Z isomer) | |
| 21t | 5-(N—Et-tetrazolyl) | |
| 21u | 5-[3-(Ph(3,5-CF₃))-isoxazolyl] | |
| 21v | 5-[3-(Ph(4-tBu))-isoxazolyl] | |
| 21w | 5-tetrazolyl | |
| 21x | CH═NNHSO₂Ph(4-Me) | |
| 21y | CH═NOCH₂Ph | |
| 21z | CH═NOMe | |
| 21aa | CH₂CO₂allyl | 734.6(M⁺ + 1) |
| 21bb | CH₂CO₂Me | 708.5(M⁺ + 1) |
| 21cc | CH₂CO₂nPr | 736.4(M⁺ + 1) |
| 21dd | CN | |
| 21ee | CN (Z isomer) | |
| 21ff | N-1-(5-Me-tetrazolyl) | 718.2(M⁺ + 1) |
| 21gg | NHC(O)Et | |
| 21hh | NHC(O)Me | 617.4(M − 75) |
| 21ii | NHC(O)Me(1″,2″-dihydro) | 635.5(M − 75) |
| 21jj | NHC(O)nBu | |
| 21kk | NHC(O)nPr | 645.6(M − 75) |
| 21ll | NHC(O)P(O)Ph₂ | 879.2(M⁺ + 1) |
| 21mm | NHC(O)Ph(2,4,6-Me) | 797.2(M⁺ + 1) |
| 21nn | NHC(O)Ph(4-Cl) | 713.4(M − 75) |
| 21oo | NHC(O)S(CH₂)₃Ph | |
| 21pp | NHC(O)SEt | |
| 21qq | OSO₂CF₃ | |
| 21rr | OSO₂CF₃ (2′-nat and 2′-epi) | |

***(E isomer unless indicated)

### EXAMPLE 22

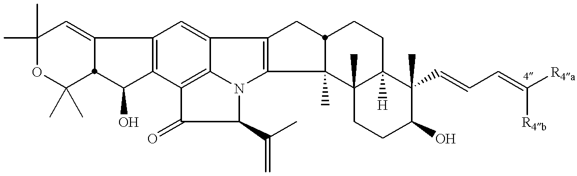

| Entry | R_{4″a} Group | R_{4″b} Group | Mass Spec |
|---|---|---|---|
| 22a | C(O)(N-1-piperidinyl) | Et | 761.5(M⁺ + 1) |
| 22b | C(O)(N-1-pyrrolidinyl) | Et | 747.5(M⁺ + 1) |
| 22c | C(O)CF₂Cl | CO₂Me | |
| 22d | C(O)Me | C(O)NEt₂ | |
| 22e | C(O)Me | CF₃ | |
| 22f | C(O)Me | NH—Me | |
| 22g | C(O)Me | NHPh(2-OMe) | |
| 22h | C(O)Me | NMe₂ | |
| 22i | C(O)N(Me)CH₂Ph(4-Cl) | Et | 831.4(M⁺ + 1) |
| 22j | C(O)N(Me)Et | CH₂OH | |
| 22k | C(O)NH₂ | CH₂CH═CH₂ | 705.3(M⁺ + 1) |
| 22l | C(O)NH₂ | Et | 693.4(M⁺ + 1) |
| 22m | C(O)NH₂ | nBu | 721.3(M⁺ + 1) |
| 22n | C(O)NH₂ | nPr | 707.4(M⁺ + 1) |
| 22o | C(O)NH₂ | OMe | 677.3(M⁺ + 1) |
| 22p | C(O)NH₂ | OMe | 695.4(M⁺ + 1) |
| 22q | C(O)NH₂ (2′-nat and 2′- | CH₂CH═CH₂ | 705.3(M⁺ + 1) |
| | epi) | | |
| 22r | C(O)NH₂ (2′-nat and 2′- | nPr | 707.4(M⁺ + 1) |
| | epi) | | |
| 22s | C(O)NHC(Me)₂C≡CH | CH₂C(O)NHC(Me)₂C≡CH | |
| 22t | C(O)NHCH₂CF₃ | CH₂CH═CH₂ | 787.4(M⁺ + 1) |
| 22u | C(O)NHCH₂CF₃ | Et | 775.4(M⁺ + 1) |
| 22v | C(O)NHCH₂CH₂F | CH₂CH(OH)CH₂OH | 785.4(M⁺ + 1) |
| 22w | C(O)NHCH₂CH₂F | CH₂CH═CH₂ | 751.4(M⁺ + 1) |
| 22x | C(O)NHCH₂Ph(4-F) | CH₂CH(OH)CH₂OH | 847.5(M⁺ + 1) |
| 22y | C(O)NHCH₂Ph(4-F) | CH₂CH═CH₂ | 813.5(M⁺ + 1) |
| 22z | C(O)NH—Et | CH₂CH(OH)CH₂OH | 767.4(M⁺ + 1) |
| 22aa | C(O)NH—Et | CH₂CH═CH₂ | 733.4(M⁺ + 1) |
| 22bb | C(O)NH—Et | Et | 721.4(M⁺ + 1) |
| 22cc | C(O)NH—Et | nBu | 749.4(M⁺ + 1) |
| 22dd | C(O)NH—Et | nPr | 735.5(M⁺ + 1) |
| 22ee | C(O)NH—Et | OMe | 705.4(M⁺ + 1) |
| 22ff | C(O)NH—Et (2′-nat and | CH₂CH═CH₂ | 733.3(M⁺ + 1) |
| | 2′-epi) | | |
| 22gg | C(O)NH—Et (2′-nat and | F | 711.1(M⁺ + 1) |
| | 2′-epi) | | |
| 22hh | C(O)NH—Et (2′-nat and | nPr | 735.5(M⁺ + 1) |
| | 2′-epi) | | |
| 22ii | C(O)NHiBu | CH₂C(O)NH-iBu | |
| 22jj | C(O)NH-iPr | CH₂CH(OH)CH₂OH | 781.4(M⁺ + 1) |
| 22kk | C(O)NH-iPr | CH₂CH═CH₂ | 747.3(M⁺ + 1) |
| 22ll | C(O)NHMe | CH₂CH(OH)CH₂OH | 753.4(M⁺ + 1) |
| 22mm | C(O)NH—Me | CH₂CH═CH₂ | 719.4(M⁺ + 1) |
| 22nn | C(O)NH—Me | CH₂CH₂-epoxide | |
| 22oo | C(O)NH—Me | Et | |
| Z2pp | C(O)NH—Me (2′-nat and | Et | 707.4(M⁺ + 1) |
| | 2′-epi) | | |
| 22qq | C(O)NHMe (E & Z) | C(O)Me | |
| 22rr | C(O)NHPh(2-OMe) | C(O)Me | |
| | (E & Z) | | |
| 22ss | C(O)NH-tBu | CH₂CH(OH)CH₂OH | 795.3(M⁺ + 1) |
| 22tt | C(O)NH-tBu | CH₂CH═CH₂ | 761.3(M⁺ + 1) |
| 22uu | C(O)NH-tBu | Et | 749.5(M⁺ + 1) |
| 22vv | C(O)NH-tBu | F | 739.0(M⁺ + 1) |
| 22ww | C(O)NH-tBu | nBu | 777.4(M⁺ + 1) |
| 22xx | C(O)NH-tBu | nPr | 764.4(M⁺ + 1) |
| 22yy | C(O)NH-tBu | OMe | |
| 22zz | C(O)NH-tBu (2′-nat and | CH₂CH═CH₂ | |
| | 2′-epi) | | |
| 22aaa | C(O)NH-tBu (2′-nat and | nPr | 763.5(M⁺ + 1) |
| | 2′-epi) | | |
| 22bbb | C(O)NMe₂ | CH₂CH(OH)CH₂OH | 767.5(M⁺ + 1) |
| 22ccc | C(O)NMe₂ | CH₂CH═CH₂ | 733.4(M⁺ + 1) |
| 22ddd | C(O)NMe₂ | CH₂CH₃ | 721.5(M⁺ + 1) |
| 22eee | C(O)NMe₂ (E & Z) | C(O)Me | |
| 22fff | C(O)Ph | OMe | 726.1(M⁺ + 1) |
| 22ggg | C(O)Ph (Z isomer) | C(O)Me | |
| 22hhh | C(O)Ph(4-F) (E & Z) | CO₂Me | |
| 22iii | CF₃ | C(O)Me | |
| 22jjj | CF₃ (23,24-dehydro) | C(O)Me | |
| 22kkk | CH₂CH₂NHC(O)NH—Et | CO₂Me | 795.4(M⁺ + 1) |
| 22lll | CH₂CH₂OC(O)NH-iPr | CO₂Me | 809.4(M⁺ + 1) |
| 22mmm | CH₂CH₂OC(O)NH-tBu | CO₂Me | 823.4(M⁺ + 1) |
| 22nnn | CN | CN | |
| 22ooo | CN | CO₂CH₂CH═CH₂ | |
| 22ppp | CO₂CH₂CH═CH₂ | F | |
| 22qqq | CO₂H | Et | 693.4(M⁺ + 1) |
| 22rrr | CO₂H | F | |
| 22sss | CO₂H | nBu | 722.3(M⁺ + 1) |
| 22ttt | CO₂H | nPr | 708.4(M⁺ + 1) |
| 22uuu | CO₂H | OMe | 696.1(M⁺ + 1) |
| 22vvv | CO₂H (2′-nat and 2′-epi) | CH₂CH═CH₂ | 706.2(M⁺ + 1) |
| 22www | CO₂Me | 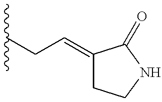 | 789.1(M⁺ + 1) |
| 22xxx | CO₂Me | 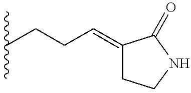 | 803.1(M⁺ + 1) |
| 22yyy | CO₂Me | 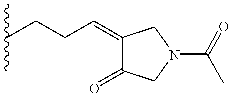 | 861.9(M + NH₄) |
| 22zzz | CO₂Me | 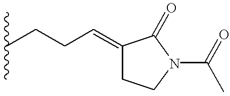 | 847.9(M + NH₄) |
| 22aaaa | CO₂Me | CH₂CH(OH)CH₂OH | 754.2(M⁺ + 1) |
| 22bbbb | CO₂Me | CH₂CH═CH₂ | 702.1(M − H₂0) |
| 22cccc | CO₂Me | CH₂CH₂CH(OH)CH₂OH | 769.1(M⁺ + 1) |
| 22dddd | CO₂Me | CH₂CH₂CH═CH₂ | 751.4(M + NH₄) |
| 22eeee | CO₂Me | CH₂CH₂NH₂ | 723.5(M⁺ + 1) |
| 22ffff | CO₂Me | CH₂CH₂NHC(O)NH—Et | 795.4(M⁺ + 1) |
| 22gggg | CO₂Me | CH₂CH₂OC(O)NH—Et | 795.5(M⁺ + 1) |
| 22hhhh | CO₂Me | CH₂CH₂OC(O)NH-iPr | 809.4(M⁺ + 1) |
| 22iiii | CO₂Me | CH₂CH₂OC(O)NH—Me | 781.4(M⁺ + 1) |
| 22jjjj | CO₂Me | CH₂CH₂OC(O)NH-tBu | 823.4(M⁺ + 1) |
| 22kkkk | CO₂Me | CH₂CH₂OH | 741.2(M + NH₄) |
| 22llll | CO₂Me | CH₂CHO | 722.1(M⁺ + 1) |
| 22mmmm | CO₂Me | CN | |
| 22nnnn | CO₂Me | NHCO₂CH₂Ph | 845.9(M + NH₄) |
| 22oooo | CO₂Me | Ph(4-F) | |
| 22pppp | CO₂Me (2′-nat and 2′-epi) | CH₂CH₂CH═CH₂ | — |
| 22qqqq | CO₂Me (2′-nat and 2′-epi) | CH₂CH₂CHO | 736.1(M⁺ + 1) |
| 22rrrr | CO₂Me (2′-nat and 2′-epi) | CH₂CH₂NH—Et | 751.6(M⁺ + 1) |
| 22ssss | CO₂Me (2′-nat and 2′-epi) | CN | |
| 22tttt | CO₂Me (2′-nat and 2′-epi) | NHCO₂CH₂Ph | 846.0(M + NH₄) |
| 22uuuu | F | C(O)NH—Et | 711.0(M⁺ + 1) |
| 22vvvv | F (2′-nat and 2′-epi) | C(O)NH—Et | 711.0(M⁺ + 1) |
| 22wwww | F (2′-nat and 2′-epi) | C(O)NH-tBu | 739.2(M⁺ + 1) |
| 22xxxx | NHC(O)Et | Et | 721.5(M⁺ + 1) |
| 22yyyy | NHC(O)Me | Et | 707.4(M⁺ + 1) |
| 22zzzz | NHC(O)Me | Et | 709.5(M⁺ + 1) |
| 22aaaaa | NHC(O)Me (E & Z) | CN | |
| 22bbbbb | NHC(O)nBu | H | 721.5(M⁺ + 1) |
| 22ccccc | NHC(O)NH-tBu | nBu | 778.1(M⁺ + 1) |
| 22ddddd | NHC(O)N-morpholinyl | nBu | 806.2(M⁺ + 1) |
| 22eeeee | NHC(O)OMe | CN | |
| 22fffff | NHC(O)OMe (Z isomer) | CN | |
| 22ggggg | NHCO₂CH₂Ph | CO₂Me | |

***(E isomer unless indicated)

### EXAMPLE 23

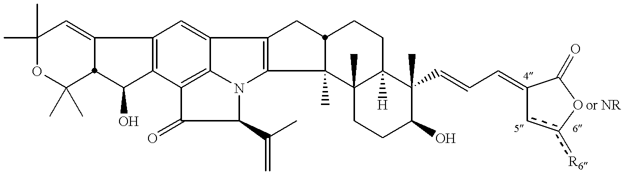

| Entry | R_{6″} | O or NR | 5″ - 6″ | Mass Spec |
|---|---|---|---|---|
| 23a | H | N-(2-pyridyl) | single | 768.4(M⁺ + 1) |
| 23b | H | N-allyl | single | 731.4(M⁺ + 1) |
| 23c | oxo | N-allyl | single | 745.4(M⁺ + 1) |
| 23d | H | N-cC₅H₉ | single | 759.5(M⁺ + 1) |
| 23e | oxo | N-cC₅H₉ | single | 755.1(M − H₂0) |
| 23f | oxo | N-cC₆H₁₁ | single | 769.0(M − H₂0) |
| 23g | oxo | N—CH₂CH₂F | single | 751.6(M⁺ + 1) |
| 23h | oxo | N—CH₂Ph | single | 777.1(M − H₂0) |
| 23i | H | N-cPr | single | 731.4(M⁺ + 1) |
| 23j | oxo | N-cPr | single | 745.2(M⁺ + 1) |
| 23k | oxo | NEt | single | 733.3(M⁺ + 1) |
| 23l | oxo | N-(1-pyrrolidinyl) | single | 774.6(M⁺ + 1) |
| 23m | oxo | N-(4-morpholinyl) | single | 790.4(M⁺ + 1) |
| 23n | oxo | N-[3-(5-Me- | single | 785.4(M⁺ + 1) |
| | | isoxazolyl)] | | |
| 23o | oxo | N-nBu | single | 761.4(M⁺ + 1) |
| 23p | H | NC(O)Me | single | 691.4(M⁺ + 1) |
| 23q | oxo | NCH₂CH₂CN | single | 757.4(M⁺ + 1) |
| 23r | H | NCH₂CH₂F | single | 737.4(M⁺ + 1) |
| 23s | OH | NCH₂CH₂F | single | 753.4(M⁺ + 1) |
| 23t | OMe | NCH₂CH₂F | single | 767.4(M⁺ + 1) |
| 23u | H | NCH₂CH₂OH | single | 735.4(M⁺ + 1) |
| 23v | OH | NCH₂Ph(4-F) | single | 815.4(M⁺ + 1) |
| 23w | H | N—Et | double | 717.4(M⁺ + 1) |
| 23x | H | N—Et | single | 719.5(M⁺ + 1) |
| 23y | OH | N—Et | single | 735.4(M⁺ + 1) |
| 23z | H | NH | single | 691.4(M⁺ + 1) |
| 23aa | oxo | NH | single | 705.3(M⁺ + 1) |
| 23bb | H | NH (2′-epi) | single | |
| 23cc | H | N-iBu | single | 747.5(M⁺ + 1) |
| 23dd | H | N-iPr | single | 733.5(M⁺ + 1) |
| 23ee | OH | N-iPr | single | 749.4(M⁺ + 1) |
| 23ff | oxo | NMe | single | 701.1(M − H₂0) |
| 23gg | H | N—Me | single | 705.4(M⁺ + 1) |
| 23hh | OH | N—Me | single | 721.4(M⁺ + 1) |
| 23ii | H | N-tBu | double | 745.5(M⁺ + 1) |
| 23jj | H | N-tBu | single | 747.5(M⁺ + 1) |
| 23kk | OMe | N-tBu | single | 777.4(M⁺ + 1) |
| 23ll | H | O | single | |
| 23mm | oxo | N—Ph | single | 763.1(M − H₂O) |
| 23nn | oxo | N—Ph(2-F) | single | 799.4(M⁺ + 1) |
| 23oo | oxo | N—Ph(3,5-diMe) | single | 810.6(M⁺ + 1) |
| 23pp | oxo | N—Ph(3-F) | single | 799.4(M⁺ + 1) |
| 23qq | oxo | N—Ph(3-Me) | single | 795.5(M⁺ + 1) |
| 23ff | oxo | N—Ph(4-Ac) | single | 823.6(M⁺ + 1) |
| 23ss | oxo | N—Ph(4-Cl) | single | 815.4(M⁺ + 1) |
| 23tt | oxo | N—Ph(4-Et) | single | 809.6(M⁺ + 1) |
| 23uu | oxo | N—Ph(4-F) | single | 799.4(M⁺ + 1) |
| 23vv | oxo | N—Ph(4-Me) | single | 795.4(M⁺ + 1) |
| 23ww | oxo | N—Ph(4-OMe) | single | 811.6(M⁺ + 1) |
| 23xx | oxo | N-tBu | single | 744.0(M − H₂O) |

### EXAMPLE 24

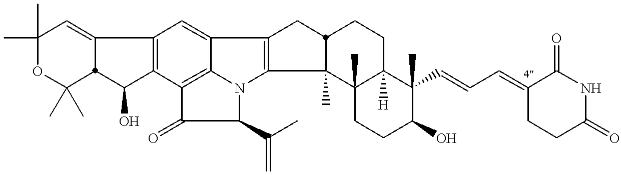

Following the general descriptions of Example 1 using the Intermediate I and 3-triphenylphosphoranylidine-glutaramide, the title compound was prepared as previously described and characterized by ¹H NMR and mass spectrometry (m/z: 719.3 (M⁺+1))

### EXAMPLE 25

Following the general descriptions of previous examples and using the appropriate 3″ -aldehyde precursor, the following compounds were prepared and deprotected as previously described. All compounds were characterized by mass spectrometry and/or ¹H NMR.

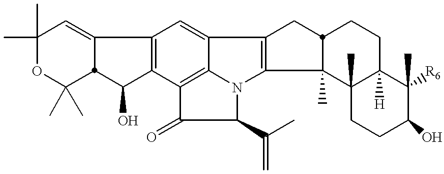

| Entry | R₆ | Mass Spec |
|---|---|---|
| 25a | 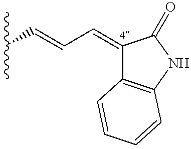 | 739.3(M⁺ + 1) |
| 25b | 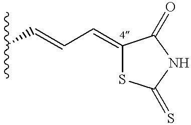 | 739.4(M⁺ + 1) |
| 25c | 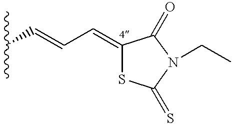 | 767.3(M⁺ + 1) |
| 25d | 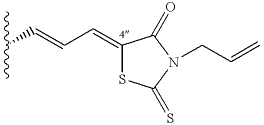 | 779.3(M⁺ + 1) |
| 25e | 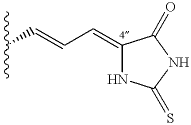 | 722.3(M⁺ + 1) |
| 25f | 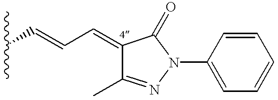 | 781.3(M⁺ + 1) |
| 25g | 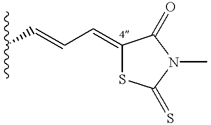 | 735.3(M⁺ + 1) |
| 25h | 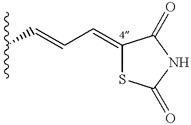 | 705.2 (M − H₂O) |
| 25i | 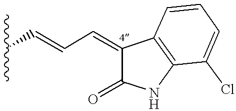 | 774.2(M⁺ + 1) |
| 25j | 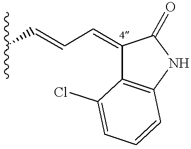 | |
| 25k | 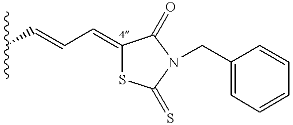 | 830.2(M⁺ + 1) |
| 25l | 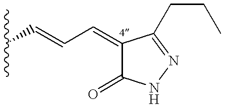 | 732.3(M⁺ + 1) |
| 25m | 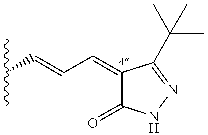 | 746.3(M⁺ + 1) |
| 25n | 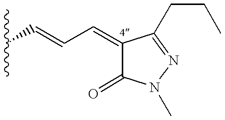 | 746.3(M⁺ + 1) |
| 25o | 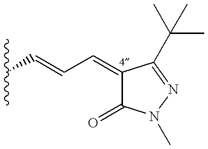 | 760.3(M⁺ + 1) |
| 25p | 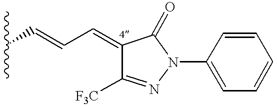 | 816.0 (M − H₂O) |
| 25q | 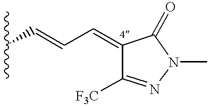 | 772.2(M⁺ + 1) |
| 25r | 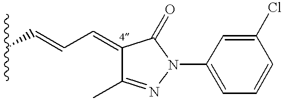 | 815.3(M⁺ + 1) |
| 25s | 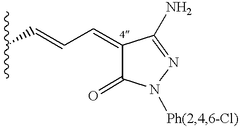 | |
| 25t | 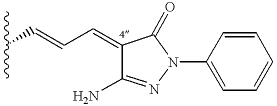 | 781.3(M⁺ + 1) |
| 25u | 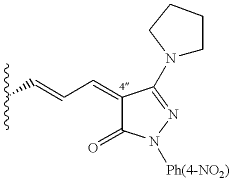 | 880.3(M⁺ + 1) |
| 25v | 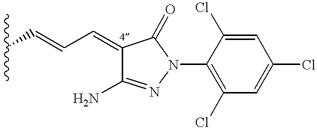 | 885.1(M⁺ + 1) |
| 25w | 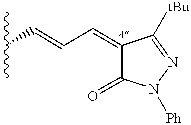 | 822.4(M⁺ + 1) |
| 25x | 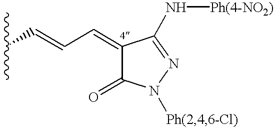 | 1006.2 (M⁺ + 1) |
| 25y | 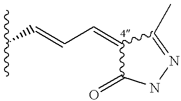 | 704.3(M⁺ + 1) |
| | 1:2 mixture of E and Z isomers | |
| 25z | 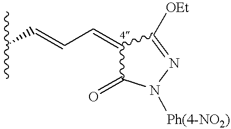 | 855.3(M⁺ + 1) |
| | 1 1 mixture of E and Z isomers | |
| 25aa | 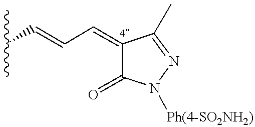 | 859.3(M⁺ + 1) |
| 25bb | 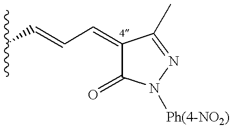 | 825.3(M⁺ + 1) |

### EXAMPLE 26

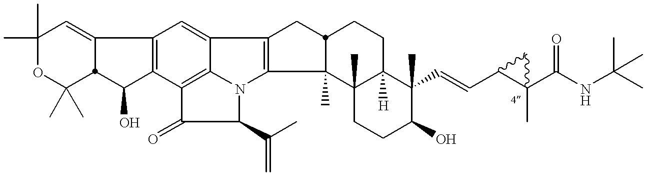

To a solution of N-(1,1-dimethylethyl)-nodulisporamide (50 mg) and Pd(OAc)₂ (3 mg) in CH₂Cl₂ (2 mL), a solution of CH₂N₂ in ether (generated from N-nitrosomethylurea) (0.5 mL) was added dropwise at room temperature. The resulting dark colored (N₂ evolution was observed) mixture was stirred for 16 h at room temperature and then filtered through a celite pad. The filtrate was concentrated in vacuo, and the residue was purified by PTLC using EtOAc/hexane (1/1) (three developments) to give the desired cyclopropane compounds: fast moving Isomer A (8 mg) and slower moving Isomer B (23 mg).

### EXAMPLE 27

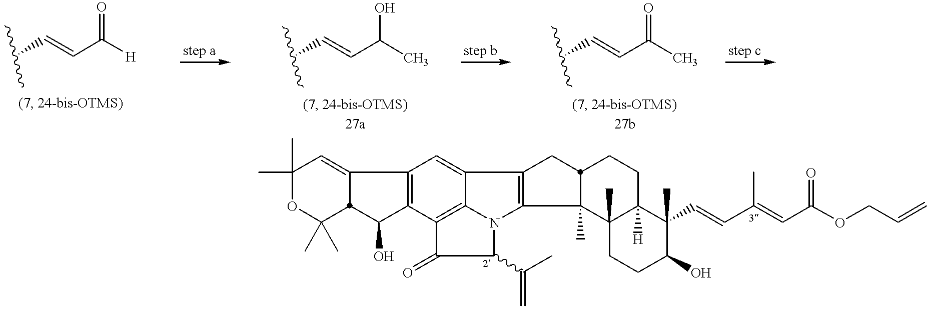

Step a:

To a stirred solution of Intermediate II (3.24 g) in diethyl ether (100 mL) at −78° C. was added MeMgBr (2.78 mL, 3.0 M solution in diethyl ether) over 10 min. After 30 min at −78° C., the mixture was quenched with methanol (1 mL), washed once with saturated NH₄Cl(aq) and the aqueous layer was extracted with diethyl ether (2×). The combined organic layers were washed with saturated NaHCO₃, brine and dried (Na₂SO₄). The solution was filtered and concentrated under reduced pressure. The intermediate 27a thus obtained was used with no further purification.

Step b:

The 3″ -alcohol 27a (3.05 g) was placed in methylene chloride (50 mL) at RT to which was added MnO₂ (5 g) and the solution was aged with vigorous stirring for 3 d. The solution was filtered and the filtrate evaporated under reduced pressure. Pure 27b (2.6 g) was obtained following flash chromatography on silica gel using 1/9 EtOAc/hexanes as eluant. The product was characterized by proton NMR.

Step c

To a stirred solution of (EtO)₂P(O)CH₂CO₂CH₂CH═CH2 (0.5 g) in THF/DMF (2 mL, 1/1) was added NaH (43 mg, 60% dispersion in oil) and the solution was aged for 1 h. The 3″ -methyl ketone 27b (165 mg) was added to the NaH/phosphonate solution and the mixture was heated to 60° C. for 4 h. The solution was cooled to RT and diluted with methylene chloride, washed with water (3×), brine (1×) and dried (Na2SO4). The solution was filtered and concentrated to dryness under reduced pressure. The residue was filtered through a 1.5″ thick pad of silica gel to remove baseline contaminants using 1/9 EtOAc/hexanes as eluant. The bis-protected product thus obtained was placed in ethanol (5 mL) at RT to which was added PPTS (2 mg). After 1 h, the solution was concentrated to dryness and was purified by flash chromatography to yield a mixture of 2′-epi (fast-moving product) and 2′-nat (slow moving product) stereoisomers. The products thus obtained were characterized by proton NMR.

### EXAMPLE 28

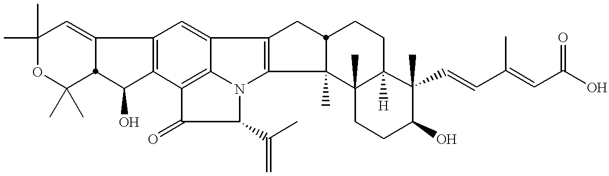

The 2′-epi allyl ester product (60 mg) of Example 27 was placed in methylene chloride (6 mL) to which was adde PdCl₂(PPh₃)2 (5 mg) followed by nBu₃SnH (0.15 mL). After 30 min, the solution was purified by flash chromatography on silica gel without workup to yield pure carboxylic acid derivative (35 mg) which was characterized by proton NMR and MS (m/z: 680.3 (M⁺+1-H₂O).

### EXAMPLE 29

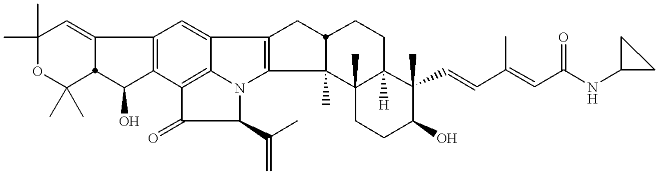

The 2′-nat-carboxylic product (8 mg) of Example 27 was placed in methylene chloride (2 mL) at RT to which was added HOBT (1.9 mg), (iPr)₂NEt (20.5 mg) and cyclopropyl amine (3.4 mg) followed by the addition of BOP reagent (15.6 mg). After 10 min, the solution was diluted with methylene chloride, washed with brine and dried (Na₂SO₄). The solution was filtered and concentrated under reduced pressure. Pure amide product (5.3 mg) was obtained following PTLC (1×1000 μm plate) on silica gel using 1/1 acetone/hexanes as eluant. The product thus obtained was characterized by proton NMR and MS (m/z: 719.3 (M⁺+1).

### EXAMPLE 30

Following the general procedure of Example 29, the following compounds were prepared and were characterized by mass spectrometry and/or proton NMR.

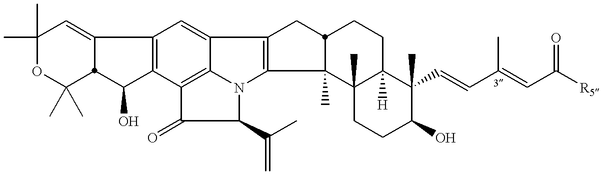

| Entry | R_{5″} Group | Mass Spec |
|---|---|---|
| 30a | NH-(N-1-piperizinyl) | |
| 30b | NHCH₂CH₂F | 725.3(M⁺ + 1) |
| 30c | NH—Et | 707.3(M⁺ + 1) |
| 30d | NH-iPr | 721.4(M⁺ + 1) |
| 30e | NH-iPr (2′-epi) | 721.4(M⁺ + 1) |
| 30f | NH—Me | 693.3(M⁺ + 1) |
| 30g | NH—Me (2′-epi) | 693.3(M⁺ + 1) |
| 30h | NHtBu | 735.4(M⁺ + 1) |
| 30i | NH-tBu (2′-epi) | 735.5(M⁺ + 1) |
| 30j | OH | 662.3(M⁺ + 1-H₂O) |

### EXAMPLE 31

The following derivatives were obtained by osmium-catalyzed dihydroxylation using the appropriate unsaturated precursor and following the general procedure used for the preparation of Intermediate I (method b). The cis-diol products thus obtained, which could be optionally separated (both R,R and S,S diastereomers were produced), were characterized by proton NMR.

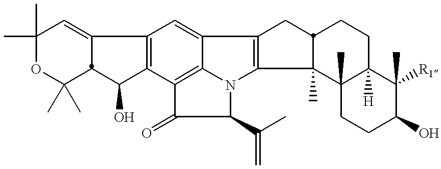

| Entry | R_{1″} Group |
|---|---|
| 31a | 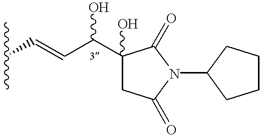 |
| 31b | 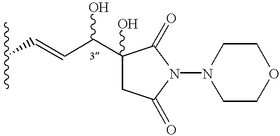 |
| 31c | 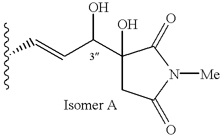 |
| 31d | 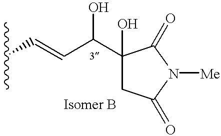 |
| 31e | 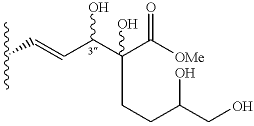 |

### EXAMPLE 32

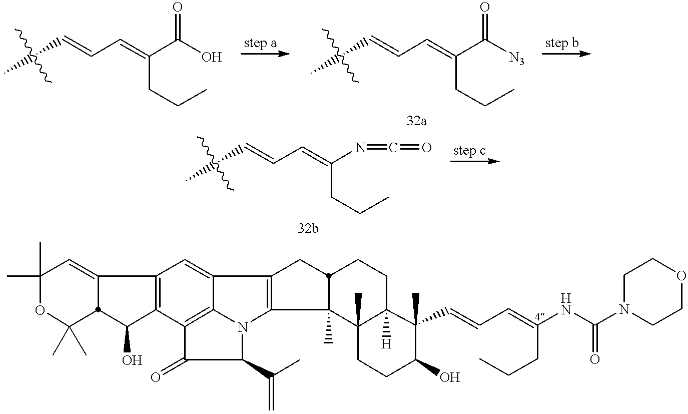

Step a:

To the 4″ -n-propyl substituted carboxylic acid (25 mg, prepared following the general procedures used for the preparation of the product of Example 2) in CH₂Cl₂ (2 mL) at 25° C. was added diisopropylethylamine (62 μL) followed by (PhO)₂P(O)N₃ (61 μL) and the solution was heated to reflux for 1 h. Pure acyl azide product 32a (21 mg, 81%) was obtained without workup following preparative TLC purification on silica gel (2×1000 μm plates) using 6/4 EtOAc/hexanes as eluant. The product thus purified was characterized by proton NMR.

Step b:

To the acyl azide of step a (21 mg) was added PhMe (2 mL) and the solution was heated to reflux for 1.5 h. The solution was cooled to 25° C. and concentrated to dryness to yield desired isocyanate 32b (18.6 mg) which was used with no further purification. The product thus obtained was characterized by proton NMR.

Step c:

To the isocyanate product of step b (10 mg) in methylene chloride (1 mL) was added morpholine (0.1 mL) and the solution was aged for 10 min. Pure urea product (7.9 mg) was obtained without workup following preparative TLC on silica gel (1×1000 μm plate) using 6/4 EtOAc/hexanes as eluant. The product thus obtained was characterized by proton NMR and mass spectrometry (m/z: 806.2 (M⁺+1)).

### EXAMPLE 33

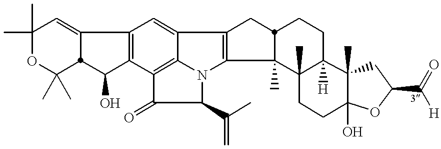

Nodulisporic acid A1 (Compound B) was converted into the corresponding 3″ -aldehyde following the general procedure as described for the preparation of Intermediate I. The product thus obtained was characterized by ¹H NMR and MS [m/z: 654.2 (M⁺+1)].

### EXAMPLE 34

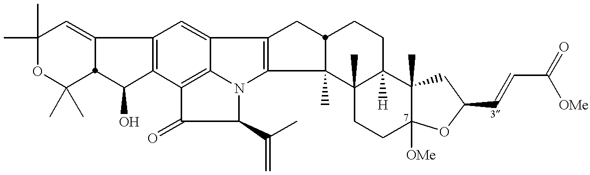

The product of Example 33 was converted into the corresponding enoate following the general conditions described for the preparation of the product of Example 1. During purification, transferring and concentration of the reaction product using methanol led to incorporation of a methoxyl at C7. The title product thus obtained was characterized by ¹H NMR and MS [m/z: 710.3 (M⁺+1)].

### EXAMPLE 35

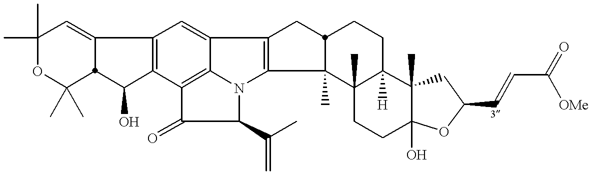

To the product of Example 34 (26 mg) in a mixture of THF (1 mL) and water (0.5 mL) was added PPTS (30 mg) and the solution was aged for 18 h at room temperature. The reaction was then diluted with ethyl acetate, washed with sat. NaHCO₃ and water. The crude product obtained was purified on silica-gel by PTLC using ETOAc-hexane (1/2) as the eluent. The title product thus obtained was characterized by ¹H NMR and MS [m/z: 696.3 (M⁺+1)].

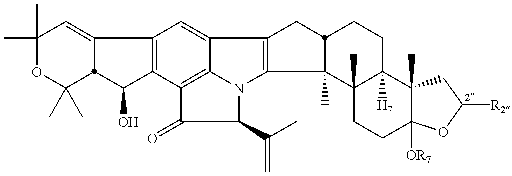

| Entry | R₇ | R_{2″} | Mass Spec |
|---|---|---|---|
| 36a | Me | CH═CHC(O)Me | 694.4(M⁺ + 1) |
| 36b | Me | CH═CHCHO | 680.2(M⁺ + 1) |
| 36c | Me | CH═CHCO₂Me | 710.3(M⁺ + 1) |
| 36d | Me | CH═CHCO₂CH₂CH═CH₂ | 736.5(M⁺ + 1) |
| 36e | Me | 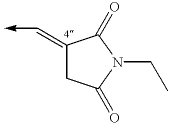 | 763.4(M⁺ + 1) |
| 36f | Me | 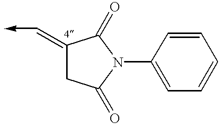 | 797.6(M⁺ + 1) |
| 36g | H | CH═CHCHO | 666.5(M⁺ + 1) |
| 36h | H | CH═CHCO₂Me | 696.3(M⁺ + 1) |
| 36i | H | CH═CHCO₂CH₂CH═CH₂ | 722.3(M⁺ + 1) |
| 36j | H | 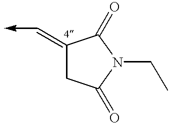 | 731.4(M⁺-H₂O + 1) |
| 36k | H | 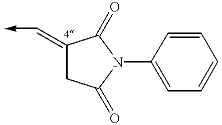 | 783.3(M⁺ + 1) |

### EXAMPLE 37

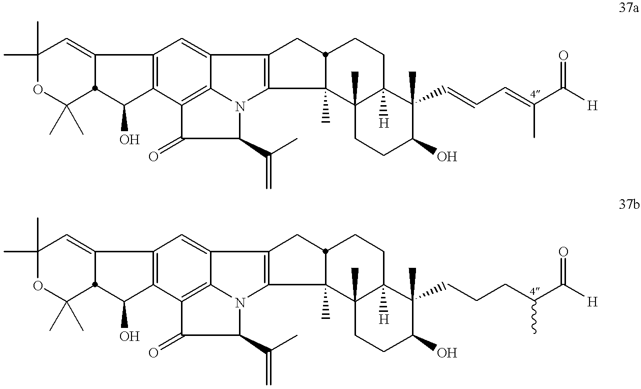

Following the general procedure of Example 29 using ethane thiol instead of cyclopropyl amine, nodulisporic acid A (compound A) was converted into the corresponding ethyl thioester. To the 5″ thioester (650 mg) thus obtained at RT was added acetone (50 mL) followed by Et3SiH (0.75 mL) and Lindlar catalyst (1.3 g). After 20 min at RT, the solids were filtered through Celite and the volatiles were removed under reduced pressure. Pure 5″ -aldehyde product 37a (420 mg) and pure 1″, 2″, 3″, 4″-tetrahydro-5″-aldehdye product 37b (70 mg) were obtained following PTLC chromatography on silica gel (5×1000 μm plates). The aldehydes thus prepared were characterized by proton NMR.

### EXAMPLE 38

Following the general olefination procedure described in Example 1 and using the aldehyde 37a, the following compounds were prepared and characterized by proton NMR:

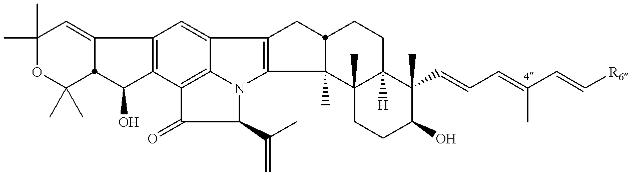

| Entry | R_{6″} Group |
|---|---|
| 38a | Ph(4-Br) (E only) |
| 38b | CO₂Et (E & Z) |
| 38c | Me (2′-epi) (E & Z) |
| 38d | Me (E & Z) |

### EXAMPLE 39

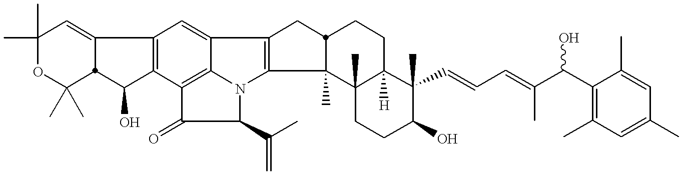

Following the general description for the preparation of Intermediate II, the C7 and C24 hydroxyl groups of compound 37a were protected as trimethylsilyl ethers. This intermediate ether was treated with 2,4,6-trimethylphenyl Grignard as described in Example 27 (step a) to form a separable mixture of 5″ -alcohol isomers. The trimethylsilyl groups were removed as described previously and the pure products (mobile isomer A; polar isomer B) obtained following PTLC were characterized by proton NMR.

### EXAMPLE 40

Following the general procedure of Example 39 and using the appropriate nucleophile, the following compounds were prepared and were characterized by proton NMR:

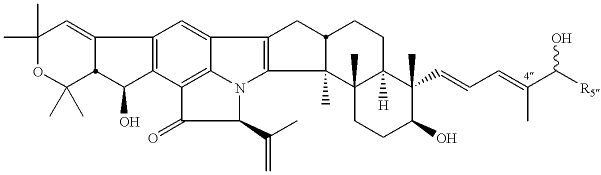

| Entry | R_{5″} Group | 5″ Isomer |
|---|---|---|
| 40a | tBu | B |
| 40b | tBu | A |
| 40c | Et | A & B |
| 40d | C≡CH | A & B |
| 40e | CH₂C(O)tBu | A |
| 40f | CH₂C(O)tBu | B |
| 40g | CH₂C(O)Ph(4-Br) | A |
| 40h | CH₂C(O)Ph(4-Br) | B |
| 40i | CH₂C(O)[2-(5-Me-furyl)] | A |
| 40j | CH₂C(O)[2-(5-Me-furyl)] | B |
| 40k | CH₂C(O)[3-(2,5-Me-thienyl)] | A |
| 401 | CH₂C(O)[3-(2,5-Me-thienyl)] | B |

### EXAMPLE 41

Following the general procedure of Example 27 (steps a and b) using the appropriate nucleophile, the following compounds were obtained and deprotected as described previously. The compounds thus obtained were characterized by proton NMR.

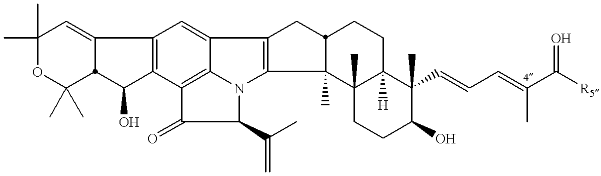

| Entry | R_{5″} Group |
|---|---|
| 41a | CH₂CO₂Ph(4-NO₂) |
| 41b | CH₂CH═CH₂(1′-CH₂CH═CH₂) |
| 41c | Et |
| 41d | tBu |
| 41e | Ph(4-tBu) |

### EXAMPLE 42

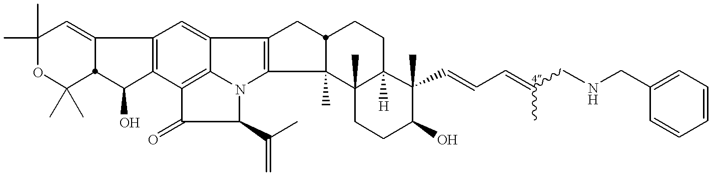

To the 5″-aldehyde 37a was placed in methanol at 0 C to which was added glacial acetic acid and benzyl amine. To the cooled solution was added NaBH3CN. The solution was aged for 3 h at 0 C and then warmed to RT. The solution was then poured into saturated brine, extracted with EtOAc and dried (Na₂SO₄). Pure product, as a mixture of olefin isomers at C4″ were obtained following PTLC and the pure product thus obtained was characterized by proton NMR.

### EXAMPLE 43

Following the general procedure of Example 42 and using an appropriate amine, the following compounds were prepared and were characterized by proton NMR:

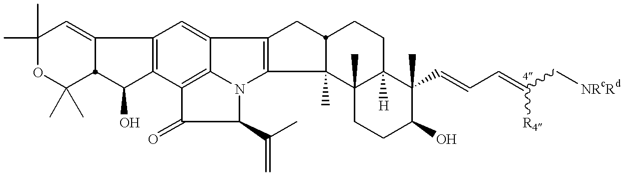

| Entry | NR^{c}R^{d}*** | R_{4″} Group |
|---|---|---|
| 43a | NHCH₂Ph | Me |
| 43b | NHCH₂Ph(4-OMe) | Me |
| 43c | NHCH₂Ph(4-OMe)(Z isomer) | Me |
| 43d | NH-tBu(E & Z isomers) | Me |
| 43e | NH-tBu | Me |
| 43f | NH-tBu(Z isomer) | Me |
| 43g | NH₂ | Me |
| 43h | N-4-morpholinyl | Me |
| 43i | N-4-morpholinyl(Z isomer) | Me |
| 43j | NHCH₂Ph(4-OCF₃) | Me |
| 43k | NHCH₂Ph(4-OCF₃)(Z isomer) | Me |
| 431 | N(Me)Et | Me |
| 43m | NHCH₂CF₂CF₃ | Me |
| 43n | N(CH₂CF₃)₂ | Me |
| 43o | N(AC)CH₂CF₂CF₃ | Me |
| 43p | N(Ac)CH₂Ph(4-OMe) | Me |
| 43q | NCH₂CH₂CH₂(N-4-morpholinyl) | Me |
| 43r | NCH₂CH₂CH₂(N-4-morpholinyl)(Z isomer) | Me |
| 43s | NMe₂(2′-nat and 2′-epi) | H |

***E isomer unless indicated

### EXAMPLE 44

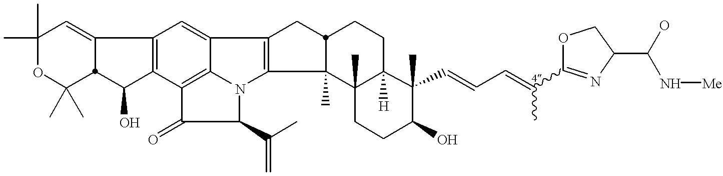

Nodulisporic acid A (compound A) was converted to the corresponding 5″-amide using H2NCH(CH₂OH)C(O)NHMe and protected as its 7,24-bis-OSiMe3 ethers as previously described. To the 7,24-bis-OSiMe3-protected-5″-amide (15 mg) thus prepared in dioxane (1.5 mL) at RT was added Burgess reagent (19 mg) and the solution was aged for 4 h. Additional Burgess reagent (19 mg) was added and the solution was aged for 12 h. Additional Burgess reagent (19 mg) was added and the solution was aged for 24 h (this was repeated twice). The volatiles were removed under reduced pressure and the pure 7,24-bis-OSiMe3-protected-4″-oxazolines were obtained following PTLC purification on silica gel (1×500 μm plate) using 2/8 acetone/hexanes as eluant. Cis (2 mg) and trans (2 mg) isomers at 4″ were isolated from this reaction. Deprotection of the trans product was accomplished as described to yield pure product (1 mg) which was characterized by ¹H NMR.

### EXAMPLE 45

Using the general strategy illustrated in Example 44, the following compounds were prepared and deprotected as previously described. The products thus obtained were characterized by proton NMR and mass spectrometry.

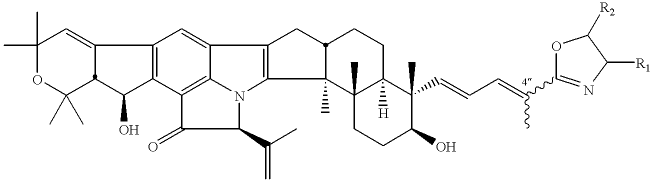

| Entry | R₁ Group | R₂ Group | Mass Spec |
|---|---|---|---|
| 45a | H | H | 705.0 (M⁺ + 1) |
| 45b | (R)-iPr | H | 747.0 (M⁺ + 1) |
| 45c | (S)-iPr | H | 747.0 (M⁺ + 1) |
| 45d | H | (S)-Me | 719.0 (M⁺ + 1) |
| 45e | H | (R)-M | 719.0 (M⁺ + 1) |
| 45f | spiro-cyclopentyl | H | 759.0 (M⁺ + 1) |

### EXAMPLE 46

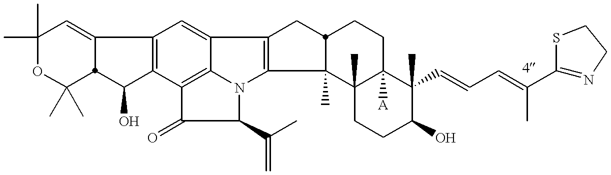

Nodulisporic acid A (Compound A) was converted to the corresponding 5″-N,N-diethyl amide as described and protected as described for Intermediate III as its 7,24-bis-OSiEt3 ether. To the bis-protected 5″-N,N-diethyl amide (40 mg) thus obtained was added pyridine (35 μL) in CH₂Cl₂ (1 mL) and the solution was cooled to −50° C. and Tf₂O (10 μL) was then added. The solution was warmed slowly to 0° C. and aged for 12 h. The solution was re-cooled to −30° C. and HSCH₂CH₂NH₂ (10 mg) was added followed by pyridine (35 μL). The solution was allowed to warm to RT. Pure 7,24-bis-OSiEt₃-protected-4″-thiazoline (16 mg) was obtained following PTLC purification on silica gel (1×1500 μm plate) using 2/8 acetone/hexanes as eluant. The 7,24-bis-OSiEt₃-protected-4″-thiazoline was deprotected as described previously to yield the desired 4″-thiazoline which was characterized by ¹H NMR.

### EXAMPLE 47

Using the general strategy illustrated in Example 46, the following compounds were prepared. The compounds thus obtained were characterized by mass spectrometry and/or proton NMR.

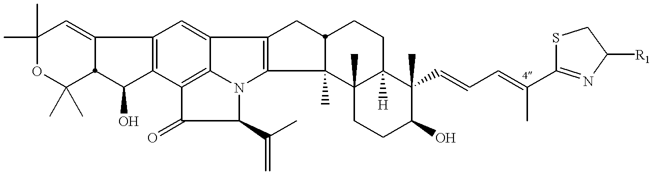

| Entry | R₁ Group*** | Mass Spec |
|---|---|---|
| 47a | (S)—C(O)NH-Me | |
| 47b | (S)—C(O)NH-Et | 776.0 (M⁺ 1) |
| 47c | (S)—C(O)NMe₂(Z isomer) | 776.0 (M⁺ + 1) |
| 47d | (S)—C(O)NMe₂ | |
| 47e | (S)—C(O)NH-tBu(Z isomer) | |
| 47f | (S)—C(O)NH-tBu | 804.0 (M⁺ + 1) |
| 47g | (S)—C(O)NH-tBu | 721.0 (M⁺ + 1) |
| 47h | CO₂Me | 779.0 (M⁺ + 1) |

***(E isomer at 4″ unless indicated)

### EXAMPLE 48

The following 4″-heterocyclic substituted derivatives were prepared and were characterized by mass spectrometry and/or proton NMR.

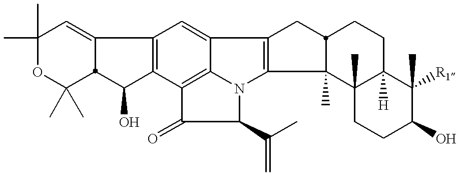

| Entry | R_{1″} Group | Mass Spec |
|---|---|---|
| 48a | 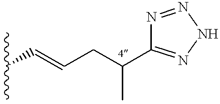 | |
| 48b | 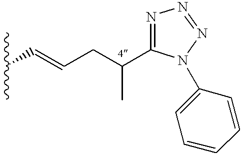 | |
| 48c | 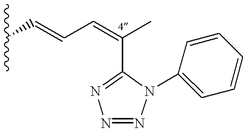 | |
| 48d | 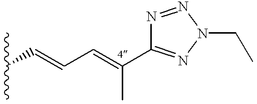 | |
| 48e | 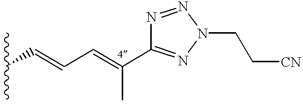 | |
| 48f | 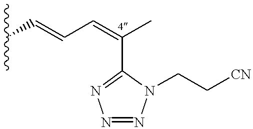 | |
| 48g | 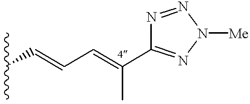 | 718.2 (M⁺ + 1) |
| 48h | 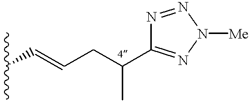 | 719.0 (M⁺ + 1) |
| 48i | 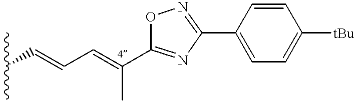 | |
| 48j | 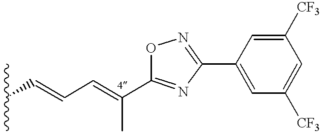 | |

### EXAMPLE 49

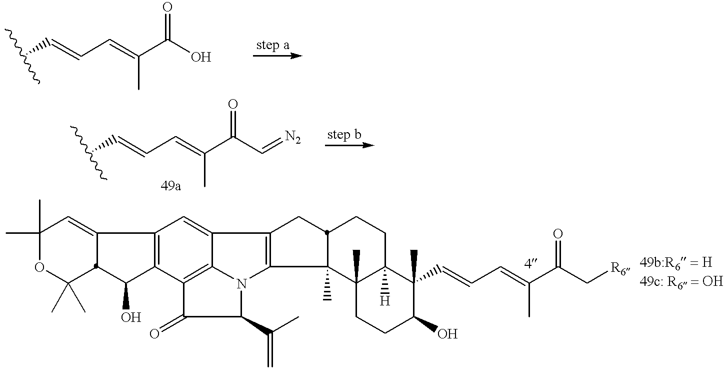

Step a:

To a solution of nodulisporic acid A (compound A, 400 mg) in THF (8 mL) maintained between 0° C. and −5° C. was added (iPr)₂NEt (0.63 mL) followed by MeSO₂Cl (0.23 mL). After 30 min at this temperature, diazomethane (excess) in diethyl ether was added and after 15 min, the solution was stirred at RT overnight. The volatiles were removed under reduced pressure and the residue was purified by radial preparative thin layer chromatography using hexanes:EtOAc (1/1) as eluant to yield pure product 49a (63 mg, 63%). The product thus obtained was characterized by proton NMR.

Step b:

The diazoketone product of step a (6.6 mg) was placed in glacial acetic acid (0.7 mL) at 0° C. to which was added LiCl (2.1 mg). After 1 h, the solution was diluted with water, extracted with ethyl acetate. The combined organic layers were washed with brine and dried (Na₂SO₄), filtered and concentrated under reduced pressure. Pure methyl ketone product 49b and alpha-hydroxy ketone product 49c were isolated following PTLC purification. The products thus obtained were characterized by proton NMR and mass spectrometry (m/z: 678.8 M⁺+1) for 49b; m/z: 694.5 M⁺+1) for 49c).

### EXAMPLE 50

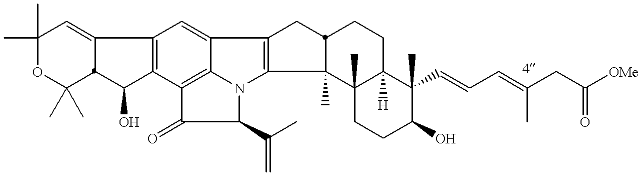

To the 4″-alpha diazoketone product of Example 49, step a (8 mg) in anhydrous methanol (0.5 mL) at RT was added triethylamine (0.04 m L) followed by PhCO₂Ag (2 mg) and the solution was aged for 1 h. The solution was filtered through a pad of Celite, concentrated to dryness under reduced pressure and the residue was washed with water, then 10% aqueous citric acid solution and then water again and then dried (Na₂SO₄). Pure product was obtained following PTLC on silica gel (1×500 m plate). The product (3 mg) thus obtained was characterized by proton NMR and mass spectrometry (m/z: 708.5 (M⁺+1).

### EXAMPLE 51

Following the general procedure of Example 50 but substituting n-propanol or allyl alcohol for methanol yielded the following ester derivatives. These compounds were characterized by proton NMR and mass spectrometry.

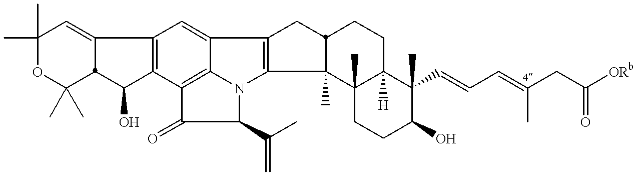

| Entry | R^{b} Group | Mass Spec |
|---|---|---|
| 51a | nPr | 736.4 (M⁺ + 1) |
| 51b | CH₂CH═CH₂ | 734.6 (M⁺ + 1) |

### EXAMPLE 52

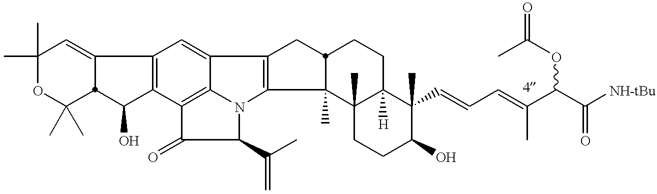

To 12.5 mg of the 5″-aldehyde 37a in methanol (0.1 mL) at RT was added water (0.01 mL), glacial acetic acid (0.02 mL) and tert-butyl isonitrile (0.02 mL). The solution was aged for 24 h at RT and then the volatiles were removed under reduced pressure. Pure product (1 mg), as a mixtures of 5″-stereoisomers was obtained following PTLC purification using EtOAc:hexanes (1/1) as eluant. The product thus obtained was characterized by proton NMR.

### EXAMPLE 53

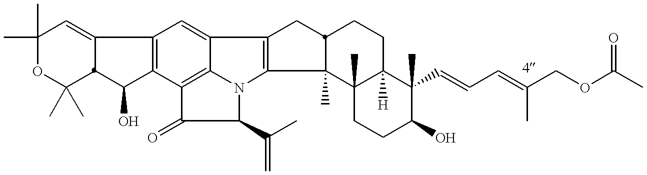

To the 5″-aldehyde 37a (9 mg) was added glacial acetic acid (0.2 mL) followed by NaBH₃CN (20 mg). After 30 min, saturated aqueous NaHCO3 was added followed by EtOAc extraction. Pure 5″-hydroxylmethyl product (5 mg) was obtained following PTLC purification using hexanes:acetone (6/4) as eluant. The product thus obtained was characterized by proton NMR. To the 5″-hydroxymethyl derivative (3 mg) thus obtained at RT was added acetic anhydride (0.1 mL) followed by (iPr)₂NEt (0.1 ImL). After 30 min, the volatiles were removed under reduced pressure and pure 5″-acetoxymethyl derivative was obtained following PTLC chromatography on silica gel using hexanes:EtOAc (2:1) as eluant. The product thus obtained was characterized by proton NMR.

### EXAMPLE 54

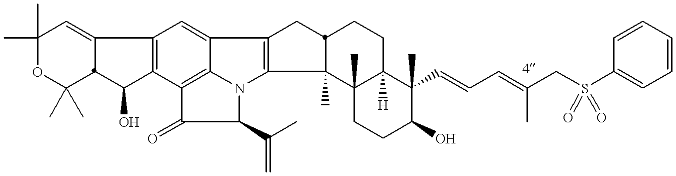

To the 5″-acetoxymethyl product (12 mg) of Example 53 at RT in a one dram glass vial was added methanol (1 mL) followed by PhSO₂Na (20 mg) and Pd(PPh₃)₄ (10 mg) and an argon atmosphere was established. The vial was tightly sealed and then heated to 65° C. for 2 h. The solution was cooled to RT and the volatiles were removed under reduced pressure. Pure 5″-sulfone product (4.6 mg) was obtained following PTLC purification on silica gel using hexanes:EtOAc (2/1) as eluant. The product thus obtained was characterized by proton NMR.

### EXAMPLE 55

Following the general procedure of Example 54 and using an appropriate sulfenic acid salt, the following compounds were prepared and were characterized by proton NMR.

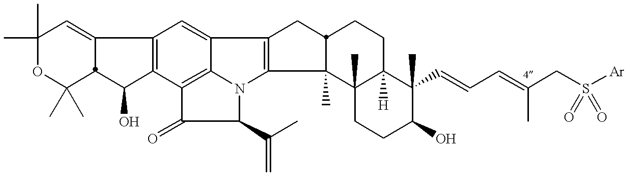

| Entry | Ar Group |
|---|---|
| 55a | Ph(4-Br) |
| 55b | Ph(3,4-Cl) |
| 55c | Ph(3-OAc) |

### EXAMPLE 56

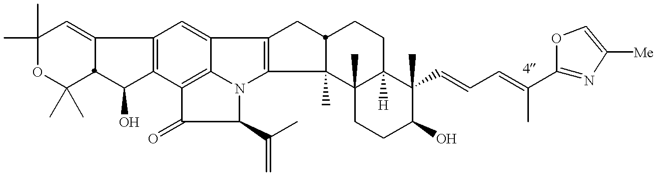

Step A.

To the 4″-oxazoline product of Example 45d (100 mg) at RT in pyridine/DMF (5 mL, 1/1) add Et3SiOSO2CF3 (460 mg) and stir for 30 min. Dilute the solution with EtOAc, wash with saturated CuSO4(aq), water, brine and dry the organic layer (Na2SO4). Filter and concentrate the residue under reduced pressure. Pure 7,24-bis-OSiEt3 protected 52d may be obtained following flash chromatography on silica gel.

Step B.

Place the 7,24-bis-OSiEt3 protected 52d (17 mg) thus prepared in methylene chloride (0.3 mL) at 0° C. add BrCCl3 (3 μL) followed by DBU (3.6 μL) and age for 12 h. Pour the solution into saturated NaHCO3(aq), extract with CH2Cl2 and dry (Na2SO4). Filter the solution and then concentrate under reduced pressure. Pure 7,24-bis-OSiEt3 protected oxazole-containing product may be obtained following PTLC on silica gel. The 7,724-bis-OSiEt3 protecting groups may be removed as previously described to yield the desired deprotected title oxazolyl product.

### EXAMPLE 57

Using the general strategy illustrated in Example 56 and the appropriate oxazoline precursors prepared as described in Example 44, the following oxazolyl compounds may be prepared. In addition, the allyl groups of 57h, 57i or 57j may be removed to liberate the corresponding carboxylic acids and the resultant carboxylic acids may be converted into the corresponding amide derivatives as previously described. The products thus obtained may be characterized by proton NMR and mass spectrometry.

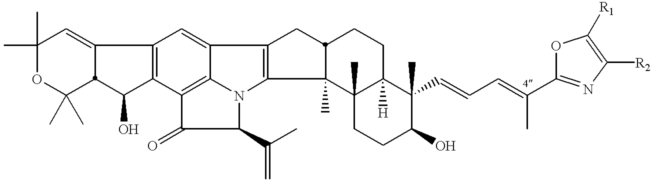

| Entry | R₁ Group | R₂ Group |
|---|---|---|
| 57a | H | H |
| 57b | iPr | H |
| 57c | H | iPr |
| 57d | Me | H |
| 57e | H | Me |
| 57f | H | C(O)NH-Me |
| 57g | H | Ph |
| 57h | CO₂CH₂CH═CH₂ | H |
| 57i | H | CO₂CH₂CH═CH₂ |
| 57j | Me | CO₂CH₂CH═CH₂ |
| 57k | H | CO₂H |
| 57l | H | C(O)NH₂ |
| 57m | Me | C(O)NH₂ |
| 57n | H | C(O)NH-Me |
| 57o | C(O)NH-Et | H |
| 57p | H | C(O)NH-iPr |
| 57q | H | C(O)NH-cPr |
| 57r | H | C(O)NH-tBu |
| 57s | H | C(O)NHCH₂CH₂F |
| 57t | Me | C(O)NHCH₂CH₂F |
| 57u | H | C(O)NHCH₂CF₃ |
| 57v | H | C(O)NHCH₂CN |
| 57w | H | C(O)NHCH₂C(Me)═CH₂ |
| 57x | H | C(O)NHC(Me)₂C≡CH |
| 57y | H | C(O)NHC(Me)₂C(O)NMe₂ |
| 57z | H | C(O)NHCH₂Ph(4-OMe) |
| 57aa | H | C(O)NMe₂ |
| 57bb | H | C(O)NEt₂ |
| 57cc | H | C(O)N(Me)Et |
| 57dd | H | C(O)N(Me)iPr |
| 57ee | H | C(O)N(Et)iPr |
| 57ff | H | C(O)(N-1-piperidinyl) |
| 57gg | H | C(O)(N-1-pyrrolidinyl) |
| 57hh | H | C(O)(N-4-morpholinyl) |

### EXAMPLE 58

Using the general strategy illustrated in Example 56 and the appropriate thiazoline precursors (in lieu of the oxazoline precursors) prepared as described in Example 46, the following thiazolyl compounds may be prepared. In addition, the allyl groups of 58h, 58i or 58j may be removed to liberate the corresponding carboxylic acids and the resultant carboxylic acids may be converted into the corresponding amide derivatives as previously described. The products thus obtained may be characterized by proton NMR and mass spectrometry.

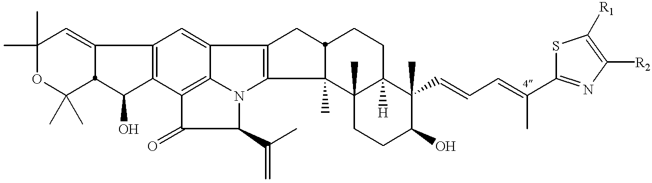

| Entry | R₁ Group | R₂ Group |
|---|---|---|
| 58a | H | H |
| 58b | iPr | H |
| 58c | H | iPr |
| 58d | Me | H |
| 58e | H | Me |
| 58f | H | C(O)NH-Me |
| 58g | H | Ph |
| 58h | CO₂CH₂CH═CH₂ | H |
| 58i | H | CO₂CH₂CH═CH₂ |
| 58j | Me | CO₂CH₂CH═CH₂ |
| 58k | H | CO₂H |
| 58l | H | C(O)NH₂ |
| 58m | Me | C(O)NH₂ |
| 58n | H | C(O)NH-Me |
| 58o | C(O)NH-Et | H |
| 58p | H | C(O)NH-iPr |
| 58q | H | C(O)NH-cPr |
| 58r | H | C(O)NH-tBu |
| 58s | H | C(O)NHCH₂CH₂F |
| 58t | Me | C(O)NHCH₂CH₂F |
| 58u | H | C(O)NHCH₂CF₃ |
| 58v | H | C(O)NHCH₂CN |
| 58w | H | C(O)NHCH₂C(Me)═CH₂ |
| 58x | H | C(O)NHC(Me)₂C≡CH |
| 58y | H | C(O)NHC(Me)₂C(O)NMe₂ |
| 58z | H | C(O)NHCH₂Ph(4-OMe) |
| 58aa | H | C(O)NMe₂ |
| 58bb | H | C(O)NEt₂ |
| 58cc | H | C(O)N(Me)Et |
| 58dd | H | C(O)N(Me)iPr |
| 58ee | H | C(O)N(Et)iPr |
| 58ff | H | C(O)(N-1-piperidinyl) |
| 58gg | H | C(O)(N-1-pyrrolidinyl) |
| 58hh | H | C(O)(N-4-morpholinyl) |

What is claimed is:

1. 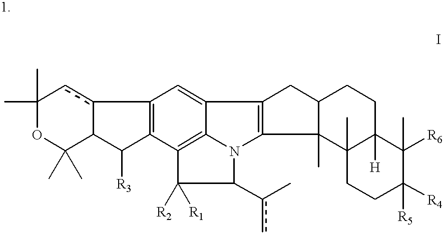
wherein
R₁ is
(1) hydrogen,
(2) optionally substituted C₁-C₁₀ alkyl,
(3) optionally substituted C₂-C₁₀ alkenyl,
(4) optionally substituted C₂-C₁₀ alkynyl,
(5) optionally substituted C₃-C₈ cycloalkyl,
(6) optionally substituted C₅-C₈ cycloalkenyl where the substitutents on the alkyl, alkenyl, alkynyl, cycloalkyl and cycloalkenyl are 1 to 3 groups independently selected from
(i) C₁-C₅ alkyl,
(ii) X—C₁-C₁₀ alkyl,
(iii) C₃-C₈ cycloalkyl,
(iv) hydroxy,
(v) halogen,
(vi) cyano,
(vii) carboxy,
(viii) NY¹Y²,
(ix) C₁-Clo alkanoylamino, and
(x) aroyl amino wherein said aroyl is optionally substituted with 1 to 3 groups independently selected from R^{f}
(7) aryl C₀-C₅ alkyl wherein said aryl is optionally substituted with 1 to 3 groups independently selected from R^{f},
(8) C₁-C₅ perfluoroalkyl
(9) a 5- or 6-membered heterocycle optionally substituted by 1 to 3 groups independently selected from hydroxy, oxo, C₁-C₁₀ alkyl and halogen;
R₂, R₃, and R₄ are independently OR^{a}, OCO₂R^{b}, OC(O)NR^{c}R^{d}; or
R¹+R² represent ═O, ═NORa, ═N—NR^{c}R^{d}, ═CCO₂R^{a}, ═CC(O)NR^{c}R^{d}, ═CCN ═CC(O)R^{a}, or ═CR^{a}R^{a};
R₅ is hydrogen, OR^{a} or R₄+R₅ represent ═O, ═NOR^{a}, ═N—NR^{c}R^{d} or ═CR^{a}R^{a};
R₆ is
(1) the fragment R₂₀ 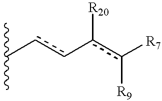
(2) the fragment 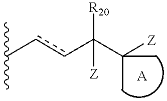
where A is a 5- or 6-membered heterocycle optionally substituted with 1 to 4 groups independently selected from C₁-C₅ alkyl, C₂-C₅ alkenyl, C₁-C₅ perfluoroalkyl, aryl, OR^{a}, NR^{c}R^{d}, oxo, thiono, C(O)Ra, C(O)NR^{c}R^{d}, cyano, CO₂R^{b} and halogen, and where a ring nitrogen is present, it is substituted with a group selected from R^{c}; the two Z groups are each OH or together form a bond across the two carbon atoms to which they are attached;
(3) the fragment 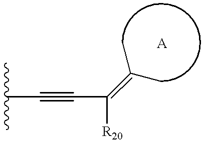
wherein A is as defined above; or
R⁶, R⁴ and the atoms to which they are attached together form the fragment 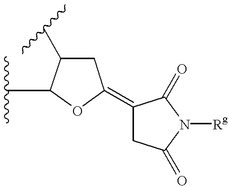
R⁷ is
(1) hydrogen,
(2) optionally substituted C₁-C₁₀ alkyl,
(3) optionally substituted C₂-C₁₀ alkenyl,
(4) optionally substituted C₂-C₁₀ alkynyl,
(5) optionally substituted aryl,
(6) optionally substituted C₃-C₈ cycloalkyl,
(7) optionally substituted C₅-C₈ cycloalkenyl,
(8) halogen,
(9) CN,
(10) C(O)R^{a},
(11) CH═NOR^{a},
(12) CO₂R^{b},
(13) C(O)NR^{c}R^{d},
(14) C(O)N(OR^{b})R^{c},
(15) C(O)NR^{c}NR^{c}R^{d},
(16) C(O)NR^{c}SO₂R^{b},
(17) NR^{c}R^{d},
(18) NR^{c}C(O)R^{a},
(19) NR^{c}C(O)OR^{b},
(20) NR^{c}C(O)NR^{c}R^{d},
(21) NR^{c}C(O)SR^{b},
(22) NR^{c}C(O)P(O)(R^{a})₂,
(23) NR^{c}S(O)₂R^{a},
(24) N═C═O,
(25) XR^{a},
(26) OC(O)R^{a},
(27) OSO₂R^{a},
(28) P(O)(OR^{a})2,
(29) 4- to 8-membered heterocycle optionally substituted by 1 to 4 groups independently selected from C₁-C₅ alkyl, C₂-C₅ alkenyl, C₁-C₅ perfluoroalkyl, NRCRd, oxo, thiono, C(O)NRCRd, cyano, aryl, C(O)Ra, CO₂Rb and halogen,
where the substituents on the optionally substituted alkyl, alkenyl, alkynyl, aryl, cycloalkyl and cycloalkenyl are from 1 to 10 groups independently selected from
(a) halogen,
(b) C₃-C₇ cycloalkyl,
(c) C₁-C₇ alkyl optionally substituted with from 1 to 3 groups independently selected from OR^{a}, oxo, NR^{c}R^{d}, N₃, NR^{c}C(O)R^{a}, NR^{c}SO₂R^{a}, O₂CNR^{c}R^{d}, NR^{c}C(O)NR^{c}R^{d}, CO₂R^{b}, C(O)NR^{c}R^{d}, or a 3- to 8-membered heterocycle optionally substituted with oxo or C(O)R^{a},
(d) C₁-C₇ alkenyl optionally substituted with from 1 to 3 groups independently selected from OR^{a}, oxo, NR^{c}R^{d}, N₃, NR^{c}C(O)R^{a}, NR^{c}SO₂R^{a}, O₂CNR^{c}R^{d}, NR^{c}C(O)R^{c}R^{d}, CO₂R^{b}, C(O)NR^{c}R^{d}, or a 3- to 8-membered heterocycle optionally substituted with oxo or C(O)R^{a},
(e) C₁-C₅ perfluoroalkyl,
(f) aryl optionally substituted with 1 to 3 groups selected from R^{f},
(g) CN,
(h) C(O)R^{a},
(i) CO₂R^{b},
(j) C(O)NR^{c}R^{d},
(k) C(O)N(OR^{b})R^{c},
(l) C(O)NR^{c}NR^{c}R^{d},
(m)C(O)NR^{c}SO₂R^{b},
(n) N═C═O,
(o) N═N═N,
(p) NR^{c}C(O)NR^{c}R^{d},
(q) NR^{c}C(O)P(O)R^{a},
(r) NR^{c}R^{d},
(s) NR^{c}CO₂R^{b},
(t) NR^{c}SO₂R^{a},
(u) NR^{c}C(O)SR^{b},
(v) NR^{c}C(O)R^{a},
(w) ═NOR^{a},
(x) ═NNR^{c}R^{d},
(y) ═NNR^{c}SO₂R^{a}
(z) XR^{a},
(aa) oxo,
(bb) OCO₂R^{b},
(cc) OC(O)NR^{c}R^{d},
(dd) OSO₂R^{a},
(ee) P(O)(OR^{a})₂,
(ff) a 4- to 8-membered heterocycle optionally substituted by 1 to 4 groups independently selected from C₁-C₅ alkyl, C₂-C₅ alkenyl, C₁-C₅ perfluoroalkyl, NR^{c}R^{d}, oxo, thiono, XR^{a}, C(O)NR^{c}R^{d}, cyano, (C₀-C₆-alkyl)aryl, CO₂R^{b} and halogen;
R₉ is a group selected from R₇, with the proviso that R₉ is not methyl when R₇ is CN, C(O)OR^{b}, C(O)N(OR^{b})R^{c}, C(O)NR^{c}R^{d}, NHC(O)OR^{b}, NHC(O)NR^{c}R^{d}, CH₂OR^{a}, CH₂OCO₂R^{b}, CH₂OC(O)NR^{c}R^{d}, C(O)NR^{c}NR^{c}R^{d}, or C(O)NR^{c}SO₂R^{b};
R₂₀ is a group selected from R₇;
___ represents a single or a double bond;
R^{a} is
(1) hydrogen,
(2) optionally substituted C₁-C₁₀ alkyl,
(3) optionally substituted C₃-C₁₀ alkenyl,
(4) optionally substituted C₃-C₁₀ alkynyl,
(5) optionally substituted C₁-C₁₀ alkanoyl,
(6) optionally substituted C₃-C₁₀ alkenoyl,
(7) optionally substituted C₃-C₁₀ alkynoyl,
(8) optionally substituted aroyl,
(9) optionally substituted aryl,
(10) optionally substituted C₃-C₇ cycloalkanoyl,
(11) optionally substituted C₅-C₇ cycloalkenoyl,
(12) optionally substituted C₁-C₁₀ alkylsulfonyl,
(13) optionally substituted C₃-C₈ cycloalkyl,
(14) optionally substituted (C₁-C₆ alkyl)aryl,
(15) optionally substituted C₅-C₈ cycloalkenyl,
(16) C₁-C₅ perfluoroalkyl,
(17) arylsulfonyl optionally substituted with 1 to 3 groups independently selected from C₁-C₅ alkyl, C₁-C₅ perfluoroalkyl, nitro, halogen and cyano,
(18) a 4- to 8-membered heterocycle optionally substituted with 1 to 4 groups independently selected from C₁-C₅ alkyl, C₂-C₅ alkenyl, C₁-C₅ perfluoroalkyl, NR^{g}R^{h}, oxo, thiono, OH, C₁-C₅ alkoxy, C₁-C₅ alkanoyl, C(O)NR^{g}R^{h}, cyano, CO2H, CO₂-C₁-C₅ alkyl and halogen;
where the substituents on the optionally substituted alkyl, alkenyl, alkynyl, alkanoyl, alkenoyl, alkynoyl, aroyl, aryl, cycloalkanoyl, cycloalkenoyl, alkylsulfonyl, cycloalkyl and cycloalkenyl are from 1 to 10 groups independently selected from OH, C₁-C₆ alkoxy, C₁-C₆ alkyl, C₃-C₇ cycloalkyl, aryl C₁-C₃ alkoxy, NR^{g}R^{h}, CO₂H, CO₂C₁-C₆ alkyl, C(O)NR^{g}R^{h}, NR^{g}C(O)C₁-C₆ alkyl and halogen,
R^{b} is
(1) H,
(2) optionally substituted aryl,
(3) optionally substituted C₁-C₁₀ alkyl,
(4) optionally substituted C₃-C₁₀ alkenyl,
(5) optionally substituted C₃-C₁₀ alkynyl,
(6) optionally substituted C₃-C₁₅ cycloalkyl,
(7) optionally substituted C₀-C₆ alkyl S(O)₂Rⁱ,
(8) optionally substituted C₂-C₆ alkanoyl,
(9) optionally substituted C₅-C₁₀ cycloalkenyl, or
(10) optionally substituted 4- to 8-membered heterocycle;
where the substituents on the optionally substituted aryl, alkyl, alkenyl, cycloalkyl, cycloalkenyl, heterocycle, or alkynyl are from 1 to 10 groups independently selected from
(a) C₁-C₆ alkyl optionally substituted with aryl, hydroxy or amino,
(b) C₁-C₅ perfluoroalkyl;
(c) C₃-C₇ cycloalkyl optionally substituted with 1 to 4 groups independently selected from R^{e},
(d) C₅-C₇ cycloalkenyl,
(e) halogen,
(f) cyano,
(g) OH,
(h) XC₁-C₆ alkyl optionally substituted with amino, hydroxy or aryl optionally substituted with 1,2-methylenedioxy or 1 to 5 groups independently selected from R^{e},
(i) OC(O)C₁-C₅ alkyl,
(j) aryl C₁-C₆ perfluoroalkoxy,
(k) oxo,
(l) SO₂NR^{g}R^{h},
(m)C(O)Rⁱ,
(n) CO₂Rⁱ,
(o) C(O)NR^{g}R^{h},
(p) NR^{g}R^{h},
(q) N(R^{g})CO₂Rⁱ,
(r) N(R^{c})c(S)ORⁱ,
(s) 4- to 8-membered heterocycle optionally substituted with 1 to 5 groups independently selected from Re, and
(t) aryl optionally substituted with 1,2-methylenedioxy or 1 to groups independently selected from R^{e},
R^{c} and R^{d} are independently selected from R^{b}; or
R^{e} and R^{d} together with the N to which they are attached form a 3- to 10-membered ring containing 0 to 2 additional heteroatoms selected from O, S(O)ₘ, and NR^{g}, optionally substituted with 1 to 3 groups independently selected from R^{g}, hydroxy, thiono and oxo;
R^{e} is
(1) halogen,
(2) C₁-C₇ alkyl,
(3) C₁-C₃ perfluoroalkyl,
(4) cyano,
(5) nitro,
(6) RⁱX(CH₂)_{v—},
(7) RⁱCO2(CH₂)_{v—},
(8) RⁱOCO(CH₂)ᵥ,
(9) aryl optionally substituted with from 1 to 3 of halogen, C₁-C₆ alkyl, C₁-C₆ alkoxy, or hydroxy,
(10) So₂NR^{g}R
(11) amino, or
(12) oxo;
R^{f} is
(1) C₁-C₄ alkyl,
(2) C₂-C₄ alkenyl,
(3) C₂-C₄ alkynyl,
(4) C₁-C₃-perfluoroalkyl,
(5) NY¹Y²,
(6) NHC(O)C₁-C₅ alkyl,
(7) OH,
(8) X—C₁-C₄ alkyl, or
(9) halogen,
(10) R^{g} and R^{h} are independently
(11) hydrogen,
(12) C₁-C₆ alkyl optionally substituted with hydroxy, amino, C₁-C₅ alkanoyl or CO₂Rⁱ
(13) C₀-C₆aryl optionally substituted with halogen, 1,2-methylene-dioxy, C₁-C₇ alkoxy, C₁-C₇ alkyl or C₁-C₃ perfluoroalkyl,
(14) C(O)OC₁-C₅ alkyl optionally substituted with aryl,
(15) C(O)C₁-C₅ alkyl,
(16) C(O)NY¹Y², or
R^{g} and R^{h} together with the N to which they are attached form a 3- to 7-membered ring containing 0 to 2 additional heteroatoms selected from O, S(O)ₘ, and NRⁱ, optionally substituted with 1 to 3 groups independently selected from halogen, C₁-C₇ alkyl, C₁-C₃ perfluoroalkyl, cyano, nitro, RⁱX(CH₂)_{v—}, RⁱCO₂(CH₂)_{v—}, RⁱOCO(CH₂)ᵥ, aryl optionally substituted with from 1 to 3 of halogen, C₁-C₆ alkyl, C₁-C₆ alkoxy, or hydroxy, and oxo;
Rⁱ is
(1) hydrogen,
(2) C₁-C₃ perfluoroalkyl,
(3) C₁-C₆ alkyl,
(4) optionally substituted aryl C₀-C₆ alkyl, where the aryl substituents are from 1 to 3 groups independently selected from halogen, C₁-C₆ alkyl, C₁-C₆ alkoxy, and hydroxy;
X is O or S(O)ₘ,
Y¹ and Y² are independently hydrogen or C₁-C₅ alkyl,
m is 0 to 2;and
v is 0 to 3;or
a pharmaceutically acceptable salt thereof.

2. A compound of claim 1 having the formula Ia:

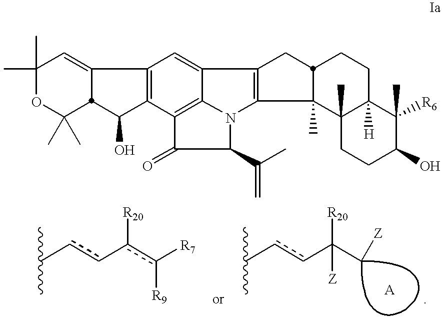

3. A compound of claim 2 wherein R₆ is the fragment 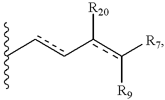
----- represents a double bond, and R₂₀ is hydrogen or C₁-C₅ alkyl.

4. A compound of claim 3 wherein R₇ is CN, CO₂R^{b} or CO₂NR^{c}R^{d}, and R^{g} is other than methyl.

3. A compound of claim 4 wherein R₉ is selected from hydrogen, halogen, cyano, OC₁-C₅ alkyl, trifluoromethyl, hydroxymethyl, CH₂C(O)NR^{c}R^{d}, NR^{c}C(O)OR^{a}, optionally substituted C₂-C₆ alkyl, optionally substituted C₂-C₆ alkenyl, and optionally substituted aryl wherein said substituents for alkyl, alkenyl and aryl are 1 to 10 groups selected from halogen, OR^{a}, OC(O)NR^{c}R^{d}, oxo, NR^{c}R^{d}, and NR^{c}C(O)NR^{c}R^{d}.

5. A compound of claim 3 wherein R₇ is selected from hydrogen, NR^{c}C(O)R^{a}, NR^{c}C(O)NR^{c}R^{d}, NR^{c}C(O)OR^{b}, C(O)R^{a}, P(O)(OR^{a})₂, optionally substituted heterocycle, optionally substituted aryl, CH═NOR^{a}, OSO₂R^{a}, NR^{c}C(O)P(O)(R^{a})₂, NR^{c}C(O)SR^{b}, substituted methyl wherein the substitutents are selected from CO₂R^{b}, C(O)R^{a}, NR^{c}R^{d}, XR^{a}, and wherein the substituents of optionally substituted heterocycle and optionally substituted aryl are as defined in claim 3.

6. A compound of claim 5 wherein R₉ is selected from hydrogen, halogen, cyano, OC₁-C₅ alkyl, NR^{c}C(O)OR^{a}, optionally substituted C₁-C₆ alkyl, optionally substituted C₂-C₆ alkenyl, and optionally substituted aryl wherein said substituents for alkyl, alkenyl and aryl are 1 to 10 groups selected from halogen, OR^{a}, OC(O)NR^{c}R^{d}, oxo, NR^{c}R^{d}, and NR^{c}C(O)NR^{c}R^{d}.

7. A compound of claim 2 wherein R₆ is the fragment 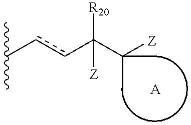
___ wherein R₂₀ is hydrogen, [symbol] is a double bond, two Z groups form a bond across the carbon atoms to which they are attached, and A is selected
from 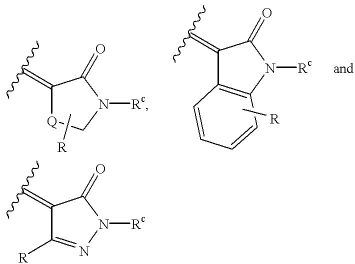
wherein Q is C, N or S, and R is H or a ring A substituent.

8. A pharmaceutical composition comprising a compound of claim 1 and a pharmaceutically acceptable carrier.

9. A composition of claim 8 further comprising an anthelmintic agent.

10. A composition of claim 9 wherein said anthelmintic agent is selected from the group consisting of: ivermectin, avermectin 5-oxime, abamectin, emamectin, eprinamectin, doramectin, doramectin monosaccharide 5-oximes, fulladectin, milbemycin, milbamycin 5-oxime, moxidectin, Interceptor™, nemadectin, imidacloprid, fipronil, lufenuron, thiabendazole, cambendazole, parbendazole, oxibendazole, mebendazole, flubendazole, fenbendazole, oxfendazole, albendazole, cyclobendazole, febantel, thiophanate, tetramisole-levamisole, butamisole, pyrantel, pamoate, oxantel and morantel.

11. A composition of claim 8 further comprising fipronil, imidacloprid, lufenuron or an ecdysone agonist.

12. A method for the treatment or prevention of a parasitic disease in a mammal which comprises administering to said mammal an antiparasitic effective amount of a compound of claim 1.

13. A method of claim 12 further comprising administering an anthelmintic agent.

14. A method of claim 12 further comprising administering fipronil, imidacloprid or lufenuron.
